(19) United States
(12) Patent Application Publication
(10) Pub. No.: US 2023/0220047 A1
(43) Pub. Date: Jul. 13, 2023

(54) PEPTIDES FOR TREATMENT OF SEPSIS AND CANCER

(71) Applicant: NORWEGIAN UNIVERSITY OF SCIENCE AND TECHNOLOGY (NTNU), Trondheim (NO)

(72) Inventors: Maria YURCHENKO, Trondheim (NO); Terje ESPEVIK, Trondheim (NO); Harald HUSEBYE, Trondheim (NO)

(21) Appl. No.: 18/008,856

(22) PCT Filed: Jun. 10, 2021
(86) PCT No.: PCT/EP2021/065718

§ 371 Date: Dec. 7, 2022

(30) Foreign Application Priority Data
Jun. 11, 2020 (GB) 2008888.6

(51) Int. Cl. C07K 14/705; A61P 35/00

(52) U.S. Cl. CPC C07K 14/70596; A61P 35/00; C07K 2319/10

(57) ABSTRACT

The present invention provides oligopeptidic compounds comprising a first oligopeptidic component derived from SLAMF1 and a second oligopeptidic component which is a cell-penetrating peptide. The oligopeptidic compounds provided have been found unexpectedly to block signalling from TLR4, TLR7, TLR8 and TLR9 in response to stimulus by their ligands, and also to display an anti-proliferative effect on cancer cells. The oligopeptidic compounds provided may be used in therapy for conditions associated with overactive immune responses, such as sepsis, and for treatment of cancer.

### FIELD

The present invention is directed to oligopeptidic compounds capable of entering target cells and binding to TRAM, and inhibiting its interaction with SLAMF1, and/or inhibiting the formation of the Myddosome complex, such that signalling from the toll-like receptors TLR4, TLR7, TLR8 and/or TLR9 is blocked, and cytokine expression is reduced. Also provided herein are oligopeptidic compounds which have an anti-proliferative and/or cytotoxic effect on cancer cells. The invention also includes uses of these compounds (including in therapy), kits, products and compositions comprising these compounds, and nucleic acid molecules encoding such compounds. The compounds may in particular be used in the treatment or prevention of hypercytokinemia-associated diseases in human subjects, such as sepsis, and in the treatment of cancer. The compounds are also useful in therapy for a variety of other diseases, including autoimmune diseases.

### BACKGROUND

Toll-like receptor 4 (TLR4) is a pattern recognition receptor (PRR) primarily expressed on the surface of immune cells. TLR4 recognises pathogen-associated ligands, in particular lipopolysaccharide (LPS) from Gram-negative bacteria, which it binds in the context of a complex with the co-receptors myeloid differentiation factor 2 (MD-2) and CD14. TLR4 can also bind lipoteichoic acid (LTA) from the cell wall of some Gram-positive bacteria. Binding of LPS (or other ligand molecules) to TLR4 activates the receptor, resulting in recruitment of the signalling adaptor MyD88-adaptor-like protein (Mal, also known as TIRAP, for toll/interleukin-1 receptor domain-containing adapter protein), which recruits Myeloid Differentiation primary response protein 88 (MyD88) (Bernard & O'Neill, IUBMB LIFE 65(9): 777-786 (2013)), which in turn recruits interleukin 1 receptor-associated kinase 4 (IRAK4). IRAK4 dimerisation following recruitment by MyD88 leads to recruitment of interleukin 1 receptor-associated kinase 1 (IRAK1). The complex formed between Mal, MyD88, IRAK1 and IRAK4 is known as the Myddosome complex. Recruitment of IRAK1 by IRAK4, which completes formation of the Myddosome complex, leads to activation of IRAK1 (possibly as a result of its dimerization, or by an allosteric mechanism as a result of IRAK4/MyD88 binding, Vollmer et al., Biochemical Journal 474(12): 2027-2038 (2017)). Active IRAK1 binds and activates the downstream effector TRAF6. TRAF6 activation ultimately leads to activation of the transcription factors NF-κB and activator protein 1 (AP1), which contributes to the activation of expression of pro-inflammatory cytokines including IL-1β and IL-6 (Wang et al., PNAS 114(51): 13507-13512 (2017)).

Indeed, the Myddosome mediates signalling from multiple TLR receptors, not just TLR4. Other TLRs which utilise the Myddosome signalling pathway include TLR2, which forms heterodimers with TLR1 and TLR6 that primarily recognise cell wall components from Gram-positive bacteria and yeast, including LTA, peptidoglycan, lipoproteins and lipoglycans, and some viral glycoproteins; TLR7 and TLR8, which recognise single-stranded RNA from RNA viruses; and TLR9, which recognises unmethylated bacterial DNA and viral DNA. In addition to its well-known role in virus recognition, TLR8 has also recently be found to play an important role in sensing of some Gram-positive bacterial species (particularly Group B Streptococci), presumably through binding of bacterial RNA. In addition to activation of expression of the cytokines mentioned above, signalling through the Myddosome from TLR7, TLR8 and TLR9 can also activate expression of the Type I interferon interferon-α (IFNα) in dendritic cells.

Binding of a ligand to TLR4 results in ligand-stimulated endocytosis, following which TLR4 is located on the surface of an endosome or phagosome. At this point the signalling adaptor TRIF-Related Adaptor Molecule (TRAM) is recruited to the activated TLR4 (Kagan, J. C. et al., Nature Immunology 9: 361-368 (2008)). TRAM is crucial for subsequent recruitment of TIR-domain-containing adaptor-inducing Interferon-β (TRIF) and other downstream molecules, which initiates a second signaling pathway from TLR4 leading to secretion of pro-inflammatory cytokines including interferon-β (IFNβ) and tumour necrosis factor (TNF) (Fitzgerald, K. A. et al., The Journal of Experimental Medicine 198: 1043-1055 (2003)). Signalling through this TRAM-mediated pathway is unique amongst the TLRs to TLR4.

The role of endogenous type I IFNs (such as IFNα and IFNβ) in host defense against bacterial infections can be beneficial or detrimental. Type I IFNs are required for host resistance to Group B Streptococci, Pneumococci and E. coli. However, an excessive Type I interferon response is associated with hypercytokinemia (also known as a cytokine storm), a condition in which uncontrolled positive feedback between proinflammatory cytokines and white blood cells leads to tissue damage and, if untreated, organ failure and death.

Despite the advances in medical science in recent years, hypercytokinemia remains notoriously difficult to treat. Hypercytokinemia-associated diseases have high mortality rates, and new and improved treatments are urgently needed. Type I interferons, particularly IFNβ, have been linked in particular to sepsis and septic shock, conditions in which an infection, most commonly a bacterial infection, leads to hypercytokinemia. Hypercytokinemia can also result from viral infections, and is a major cause of mortality from COVID-19 (resulting from infection with SARS-CoV-2). Currently, treatment for sepsis is limited to treatment of the underlying infection (where possible) in combination with supportive therapy for the body and organs. An as yet untested approach is to target the host immune system itself, to try and dampen the cytokine storm. Such an approach has, however, been suggested, for instance inhibition of Type I interferons has been postulated as a therapy for sepsis (Mahieu & Libert, Infection and Immunity 75: 22-29 (2007)), as has blockage of TLR4 signalling and resultant cytokine production (US 2010/0069297). Targeting of Type I interferons is thus well-established as a putative sepsis treatment. However, no such therapy has as yet entered the clinic, and mortality from sepsis remains as high as 35%, meaning new and improved treatments for the condition are urgently needed.

NF-κB activation (which as noted above is a downstream effect of TLR4 signalling) is also known to play a role in the development and progression of certain cancers, and in the development of cancer drug resistance. Inhibition of NF-κB activation has been shown to suppress proliferation of certain cancer cells (particularly multiple myeloma cell lines) and to enhance their susceptibility to chemotherapy drugs (Tsubaki et al., European Journal of Cancer 49: 3708-3717, 2013).

SLAMF1/CD150 (Signalling Lymphocytic Activation Molecule 1) is a type I glycoprotein belonging to the SLAM subfamily of the CD2-like family of proteins (Cocks, B. G. et al., Nature 376: 260-263 (1995)). SLAM F1 acts as a co-receptor that can modulate signaling via the tumour necrosis factor (TNF) family and antigen receptors (Dragovich & Mor, Autoimmunity Reviews 17(7): 674-682 (2018)).

The inventors have previously reported that (at least in humans) TLR4-mediated signalling via the TRAM pathway is initiated by an interaction between TRAM and SLAMF1. SLAMF1 was found to recruit TRAM to internalised TLR4 in the endosomal/phagosomal membrane. TRAM/SLAMF1 interaction domains were identified, constituting amino acids 68-95 of TRAM (the N-terminal region of the TRAM TIR domain) and the 15 C-terminal amino acids of SLAMF1 (Yurchenko et al., Journal of Cell Biology 217(4): 1411-1429 (2018)). Knockdown of SLAMF1 was found to inhibit TLR4-mediated IFNβ expression, and also to inhibit expression of IL-6 and TNF, along with the IFNβ-dependent cytokine CXCL10. Thus the interaction between TRAM and SLAM F1 was identified as a target to reduce cytokine expression in inflammatory conditions (Yurchenko et al., supra).

The inventors have now identified oligopeptidic compounds which inhibit the interaction between TRAM and SLAMF1, and which may be used for therapy of conditions associated with cytokine storm. The peptides are derived from the TRAM binding domain of SLAMF1, and comprise cell-penetrating sequences to mediate delivery to the cytosol, where the TRAM-SLAMF1 interaction occurs. As shown in the examples below, certain peptides were found to be particularly effective at inhibiting this interaction, causing downregulation of cytokines expressed as a result of signalling via the TRAM/SLAMF1 pathway, including IFNβ and TNF, and CXCL10, whose expression is upregulated by IFNβ (Yurchenko et al., supra).

Serendipitously, these SLAMF1-derived peptide compounds were also found to inhibit production of cytokines not associated with TRAM-mediated signalling from TLR4, including IL-1β (as shown in the Examples). It was subsequently determined that the peptide compounds unexpectedly inhibit formation of the Myddosome complex downstream of at least TLR4 and TLRs 7-9 (that is to say, the peptide compounds inhibit formation of Myddosome complexes which include Mal). As yet the mechanism for this inhibition has not been fully determined, but without being bound by theory, it is postulated that this may be achieved by binding of the peptide compounds to IRAK1 and/or IRAK4.

Blockade of TLR signalling using the peptide compounds of the invention can prevent or treat hypercytokinemia, including in the context of sepsis, thus fulfilling an important unmet therapeutic need. Given that signalling pathways associated with recognition of multiple types of pathogen can be targeted, the peptides of the invention can be used to prevent and treat hypercytokinemia associated with Gram-positive or Gram-negative bacterial infection, or viral infection. Moreover, signalling pathways through the Myddosome are known to be associated with several other disease conditions, including cancer and autoimmune diseases (Singer et al., Oncotarget 9(70): 33416-33439 (2018)), and thus the activity of the peptide compounds in blocking signalling through the Myddosome renders the compounds useful in therapy for these conditions.

In addition to effects on TLR signalling, peptide compounds of the invention have been shown directly to have anti-proliferative and/or cytotoxic effects on cancer cells.

### SUMMARY OF INVENTION

Accordingly, in a first aspect the invention provides an oligopeptidic compound comprising:
a) a first oligopeptidic component which:
   i) is capable of blocking signalling from TLR4, TLR7, TLR8 and/or TLR9; and/or
   ii) has an anti-proliferative and/or cytotoxic effect on cancer cells;
      and comprising the amino acid sequence set forth in SEQ ID NO: 1, or an amino acid sequence having at least 70% sequence identity thereto; and

b) a second oligopeptidic component, which is a cytosol-targeting cell penetrating oligopeptidic component.

In a second, related and more particular aspect the invention provides an oligopeptidic compound comprising:
a) a first oligopeptidic component capable of:
   i) binding TRAM, and inhibiting the interaction between TRAM and SLAMF1; and/or
   ii) inhibiting the formation of the Myddosome complex, said complex comprising Mal, MyD88, IRAK1 and IRAK4;
      and comprising the amino acid sequence set forth in SEQ ID NO: 1, or an amino acid sequence having at least 70% sequence identity thereto; and
b) a second oligopeptidic component, which is a cytosol-targeting cell penetrating oligopeptidic component.

In a third aspect, the invention provides a pharmaceutical composition comprising an oligopeptidic compound of the invention, and one or more pharmaceutically-acceptable diluents, carriers or excipients.

In a fourth aspect, the invention provides an oligopeptidic compound or composition of the invention for use in therapy.

In a fifth aspect, the invention provides an oligopeptidic compound or composition of the invention for use in treatment or prevention of a disease associated with hypercytokinemia in a human subject.

In a related sixth aspect, the invention provides an oligopeptidic compound of the invention, wherein the first oligopeptidic component of the oligopeptidic compound is capable of blocking signalling from TLR4, TLR7, TLR8 and/or TLR9, or a composition of the invention comprising such an oligopeptidic compound, for use in treatment or prevention of a disease associated with hypercytokinemia in a human subject.

In a seventh aspect, the invention provides an oligopeptidic compound or composition of the invention for use in treatment or prevention of an inflammatory disease, an autoimmune disease, cancer or ischemia-reperfusion injury.

In a related eighth aspect, the invention provides an oligopeptidic compound of the invention, wherein the first oligopeptidic component of the oligopeptidic compound is capable of blocking signalling from TLR4, TLR7, TLR8 and/or TLR9, or a composition of the invention comprising such an oligopeptidic compound, for use in treatment or prevention of an inflammatory disease, an autoimmune disease or ischemia-reperfusion injury.

In a ninth aspect, the invention provides an oligopeptidic compound of the invention, wherein the first oligopeptidic component of the oligopeptidic compound has an anti-proliferative and/or cytotoxic effect on cancer cells, or a composition of the invention comprising such an oligopeptidic compound, for use in the treatment of cancer.

Similarly, in a tenth aspect the invention provides a method of treating or preventing a disease in a human subject, comprising administering to the subject an oligopeptidic compound or a composition of the invention; wherein the disease is as defined in the fifth or seventh aspect.

In a related eleventh aspect the invention provides a method of treating or preventing a disease in a human subject, comprising administering to the subject an oligopeptidic compound of the invention or a composition of the invention; wherein the disease and oligopeptidic compound are as defined in the sixth, eighth or ninth aspect.

In a twelfth aspect, the invention also provides the use of an oligopeptidic compound of the invention in the manufacture of a medicament for use in the treatment or prevention of a disease in a human subject, wherein the disease is as defined in the fifth or seventh aspect.

In a related thirteenth aspect, the invention provides the use of an oligopeptidic compound of the invention in the manufacture of a medicament for use in the treatment or prevention of a disease in a human subject, wherein the disease and oligopeptidic compound are as defined in the sixth, eighth or ninth aspect.

In a fourteenth aspect, the invention provides a kit comprising an oligopeptidic compound or pharmaceutical composition of the invention, and a second therapeutically active agent.

In a fifteenth aspect, the invention provides a product comprising an oligopeptidic compound or pharmaceutical composition of the invention, and a second therapeutically active agent, as a combined preparation for separate, simultaneous or sequential use in the treatment or prevention of a disease in a human subject, wherein the disease is as defined in the fifth or seventh aspect.

In a related sixteenth aspect, the invention provides a product comprising an oligopeptidic compound of the invention or a pharmaceutical composition of the invention, and a second therapeutically active agent, as a combined preparation for separate, simultaneous or sequential use in the treatment or prevention of a disease in a human subject, wherein:
(i) the oligopeptidic compound and disease are as defined in the sixth or eighth aspect; or
(ii) the oligopeptidic compound and disease are as defined in the ninth aspect.

In a seventeenth aspect, the invention provides a nucleic acid molecule comprising a nucleotide sequence which encodes an oligopeptidic compound of the invention.

In an eighteenth aspect, the invention provides a construct comprising the nucleic acid molecule of the invention, or a vector comprising said construct or nucleic acid molecule.

In a nineteenth aspect, the invention provides a method of downregulating expression of interferon β by a human cell, comprising contacting the cell with an oligopeptidic compound of the second aspect.

In a related twentieth aspect, the invention provides a method of downregulating expression of interferon β by a human cell, comprising contacting the cell with an oligopeptidic compound of the first aspect, wherein the first oligopeptidic component of the oligopeptidic compound is capable of blocking signalling from TLR4, TLR7, TLR8 and/or TLR9.

### DETAILED DESCRIPTION OF THE INVENTION

The first and second aspects of the invention are directed to an oligopeptidic compound. As used herein, the term “oligopeptidic compound” means a compound which is composed of amino acids or equivalent subunits, which are linked together by peptide or equivalent bonds. Thus, the term “oligopeptidic compound” includes peptides and peptidomimetics.

By “equivalent subunit” is meant a subunit which is structurally and functionally similar to an amino acid. The backbone moiety of the subunit may differ from a standard amino acid, e.g. it may incorporate one or more nitrogen atoms instead of one or more carbon atoms. Generally speaking when an amino acid or amino acid residue is referred to herein, this would by analogy include an equivalent subunit, unless specified otherwise, or unless the context does not allow this. Thus references to amino acid sequences may include sequences of equivalent sub-units.

By “peptidomimetic” is meant a compound which is functionally equivalent or similar to a peptide and which can adopt a three-dimensional structure similar to its peptide counterparts, but which is not solely composed of amino acids linked by peptide bonds. A preferred class of peptidomimetics are peptoids, i.e. N-substituted glycines. Peptoids are closely related to their natural peptide counterparts, but they differ chemically in that their side chains are appended to nitrogen atoms along the molecule's backbone, rather than to the α-carbons as they are in amino acids.

The oligopeptidic compound of the invention comprises a first component, or part, and a second component, or part, e.g. a first peptide and a second peptide, as discussed further below. Thus, in an embodiment these parts of the oligopeptidic compound may be peptides (i.e. chains of amino acids joined by peptide bonds). In such an embodiment, the oligopeptidic compound may include further parts, in addition to the first and second peptides (e.g. a linker between the peptides). These further parts may also be peptides, or may be peptide equivalents as discussed above. In other embodiments the parts may be peptide equivalents and the entire compound may be a peptide equivalent.

Preferably, the oligopeptidic compound is an oligopeptide. The oligopeptidic compound may incorporate di-amino acids and/or β-amino acids. Most preferably, the oligopeptidic compound consists of α-amino acids.

An oligopeptide is a polymer formed from amino acids joined to one another by peptide bonds. As defined herein, an oligopeptide comprises at least three amino acids, though clearly an oligopeptidic compound according to the invention comprises more than three amino acids. In particular embodiments the oligopeptidic compound or oligopeptide of the invention comprises at least 15, 18, 20, 22 or 25 subunits, e.g. amino acids. An oligopeptidic compound or oligopeptide as defined herein has no particular maximum length, though typically the prefix “oligo” is used to designate a relatively small number of subunits. In particular embodiments the oligopeptidic compound or oligopeptide of the invention comprises a maximum of 50, 45, 40, 35 or 30 subunits, e.g. amino acids (clearly in the instance that the oligopeptidic compound of the invention is an oligopeptide all subunits are amino acids). In a particular embodiment the oligopeptidic compound or oligopeptide of the invention comprises 20-40 subunits (e.g. amino acids). In other embodiments the oligopeptidic compound or oligopeptide of the invention comprises 20-35, 20-30 or 25-35 subunits (e.g. amino acids).

The oligopeptidic compound according to the present invention may comprise only proteinogenic amino acids (i.e. the L-amino acids encoded by the standard genetic code). Alternatively the oligopeptidic compound according to the present invention may comprise one or more non-proteinogenic amino acids. For instance, the oligopeptidic compound according to the invention may comprise one or more D-amino acids, human-engineered amino acids or natural non-proteinogenic amino acids, e.g. amino acids formed through metabolic processes. Examples of non-proteinogenic amino acids which may be used include ornithine (a product of the urea cycle) and artificially-modified amino acids such as 9H-fluoren-9-ylmethoxycarbonyl (Fmoc)-, tert-Butyloxycarbonyl (Boc)-, and 2,2,5,7,8-pentamethylchromane-6-sulphonyl (Pmc)-protected amino acids, and amino acids having the carboxybenzyl (Z) group.

In vitro and/or in vivo stability of the oligopeptidic compounds of the invention may be improved or enhanced through the use of stabilising or protecting means known in the art, for example the addition of protecting or stabilising groups, incorporation of amino acid derivatives or analogues or chemical modification of amino acids, Such protecting or stabilising groups may for example be added at the N and/or C-terminus. An example of such a group is an acetyl group and other protecting groups or groups which might stabilise a peptide are known in the art.

A peptide consisting wholly of L-amino acids is known in the art as an L-peptide, while a peptide consisting wholly of D-amino acids is known in the art as a D-peptide. The term “inverso-peptide” is used to refer to a peptide with the same amino acid sequence as an L-peptide, but consisting wholly of D-amino acids (i.e. a D-peptide with the same sequence as a corresponding L-peptide). An inverso-peptide has a mirrored structure to its corresponding L-peptide (i.e. an L-peptide of the same amino acid sequence). Inverso-peptides can be advantageous for use in a clinical setting (relative to L-peptides) because they are not generally susceptible to degradation by serum proteases (due to their unnatural conformation inverso-peptides may not be recognised by protease enzymes). The oligopeptidic compound according to the invention may be an L-oligopeptide or a D-oligopeptide. Alternatively, the oligopeptidic compound of the invention may comprise both L-amino acids and D-amino acids (i.e. at least one L-amino acid and at least one D-amino acid).

As described above, the oligopeptidic compound of the invention comprises at least a first oligopeptidic component and a second oligopeptidic component. The first and second oligopeptidic components are two separate sequences of amino acids (or equivalent subunits) located within the oligopeptidic compound. Each oligopeptidic component is formed of a sequence of amino acids or equivalent subunits, which are linked together by peptide or equivalent bonds, as described above in the context of the term “oligopeptidic compound”. In a preferred embodiment, the first oligopeptidic component is a peptide. In another preferred embodiment, the second oligopeptidic component is a peptide (i.e. a cell-penetrating peptide, CPP). Most preferably, both the first and second oligopeptidic components are peptides.

According to the first aspect, the first oligopeptidic component is capable of blocking signalling from TLR4, TLR7, TLR8 and/or TLR9 and/or has an anti-proliferative and/or cytotoxic effect on cancer cells.

To block signaling from TLR4, TLR7, TLR8 or TLR9 does not require that the activity of the TLR4, TLR7, TLR8 or TLR9 protein itself is inhibited (though this is encompassed by the term). Rather, blockade of signaling from TLR4, TLR7, TLR8 and/or TLR9 simply requires that the first oligopeptidic component blocks or inhibits one or more signaling pathways downstream of TLR4, TLR7, TLR8 and/or TLR9 such that cellular effector functions initiated by TLR4, TLR7, TLR8 and/or TLR9 activation are blocked. As set out below, blockade of signaling from TLR4 may be achieved by, for instance, inhibiting the interaction between TRAM and SLAM F1. Blockade of signaling from TLR4, TLR7, TLR8 and TLR9 may be achieved by inhibiting the formation of the Myddosome complex comprising Mal, MyD88, IRAK1 and IRAK4. Any other means of blocking TLR4, TLR7, TLR8 and/or TLR9 signalling is also encompassed.

Signalling from TLR4 can be considered blocked if binding or exposure of TLR4 to a TLR4 ligand does not result in activation of one or more effector functions ordinarily stimulated by TLR4 activation. Similarly, signalling from TLR7 can be considered blocked if binding or exposure of TLR7 to a TLR7 ligand does not result in activation of one or more effector functions ordinarily stimulated by TLR7 activation; signalling from TLR8 can be considered blocked if binding or exposure of TLR8 to a TLR8 ligand does not result in activation of one or more effector functions ordinarily stimulated by TLR8 activation; signalling from TLR9 can be considered blocked if binding or exposure of TLR9 to a TLR9 ligand does not result in activation of one or more effector functions ordinarily stimulated by TLR9 activation.

The oligopeptidic compound may block signalling from a single one of TLR4, TLR7, TLR8 and TLR9. Alternatively the oligopeptidic compound may block signalling from a selection of TLR4, TLR7, TLR8 and TLR9, i.e. any two or three of TLR4, TLR7, TLR8 and TLR9. Preferably the oligopeptidic compound blocks signalling from at least TLR4. In a particular embodiment, the oligopeptidic compound blocks signalling from all four of TLR4, TLR7, TLR8 and TLR9.

Blockade of TLR4, TLR7, TLR8 and/or TLR9 signalling may be partial or complete, i.e. just one effector function downstream of the one or more TLRs may be inhibited by the first oligopeptidic component, or all effector functions, and the inhibited effector function(s) may be reduced or completely abrogated. Blockade of TLR4 signalling, for instance, may in particular result in the inhibition (i.e. reduction or abrogation) of the release of cytokines by an immune cell expressing TLR4 in response to exposure to a TLR4 ligand (e.g. LPS). Specifically, blockade of TLR4 signalling may result in the inhibition of the release of IFNβ, CXCL-10, TNF, IL-1β and/or IL-6. Similarly, blockade of TLR7 signalling may in particular result in the inhibition (i.e. reduction or abrogation) of the release of cytokines by an immune cell expressing TLR7 in response to exposure to a TLR7 ligand (e.g. imiquimod). Specifically, blockade of TLR7 signalling may result in the inhibition of the release of IFNβ, Blockade of TLR8 signalling may in particular result in the inhibition (i.e. reduction or abrogation) of the release of cytokines by an immune cell expressing TLR8 in response to exposure to a TLR8 ligand (e.g. CL075). Specifically, blockade of TLR8 signalling may result in the inhibition of the release of IFNβ. Blockade of TLR9 signalling may in particular result in the inhibition (i.e. reduction or abrogation) of the release of cytokines by an immune cell expressing TLR9 in response to exposure to a TLR9 ligand (e.g. a CpG oligonucleotide such as CpG 2006 ODN). Blockade of TLR9 signalling may in particular result in the inhibition of the release of IFNβ, CXCL10, TNF and/or IL-1β.

Inhibition of the release of one or more of these cytokines may be assessed and identified by standard methods in the art, e.g. ELISA as demonstrated in the Examples below. Immune cells (such as macrophages) may be contacted with an oligopeptidic compound of interest and a TLR4, TLR7, TLR8 or TLR9 ligand (as appropriate), and the resulting levels of expression of relevant cytokines (e.g. IFNβ, CXCL-10, TNF, IL-1β and/or IL-6) compared to the levels of expression demonstrated by control cells contacted only with the TLR4, TLR7, TLR8 or TLR9 ligand.

For reference, human IFNβ has the UniProt accession number P01574, human CXCL-10 has the UniProt accession number P02778, human TNF has the UniProt accession number P01375, human IL-1β has the UniProt accession number P01584 and human IL-6 has the UniProt accession number P05231.

Alternatively or additionally to blocking TLR4, TLR7, TLR8 and/or TLR9 signalling, the oligopeptidic compound of the invention has an anti-proliferative and/or cytotoxic effect on cancer cells. An “anti-proliferative effect” as used herein means that the oligopeptidic compound, when applied to cancer cells (e.g. a cancer cell line or primary cancer cells) prevents the cancer cells from proliferating (or dividing), or reduces their proliferation. The term “anti-proliferative” can thus be seen as equivalent to “cytostatic”, and the two terms are used interchangeably herein. The skilled person may straightforwardly determine whether an oligopeptidic compound of interest has an anti-proliferative effect on cancer cells. This may in particular be achieved by applying the compound of interest to a culture of cancer cells in vitro or ex vivo (using a cancer cell line or isolated primary cancer cells) and comparing the proliferation of the cancer cells with a control culture of the cancer cells to which the compound of interest is not applied. If the compound of interest has an anti-proliferative effect, the cells to which the compound was applied will have proliferated less than the cells not contacted with the compound, and may preferably not have proliferated at all.

Rather than simply preventing the proliferation of cancer cells, the oligopeptidic compound of the invention may instead (or additionally) kill the cells. That is to say, the oligopeptidic compound may be cytotoxic towards cancer cells (i.e. have a cytotoxic effect on cancer cells). The term “cytotoxic” has herein its standard meaning in the art, that is to say that it refers to a compound which is toxic to cells, thus causing their death. A “cytotoxic effect” as used herein thus means that the oligopeptidic compound, when applied to cancer cells (e.g. a cancer cell line or primary cancer cells) causes the death of some or all of the cells to which it is applied. The skilled person may straightforwardly determine whether an oligopeptidic compound of interest has a cytotoxic effect on cancer cells. This may in particular be achieved by applying the compound of interest to a culture of cancer cells in vitro or ex vivo (using a cancer cell line or isolated primary cancer cells) and analysing the impact of the compound of interest on cancer cell number. If the compound of interest is cytotoxic to the cancer cells to which it is applied, the cells to which it is applied should be killed, or at least the number of viable cells should be reduced below the number originally seeded, whereas in a control culture of the cancer cells (not contacted with the compound of interest) the number of viable cells would be expected to increase during culture.

Methods by which the effect of a compound of interest on cancer cells can be assessed (e.g. to identify any cytostatic or cytotoxic effect) are described in more detail in the examples. Preferably, the oligopeptidic compound of the invention is selectively cytostatic or cytotoxic towards cancer cells, i.e. it is cytostatic and/or cytotoxic towards cancer cells, but is not cytostatic or cytotoxic towards healthy (i.e. non-cancerous) cells, or at least has a much greater cytostatic and/or cytotoxic effect on cancer cells than on healthy cells.

The oligopeptidic compound of the invention may have an anti-proliferative or cytotoxic effect on any type of cancer cell, including any cancer cell line or primary cancer cell. Generally to determine whether a compound of interest is cytostatic or cytotoxic towards primary cancer cells the cells are removed from the body of the person or animal in which the cancer is present and cultured ex vivo to be tested with the oligopeptidic compound of interest, as described above. The primary cancer cells may be derived from any suitable source, e.g. a human cancer patient or a laboratory animal being used in investigation of a cancer model. Commonly the oligopeptidic compound has an anti-proliferative or cytotoxic effect on a human cancer cell.

In a preferred embodiment, the oligopeptidic compound of the invention has an anti-proliferative or cytotoxic effect on multiple myeloma cells. The multiple myeloma cells may be a multiple myeloma cell line or primary multiple myeloma cells. For instance the oligopeptidic compound of the invention may have a cytostatic or cytotoxic effect on multiple myeloma cells from the cell lines INA6, JJN3 and/or ANBL6. Preferably the oligopeptidic compound of the invention has a cytostatic or cytotoxic effect on the ANBL6 multiple myeloma cell line. The oligopeptidic compound of the invention may have a cytostatic or cytotoxic effect on primary VK-MYC myeloma cells, that is to say model myeloma cells derived from the Vk*MYC mouse model of multiple myeloma. Vk*MYC mice are transgenic mice in which the MYC gene has been placed under the control of the kappa light chain gene regulatory elements, resulting in sporadic activation of MYC in germinal centre (GC) B cells. As a result Vk*MYC mice universally develop post-GC plasmacytoma (PC) tumours which fully recapitulate the biological and clinical features of human multiple myeloma (Chesi et al., Cancer Cell 13: 167-180, 2008).

The oligopeptidic compounds of the invention may be both capable of blocking signaling from TLR4, TLR7, TLR8 and/or TLR9, and have an anti-proliferative and/or cytotoxic effect on cancer cells. However, as shown in the Examples, the oligopeptidic compounds of the invention may display only one of these characteristics.

Alternatively considered, and as set out in the second aspect of the invention above, the first oligopeptidic component may be capable of binding to TRAM, and inhibiting its interaction with SLAMF1; and/or the first oligopeptidic component may be capable of inhibiting the formation of the Myddosome complex, which comprises Mal, MyD88, IRAK1 and IRAK4. Throughout the present disclosure, all references to proteins refer to human proteins, unless explicitly stated otherwise. Thus the first oligopeptidic component may be capable of binding to human TRAM and inhibiting its interaction with human SLAMF1, and/or inhibiting the formation of the Myddosome complex in human cells. For reference, human TRAM has the UniProt accession number Q86XR7; human SLAMF1 has the UniProt accession number Q13291; human Mal has the UniProt accession number P58753; human MyD88 has the UniProt accession number Q99836; human IRAK1 has the UniProt accession number P51617; human IRAK4 has the UniProt accession number Q9NWZ3.

The term “interaction” as defined herein refers to the contacting or binding of two proteins to each other. Inhibition of an interaction between two proteins thus prevents the two proteins from binding or contacting one another, and thus also inhibits any downstream effects resulting from the interaction. As noted above, the first oligopeptidic component may be capable of binding TRAM and inhibiting its interaction with SLAMF1. In this case, the first oligopeptidic component prevents TRAM from binding SLAMF1. The effect of the inhibition of this interaction between the two proteins is the inhibition of downstream cytokine production, e.g. the inhibition of expression of IFNβ, TNF and CXCL10.

It is straightforward for the skilled person to determine whether a particular putative first oligopeptidic component (e.g. peptide) binds to TRAM. Binding of a putative first oligopeptidic component to TRAM can be assessed by e.g. co-precipitation experiments: if a putative first oligopeptidic component co-precipitates with TRAM from a solution (e.g. a cell lysate) containing TRAM, that oligopeptidic component can be considered to bind TRAM. Such a co-precipitation experiment may in particular be performed by immunoprecipitation. For example, a cell lysate comprising TRAM can be obtained, a putative first oligopeptidic component added to the lysate, and co-precipitation performed to determine whether the oligopeptidic component binds to TRAM. Such co-precipitation may be aided by conjugation of a tag to the oligopeptidic component of interest which can be recognised by an antibody. Suitable tags include e.g. a FLAG or HA tag, or an entire protein such as GFP. Alternatively, binding of an oligopeptidic component of interest to TRAM may be tested by other methods well-known in the art, e.g. ELISA, far Western blot, etc. It is straightforward for the skilled person to determine whether an oligopeptidic component of interest binds to TRAM.

An oligopeptidic component (e.g. peptide) that has been determined to bind to TRAM can also be easily analysed by the skilled person to determine whether it inhibits the interactions of the protein with SLAMF1. The interactions between TRAM and SLAMF1 can be easily checked by co-precipitation, e.g. immunoprecipitation. An oligopeptidic component which eliminates co-precipitation of binding partners from solution inhibits the binding of the proteins to each other.

For instance, a cell lysate containing TRAM and SLAM F1 may be obtained and an oligopeptidic component of interest added. Such a cell lysate may be obtained from cells that natively express both proteins (e.g. macrophages) or from a cell line transfected or transduced to express both proteins. HEK293 is an example of a suitable cell line. Alternatively, rather than adding an oligopeptidic component of interest to a cell lysate, a putative oligopeptidic compound of the invention comprising the oligopeptidic component of interest may be applied to the cells prior to lysis. If addition of the oligopeptidic compound eliminates or reduces co-precipitation of TRAM and SLAM F1 in comparison to a control co-precipitation from a corresponding lysate to which the oligopeptidic compound of interest was not added, or application of the putative oligopeptidic compound of the invention comprising the first oligopeptidic component of interest eliminates or reduces co-precipitation of TRAM and SLAMF1 in comparison to a control culture to which the putative oligopeptidic compound of the invention was not applied, the oligopeptidic component of interest can be considered to inhibit the interaction between TRAM and SLAMF1. Testing of oligopeptidic components for inhibition of the TRAM-SLAMF1 interaction is demonstrated in the Examples. Alternative methods for identifying inhibition of protein-protein interactions are also known to the skilled person and may be used as appropriate.

As detailed above, the first oligopeptidic component may inhibit the formation of the Myddosome complex comprising Mal, MyD88, IRAK1 and IRAK4. Throughout this section (i.e. the detailed description), for conciseness the term “Myddosome complex” is used to refer to the Myddosome complex comprising Mal, MyD88, IRAK1 and IRAK4, such as the TLR4 Myddosome complex. By inhibition of the formation of the Myddosome complex is meant that the first oligopeptidic component prevents or reduces formation of the Myddosome complex, i.e. it prevents or reduces the formation of the complex between Mal, MyD88, IRAK1 and IRAK4. This may be effected by preventing or reducing the recruitment of any one or more of the four proteins to the complex. As detailed above, upon TLR activation Mal recruits MyD88, which in turn recruits IRAK4, which itself in turn recruits IRAK1. Formation of the complex may be inhibited at any stage. For instance, inhibition of the final stage of complex formation (i.e. blocking the recruitment if IRAK1 by IRAK4) inhibits the formation of the Myddosome, even though a complex could still be formed between Mal, MyD88 and IRAK4. Complete formation of the Myddosome is required for its signalling activity to occur, and thus its downstream effector functions to take place. In other words, inhibition of the formation of the Myddosome may be achieved by inhibiting the complete formation of the Myddosome, without inhibiting all interactions between members of the Myddosome.

Alternatively, inhibition of the formation of the Myddosome may be achieved by blocking all interactions between members of the Myddosome. As shown in the Examples below, oligopeptidic compounds of the present invention may be capable of preventing the formation of any interactions between members of the Myddosome. As shown, the oligopeptidic compounds may block the formation of interactions between MyD88 and IRAK4, MyD88 and IRAK1, IRAK4 and IRAK1, Mal and IRAK1, and Mal and MyD88. Thus formation of the Myddosome complex may be entirely blocked.

Inhibition of the formation of this complex can be tested broadly as described above, i.e. by co-precipitation experiments, in particular by co-immunoprecipitation. As detailed above, activation of TLR signalling (via e.g. TLR4) in cells of the innate immune system, e.g. macrophages, naturally initiates formation of the Myddosome. If an innate immune cell such as a macrophage is activated via TLR4 (or any other suitable TLR, as listed above) and lysed, the members of the Myddosome complex co-precipitate. An oligopeptidic component that inhibits formation of the complex can be identified based on its ability to prevent co-precipitation of members of the Myddosome complex, as demonstrated in the Examples below. In particular, as shown in the Examples, a putative oligopeptidic compound of the invention comprising a cell penetrating oligopeptidic component and a first oligopeptidic component of interest may be applied to macrophages (or other appropriate immune cells), to which a TLR4 ligand is subsequently applied. The macrophages may then be lysed and co-precipitation of Myddosome components performed. If the members of the Myddosome complex do not co-precipitate from the macrophages pre-treated with the oligopeptidic compound of interest, this indicates that the putative first oligopeptidic component inhibits formation of the Myddosome complex. Specifically, cells pre-treated with the putative oligopeptidic compound of the invention may be compared to non-pre-treated control cells. If interactions between members of the Myddosome complex are seen in the control cells that are not seen in the pre-treated cells, this demonstrates that the putative first oligopeptidic component inhibits formation of the Myddosome. Conversely, if members of the Myddosome complex co-precipitate as they do in non-pre-treated cells, such that administration of the putative oligopeptidic compound of the invention has no impact on co-precipitation relative to non-pre-treated control cells, this indicates that the putative first oligopeptidic component does not inhibit the formation of the Myddosome complex. Thus whether a putative first oligopeptidic component has the requisite activity may be easily determined using standard techniques in the art.

In addition to having one or both of the activities described above, the first oligopeptidic component comprises the amino acid sequence set forth in SEQ ID NO: 1, or an amino acid sequence having at least 70% sequence identity thereto. SEQ ID NO: 1 corresponds to the peptide referred to in the Examples as Peptide-7 (abbreviated to Pep-7 or P7), and is derived from the C-terminus of SLAMF1. Peptide-7 corresponds to amino acids 324-333 of SLAMF1, with a proline to threonine substitution at position 10 (i.e. proline corresponding to Pro333 of SLAMF1 is replaced with threonine). This proline to threonine substitution was found to enhance binding of the peptide to TRAM (as shown in the Examples).

The first oligopeptidic component may consist of the amino acid sequence set forth in SEQ ID NO: 1, or an amino acid sequence having at least 70% sequence identity thereto. The first oligopeptidic component may alternatively comprise or consist of an amino acid sequence having at least 75%, 80%, 85% or 90% sequence identity to SEQ ID NO: 1. The first oligopeptidic component does not comprise the entire SLAMF1 protein.

It is advantageous that the size of the oligopeptidic compound of the invention is kept to a minimum, e.g. for ease of synthesis, though it is also necessary that its components are of sufficient length for effective activity. It is also advantageous that the length of the first oligopeptidic component is minimised in order to prevent undesired secondary structure forming, which may negatively impact upon target binding. Accordingly, the first oligopeptidic component may in certain embodiments be a maximum of 20, 19, 18, 17, 16, 15, 14, 13, 12 or 11 amino acids (or, throughout, equivalent subunits) long. The first oligopeptidic component may in certain embodiments be a minimum of 7, 8 or 9 amino acids long. The first peptide may have a length in the range of 7-15 amino acids, 8-13 amino acids, 8-12 amino acids or 9 to 11 amino acids. In particular embodiments the first peptide is 10, 11,12 or 13 amino acids long.

As noted above, the first oligopeptidic component comprises an amino acid sequence having at least 70% sequence identity to SEQ ID NO: 1. SEQ ID NO: 1 is 10 amino acids in length, and thus as is apparent from the above paragraph the first oligopeptidic component may be an lengthened or shortened relative to SEQ ID NO: 1. SEQ ID NO: 1 may by lengthened by amino acid insertions (i.e. amino acids inserted within its existing sequence) and/or amino acid extensions (i.e. additional amino acids located at the N- and/or C-terminus of the peptide). SEQ ID NO: 1 may be shortened by amino acid deletions, including truncations from one or both ends, and/or internal deletions. Clearly when amino acid insertions or deletions are made, this is subject to the provisions regarding sequence identity to SEQ ID NO: 1.

However, as shown in the Examples, it has been found that extending SEQ ID NO: 1 at its C-terminus appears to reduce target binding and thus efficacy. Accordingly, in a particular embodiment, the first oligopeptidic component does not comprise a C-terminal extension relative to SEQ ID NO: 1. By “does not comprise a C-terminal extension relative to SEQ ID NO: 1” it is meant that the C-terminal amino acid of the first oligopeptidic component is the C-terminal amino acid of SEQ ID NO: 1, an amino acid equivalent to the C-terminal amino acid of SEQ ID NO: 1, or, if the C-terminal amino acid of SEQ ID NO: 1 has been deleted, the most C-terminal amino acid remaining from SEQ ID NO: 1 (or its equivalent). An amino acid equivalent to the C-terminal amino acid of SEQ ID NO: 1 is present in variants of SEQ ID NO: 1 in which the amino acid (threonine) at position 10 of SEQ ID NO: 1 has been substituted. In such variants of SEQ ID NO: 1, the amino acid which has been substituted for the C-terminal threonine of SEQ ID NO: 1 is equivalent to the C-terminal amino acid of SEQ ID NO: 1. Similarly, if the C-terminal threonine of SEQ ID NO: 1 has been deleted, and the most C-terminal remaining amino acid (e.g. the leucine at position 9) has been substituted, then the amino acid that has replaced the most C-terminal remaining amino acid is equivalent to that amino acid.

On the other hand, the Examples show that the peptide of SEQ ID NO: 1 can be extended at its N-terminus, e.g. by up to 3 amino acids, without substantially impacting on target binding. Accordingly, in an embodiment the oligopeptidic component comprises an N-terminal extension of up to 3 amino acids relative to SEQ ID NO: 1. In particular embodiments the oligopeptidic component comprises an N-terminal extension of 1 amino acid, 2 amino acids or 3 amino acids relative to SEQ ID NO: 1. As detailed above, an N-terminal extension of the first oligopeptidic component is formed by the addition of amino acids to the first oligopeptidic component, N-terminal to the isoleucine residue at position 1 of SEQ ID NO: 1, or the equivalent residue. In accordance with the paragraph above, if the isoleucine residue at position 1 of SEQ ID NO: 1 has been substituted, the amino acid which has been substituted for the N-terminal isoleucine of SEQ ID NO: 1 is equivalent to this residue.

In the discussion below reference is made to first and second peptides, but it will be understood that this extends by analogy to first and second oligopeptidic components, and to amino acid sequences made of equivalent subunits.

If the first peptide has an N-terminal extension relative to SEQ ID NO: 1, any amino acids may constitute the extension. However, it is preferred that any N-terminal extension is constituted of the amino acids N-terminal to SEQ ID NO: 1 at its native location within SLAMF1. As detailed above, SEQ ID NO: 1 is derived from amino acids 324-333 of SLAMF1. Thus it is preferred that an extension of up to 3 amino acids is based on the amino acids present at positions 321-323 of SLAMF1. Positions 321-323 of SLAMF1 have the sequence Thr-Asn-Ser. Accordingly, if the first peptide comprises an N-terminal extension of a single amino acid relative to SEQ ID NO: 1, it is preferred that the single amino acid of the extension (which forms the N-terminus of the first peptide) is serine; if the first peptide comprises an N-terminal extension of two amino acids relative to SEQ ID NO: 1, it is preferred that the N-terminal extension has the amino acid sequence Asn-Ser; if the first peptide comprises an N-terminal extension of three amino acids relative to SEQ ID NO: 1, it is preferred that the N-terminal extension has the amino acid sequence Thr-Asn-Ser.

Thus, in a particular embodiment, the first peptide comprises the amino acid sequence of SEQ ID NO: 1 with an N-terminal extension of a single serine residue. Such an amino acid sequence is set forth in SEQ ID NO: 16, and a peptide of this sequence is referred to herein as P3sh3. Thus the first peptide may comprise or consist of the amino acid sequence set forth in SEQ ID NO: 16. In another embodiment, the first peptide comprises the amino acid sequence of SEQ ID NO: 1 with an N-terminal extension of two amino acids, with the sequence Asn-Ser. Such an amino acid sequence is set forth in SEQ ID NO: 6 (referred to herein as P3sh2). Thus the first peptide may comprise or consist of the amino acid sequence set forth in SEQ ID NO: 6. In another embodiment, the first peptide comprises the amino acid sequence of SEQ ID NO: 1 with an N-terminal extension of three amino acids, with the sequence Thr-Asn-Ser. Such an amino acid sequence is set forth in SEQ ID NO: 5 (referred to herein as P3sh1). Thus the first peptide may comprise or consist of the amino acid sequence set forth in SEQ ID NO: 5.

In an alternative embodiment, the first peptide does not comprise an N-terminal extension relative to SEQ ID NO: 1. The term “does not comprise an N-terminal extension relative to SEQ ID NO: 1” is equivalent to the term “does not comprise a C-terminal extension relative to SEQ ID NO: 1” defined above, but in relation to the N-terminus rather than the C-terminus. Thus the term “does not comprise an N-terminal extension relative to SEQ ID NO: 1” means that the N-terminal amino acid of the first peptide is the N-terminal amino acid of SEQ ID NO: 1, an amino acid equivalent to the N-terminal amino acid of SEQ ID NO: 1, or, if the N-terminal amino acid of SEQ ID NO: 1 has been deleted, the most N-terminal amino acid remaining from SEQ ID NO: 1 (or its equivalent). An amino acid equivalent to the N-terminal amino acid of SEQ ID NO: 1 is present in variants of SEQ ID NO: 1 in which the amino acid (isoleucine) at position 1 of SEQ ID NO: 1 has been substituted. In such variants of SEQ ID NO: 1, the amino acid which has been substituted for the N-terminal isoleucine of SEQ ID NO: 1 is equivalent to the N-terminal amino acid of SEQ ID NO: 1. Similarly, if the N-terminal isoleucine of SEQ ID NO: 1 has been deleted, and the most N-terminal remaining amino acid (e.g. the threonine at position 2) has been substituted, then the amino acid that has replaced the most N-terminal remaining amino acid is equivalent to that amino acid.

As set out above, the first peptide comprises the amino acid sequence set forth in SEQ ID NO: 1 or a variant thereof. A variant of SEQ ID NO: 1, as defined herein, is an amino acid sequence which is different to SEQ ID NO: 1 but which has at least 70% sequence identity to SEQ ID NO: 1. A variant of SEQ ID NO: 1 can be obtained by amino acid insertion, deletion or substitution within SEQ ID NO: 1, or any combination thereof. Amino acids may be inserted, deleted or substituted at any position within SEQ ID NO: 1.

In a particular embodiment, the first peptide is a variant of SEQ ID NO: 1 in which the amino acid at the position corresponding to position 10 of SEQ ID NO: 1 is not proline, i.e. is any amino acid other than proline. In this embodiment, the amino acid at the position corresponding to position 10 of SEQ ID NO: 1 is preferably threonine, asparagine or arginine, most preferably threonine. Thus in this embodiment, the first peptide comprises the amino acid sequence set forth in SEQ ID NO: 1, or an amino acid sequence having at least 70% sequence identity to SEQ ID NO: 1 and in which the amino acid at the position corresponding to position 10 of SEQ ID NO: 1 is not proline.

As mentioned above, a variant of SEQ ID NO: 1 as defined herein is an amino acid sequence having at least 70% sequence identity to SEQ ID NO: 1. Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32(5):1792-1797, 2004) may be used for multiple sequence alignments, though any other appropriate programme may be used. Another suitable alignment programme is BLAST, using the blastp algorithm for protein alignments. Whether the alignment is pairwise or multiple, it must be performed globally (i.e. across the entirety of the reference sequence) rather than locally.

Sequence alignments and percentage identity calculations may be determined using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

In a particular embodiment, the first peptide comprises a sequence having 1 to 3 amino acid substitutions relative to SEQ ID NO: 1. As shown in the Examples, substitutions at certain positions within SEQ ID NO: 1 have been shown not to negatively impact its efficacy. In particular, substitutions at positions 2, 4 and 10 have been found not to have a negative impact upon target binding. Accordingly, it is preferred that the 1 to 3 substitutions relative to SEQ ID NO: 1 are located at positions 2, 4 and/or 10 of SEQ ID NO: 1, or positions equivalent thereto. In particular embodiments, the first peptide comprises or consists of an amino acid sequence having a single amino acid substitution relative to SEQ ID NO: 1 at position 2 of SEQ ID NO: 1, at position 4 of SEQ ID NO: 1 or at position 10 of SEQ ID NO: 1. In other embodiments, the first peptide comprises or consists of an amino acid sequence having two amino acid substitutions relative to SEQ ID NO: 1, at positions 2 and 4 of SEQ ID NO: 1, at positions 2 and 10 of SEQ ID NO: 1 or at positions 4 and 10 of SEQ ID NO: 1. In another embodiment, the first peptide comprises or consists of an amino acid sequence having three amino acid substitutions relative to SEQ ID NO: 1, at positions 2, 4 and 10 of SEQ ID NO: 1. In all the aforementioned embodiments of this paragraph, it is preferred that no additional sequence modifications relative to SEQ ID NO: 1 (i.e. amino acid insertions or deletions) are present. The substitutions at positions 2, 4 and 10 described above may be for any alternative amino acid to those present at those positions in SEQ ID NO: 1.

As shown in the Examples particular substitutions at positions 2, 4 and 10 have been found to be particularly well tolerated (in that target binding is not negatively impacted, or potentially even improved, or that the peptide's anti-proliferative or cytotoxic effect on cancer cells is expanded and/or enhanced). In particular, substitution of the threonine residue at position 2 of SEQ ID NO: 1 for an asparagine residue, an aspartic acid residue, a histidine residue or a lysine residue has been found to be particularly well tolerated. In particular, substitution of the threonine residue at position 2 of SEQ ID NO: 1 for an asparagine, aspartic acid or histidine residue has been found to be well tolerated in that the peptide's ability to block signalling from TLR4 is retained. Substitution of the threonine residue at position 2 of SEQ ID NO: 1 for a lysine residue has been found to substantially enhance the anti-proliferative/cytotoxic activity of the peptide towards cancer cells.

Similarly, substitution of the tyrosine residue at position 4 of SEQ ID NO: 1 for an alanine residue, a phenylalanine residue, an asparagine residue, an aspartic acid residue or a serine residue has been found to be particularly well tolerated. In particular, substitution of the tyrosine residue at position 4 of SEQ ID NO: 1 for a phenylalanine residue has been found to be well tolerated in that the peptide's ability to block signalling from TLR4 is retained. Substitution of the tyrosine residue at position 4 of SEQ ID NO: 1 for an asparagine, aspartic acid or serine residue has been found to substantially enhance the anti-proliferative/cytotoxic activity of the peptide towards cancer cells. Substitution of the tyrosine residue at position 4 of SEQ ID NO: 1 for an alanine residue has been found to provide a peptide in which the ability to block signalling from TLR4 is retained and its anti-proliferative/cytotoxic activity towards cancer cells is substantially increased.

Additionally, as noted above, the amino acid at position 324 of SLAM F1 (equivalent to position 10 of SEQ ID NO: 1) is a proline. Inclusion of a threonine residue at this position has been found to be advantageous, but substitution of the threonine at position 10 of SEQ ID NO: 1 for a proline residue (as is found in the native SLAMF1 sequence) is also well tolerated. Thus, the following are particularly preferred substitutions in SEQ ID NO: 1: Thr2Asn, Thr2Asp, Thr2His, Thr2Lys, Tyr4Ala, Tyr4Phe, Tyr4Asn, Tyr4Asp, Tyr4Ser, Thr10Asp, Thr10Arg and Thr10Pro.

Accordingly, in particular embodiments the first peptide comprises 1 to 3 amino acid substitutions relative to SEQ ID NO: 1, selected from substitution of the threonine residue at position 2 of SEQ ID NO: 1 for an asparagine residue (Thr2Asn), an aspartic acid residue (Thr2Asp), a histidine residue (Thr2His) or a lysine residue (Thr2Lys); substitution of the tyrosine residue at position 4 of SEQ ID NO: 1 for an alanine residue (Tyr4Ala), a phenylalanine residue (Tyr4Phe), an asparagine residue (Tyr4Asn), an aspartic acid residue (Tyr4Asp) or a serine residue (Tyr4Ser); and substitution of the threonine at position 10 of SEQ ID NO: 1 for a proline residue (Thr10Pro), an asparagine residue (Thr10Asn) or an arginine residue (Thr10Arg). In particular embodiments, the first peptide comprises or consists of an amino acid sequence having a single amino acid substitution relative to SEQ ID NO: 1, the single amino acid substitution selected from Thr2Asn, Thr2Asp, Thr2His, Thr2Lys, Tyr4Ala, Tyr4Phe, Tyr4Asn, Tyr4Asp, Tyr4Ser, Thr10Asn, Thr10Arg and Thr10Pro. In other embodiments, the first peptide comprises or consists of an amino acid sequence having two or three amino acid substitutions relative to SEQ ID NO: 1, selected from those recited above.

The peptide of SEQ ID NO: 2 corresponds to that of SEQ ID NO: 1 comprising the Thr10Pro substitution. In other words the peptide of SEQ ID NO: 2 has the amino acid sequence of SLAMF1 positions 324-333. The peptide of SEQ ID NO: 2 is referred to in the Examples as Peptide-6 (Pep-6 or P6). In a particular embodiment, the first peptide comprises or consists of the amino acid sequence set forth in SEQ ID NO: 2. The peptide of SEQ ID NO: 3 corresponds to that of SEQ ID NO: 1 comprising the Tyr4Ala substitution. The peptide of SEQ ID NO: 3 is referred to in the Examples as P7-A4. In a particular embodiment the first peptide comprises or consists of the amino acid sequence set forth in SEQ ID NO: 3. The peptide of SEQ ID NO: 4 corresponds to that of SEQ ID NO: 1 comprising the Thr2Asn substitution. The peptide of SEQ ID NO: 4 is referred to in the Examples as P7-N2. In a particular embodiment the first peptide comprises or consists of the amino acid sequence set forth in SEQ ID NO: 4.

The peptide of SEQ ID NO: 17 corresponds to that of SEQ ID NO: 1 comprising the Thr2Asp substitution. The peptide of SEQ ID NO: 17 is referred to in the Examples as P7-D2. In a particular embodiment the first peptide comprises or consists of the amino acid sequence set forth in SEQ ID NO: 17. The peptide of SEQ ID NO: 18 corresponds to that of SEQ ID NO: 1 comprising the Thr2His substitution. The peptide of SEQ ID NO: 18 is referred to in the Examples as P7-H2. In a particular embodiment the first peptide comprises or consists of the amino acid sequence set forth in SEQ ID NO: 18. The peptide of SEQ ID NO: 19 corresponds to that of SEQ ID NO: 1 comprising the Tyr4Phe substitution. The peptide of SEQ ID NO: 19 is referred to in the Examples as P7-F4. In a particular embodiment the first peptide comprises or consists of the amino acid sequence set forth in SEQ ID NO: 19. The peptide of SEQ ID NO: 20 corresponds to that of SEQ ID NO: 1 comprising the Thr10Arg substitution. The peptide of SEQ ID NO: 20 is referred to in the Examples as P7-R10. In a particular embodiment the first peptide comprises or consists of the amino acid sequence set forth in SEQ ID NO: 20. The peptide of SEQ ID NO: 104 corresponds to that of SEQ ID NO: 1 comprising the Thr10Asn substitution. The peptide of SEQ ID NO: 104 is referred to in the Examples as P7-N10. In a particular embodiment the first peptide comprises or consists of the amino acid sequence set forth in SEQ ID NO: 104.

The peptide of SEQ ID NO: 118 corresponds to that of SEQ ID NO: 1 comprising the Thr2Lys substitution. The peptide of SEQ ID NO: 118 is referred to in the Examples as P7-K2. In a particular embodiment the first peptide comprises or consists of the amino acid sequence set forth in SEQ ID NO: 118. The peptide of SEQ ID NO: 119 corresponds to that of SEQ ID NO: 1 comprising the Tyr4Asn substitution. The peptide of SEQ ID NO: 119 is referred to in the Examples as P7-N4. In a particular embodiment the first peptide comprises or consists of the amino acid sequence set forth in SEQ ID NO: 119. The peptide of SEQ ID NO: 120 corresponds to that of SEQ ID NO: 1 comprising the Tyr4Asp substitution. The peptide of SEQ ID NO: 120 is referred to in the Examples as P7-D4. In a particular embodiment the first peptide comprises or consists of the amino acid sequence set forth in SEQ ID NO: 120. The peptide of SEQ ID NO: 121 corresponds to that of SEQ ID NO: 1 comprising the Tyr4Ser substitution. The peptide of SEQ ID NO: 121 is referred to in the Examples as P7-S4. In a particular embodiment the first peptide comprises or consists of the amino acid sequence set forth in SEQ ID NO: 121.

As detailed above, the oligopeptidic compound of the invention also comprises a second oligopeptidic component, which is a cytosol-targeting cell penetrating oligopeptidic component, referred to hereinafter for ease as a cell-penetrating peptide (CPP). The binding targets of the first oligopeptidic component, including TRAM, are all located in the cell cytosol, and so delivery of the oligopeptidic compound to the cytosol is required in order for the oligopeptidic compound to have the required effects. This delivery is achieved by the inclusion of a cytosol-targeting cell penetrating peptide (CPP).

A wide variety of cytosol-targeting CPPs are known and available. Cell penetrating peptides may vary greatly in size, sequence and charge, and indeed in their mechanism of function (which is presently not known for some peptides and not fully elucidated for others), but share the common ability to translocate across the plasma membrane and deliver an attached or associated moiety (the so-called “cargo”) into the cytoplasm of a cell.

Whilst CPPs are not characterised by a single structural or functional motif, tools to identify CPPs are available and the skilled person can readily determine whether a peptide sequence may function to facilitate the uptake of the peptide of which it forms a domain, i.e. whether a domain may function as an uptake (import) peptide, e.g. a CPP. For example, Hansen et al. (Predicting cell-penetrating peptides, Advanced Drug Delivery Reviews, 2008, 60, pp. 572-579), provides a review of methods for CPP prediction based on the use of principal component analysis (“z-predictors”) and corresponding algorithms based on original work by Hällbrink et al. (Prediction of Cell-Penetrating Peptides, International Journal of Peptide Research and Therapeutics, 2005, 11(4), pp. 249-259). In brief, the methodology works by computing z-scores of a candidate peptide as based on a numerical value and an associate range. If the z-scores fall within the range of known CPP z-scores, the examined peptides are classified as CPPs. The method was shown to have high accuracy (about 95% prediction of known CPPs).

Additional methods for the prediction of CPPs have been developed subsequently (see e.g. Sanders et al., Prediction of Cell Penetrating Peptides by Support Vector Machines, PLOS Computational Biology, 2011, 7(7), pp. 1-12; and Holton et al., CPPpred: prediction of cell penetrating peptides, Bioinformatics, 2013, 29(23), pp. 3094-3096, both herein incorporated by reference) and a CPP database is available (Gautam et al., CPPSite: a curated database of cell penetrating peptides, Database, 2012, Article ID bas015 and http://crdd.osdd.net/raghava/cppsite/index.php, both herein incorporated by reference). Accordingly, any suitable CPP may find utility in the invention and, as discussed below, a variety of CPPs have already been identified and tested and could form the basis for determining and identifying new CPPs.

CPPs may be derived from naturally-occurring proteins which are able to translocate across cell membranes such as the Drosophila homeobox protein Antennapedia (a transcription factor), viral proteins such as the HIV-1 transcriptional factor TAT and the capsid protein VP22 from HSV-1, and/or they may be synthetically-derived, e.g. from chimeric proteins or synthetic polypeptides such as polyarginine. As noted above, there is not a single mechanism responsible for the transduction effect and hence the design of CPPs may be based on different structures and sequences. Cell penetrating peptides are also reviewed in Jarver et al. 2006 (Biochimica et Biophysica Acta 1758, pages 260-263) and Table 1 below lists various representative peptides. U.S. Pat. No. 6,645,501 (herein incorporated by reference) further describes various cell penetrating peptides which might be used.

TABLE 1

| CPP | SEQUENCE | REFERENCE |
|---|---|---|
| Antp Class | | |
| Penetratin | RQIKIWFQNRRMKWKK | Bolton (2000) Eur. |
| | (SEQ ID NO: 8) | J. Neuro. 12:287 |
| Penetratin | RRMKWKK (SEQ ID NO: 21) | US 6472507 |
| derivatives | NRRMKWKK (SEQ ID NO: 22) | EP4855781 |
| | QNRRMKWKK (SEQ ID NO: 23) | WO 97/12912 |
| | FQNRRMKWKK (SEQ ID NO: 24) | |
| | RREKWKK (SEQ ID NO: 25) | |
| | RRQKWKK (SEQ ID NO: 26) | |
| | KRMKWKK (SEQ ID NO: 27) | |
| | RKMKWKK (SEQ ID NO: 28) | |
| | RROKWKK (SEQ ID NO: 29) | |
| | RRMKQKK (SEQ ID NO: 30) | |
| | RRMKWFK (SEQ ID NO: 31) | |
| | RORKWKK (SEQ ID NO: 32) | |
| | RRMWKKK (SEQ ID NO: 33) | |
| | RRMKKWK (SEQ ID NO: 34) | |
| | (O = ornithine) | |
| Protegrin Class | | |
| Pegelin (SynB) | RGGRLSYSRRRFSTSTGR | Rouselle, C. etal. |
| | (SEQ ID NO: 35) | (2000) Mol. |
| | | Pharm |
| | | 57:679 |
| HIV-TAT Class | | |
| HIV-TAT | GRKKRRQRRRPPQ | Vives E. J Biol, |
| | (SEQ ID NO: 36) | Chem 1997, |
| | | 272:16010 |
| | | Snyder (2004) |
| | | PLOS2: 186 |
| 47-57 OF | YGRKKRRQRRR | Potocky et al. |
| HIV-TAT | (SEQ ID NO: 37) | (2003) JBC |
| VP22 | DAATATRGRSAASRPTE | Elliott g. |
| | RPRAPARSASRPRRVD | Cell 1997, |
| | (SEQ ID NO: 38) | 88:223-233 |
| Amphipathic | | |
| peptides | | |
| MAP (also | KLALKLALKALKAALKLA | Morris MC., Nat |
| referred to | (SEQ ID NO: 9) | Biotechnol. 2001, |
| as KLA) | | 19:1173-1176 |
| Transportan | GWTLNSAGYLLGKINLKALAALAKKIL | Pooga M, FASEB |
| | (SEQ ID NO: 39) | J 1998, 12:67-77 |
| Transportan- | AGYLLGKINLKALAALAKKIL | Soomets U, |
| 10 | (SEQ ID NO: 40) | Biochim Biophys |
| | | Acta 2000, |
| | | 1467:165-176 |
| KALA | WEAKLAKALAKALAKHLAKALAKALKACEA | Oehike J., Biochim |
| | (SEQ ID NO: 41) | Biophys Acta |
| | | 1998, 1414:127- |
| | | 139 |
| MPG | GALFLGFLGAAGSTMGAWSQPKSKRKV | Wagstaff KM Curr |
| | (SEQ ID NO: 42) | Med Chem 2006, |
| | | 13:1371-1387 |
| Vectocell | VKRGLKLRHVRPRVTRMDV (SEQ ID NO: 43) | Coupade (2005) |
| peptides | SRRARRSPRHLGSG (SEQ ID NO: 44) | Biochem. J. 407 |
| | LRRERQSRLRRERQSR (SEQ ID NO: 45) | |
| | GAYDLRRRERQSRLRRRERQSR | |
| | (SEQ ID NO: 46) | |
| Other peptides | | |
| R5 | RRRRR (SEQ ID NO: 95) | Rothbard et al., |
| R6 | RRRRRR (SEQ ID NO: 96) | Nat. Med 6 (2000) |
| R7 | RRRRRRR (SEQ ID NO: 47) | 1253-1257 |
| R8 | RRRRRRRR (SEQ ID NO: 97) | |
| R9 | RRRRRRRRR (SEQ ID NO: 7) | |
| R10 | RRRRRRRRRR (SEQ ID NO: 98) | |
| R11 | RRRRRRRRRRR (SEQ ID NO: 48) | |

Antennapedia-derived CPPs (Antp class) represent a class of particular interest, based around the 16 amino acid Penetratin sequence as shown in Table 1, which corresponds to the third loop of antennapedia protein and was shown to be responsible for translocation of the protein. Penetratin has been extensively developed as a delivery vehicle, including particularly for pharmaceutical use, and a wide range of Penetratin derivatives and modified sequences have been proposed and described. Reference may be made in particular to WO 91/1891, WO 00/1417, WO 00/29427, WO 2004/069279 and U.S. Pat. No. 6,080,724 (herein incorporated by reference). The 16 amino acid sequence of Penetratin may be modified and/or truncated, or the peptide may be chemically-modified or retro-, inverso- or retro-inverso analogues may be made whilst retaining cell-penetrating activity. In a preferred embodiment the second peptide, i.e. the CPP, is penetratin. In this embodiment the CPP comprises, or preferably consists of, SEQ ID NO: 8. The CPP may alternatively be a variant of penetratin, i.e. it may comprise or consist of an amino acid sequence having at least 80%, 85% or 90% sequence identity to SEQ ID NO: 8. If a variant of penetratin is used, it is a functional variant, having cell penetration activity. Such activity may be tested as described above and below. Penetratin derivatives (e.g. peptides of SEQ ID NOs: 21-34) may alternatively be used as the CPP.

Another group of cell penetrating peptides which may advantageously be used are based on the HIV-TAT sequence and HIV-TAT and fragments thereof represent a preferred class of CPPs for use according to the present invention. Various TAT-based CPPs are described in U.S. Pat. No. 5,656,122 (herein incorporated by reference).

As mentioned above no particular structural features or sequence motifs are common to all CPPs. However, various classes of CPPs may be identified by particular features, such as for example peptides which are amphipathic and net positively charged. Other groups of CPPs may have a structure exhibiting high α-helical content. An amphipathic CPP of particular interest is the Model Amphipathic Peptide (MAP), also referred to herein as the KLA CPP. KLA has the amino acid sequence set forth in SEQ ID NO: 9. Thus in a particular preferred embodiment, the CPP comprises, or preferably consists of, the amino acid sequence set forth in SEQ ID NO: 9. The CPP may alternatively be a variant of KLA, i.e. it may comprise or consist of an amino acid sequence having at least 80%, 85% or 90% sequence identity to SEQ ID NO: 9. If a variant of KLA is used, it is a functional variant, having cell penetration activity. Such activity may be tested as described above and below.

Proline-rich amphipathic peptides are another class of CPP and such peptides, characterised by the presence of pyrrolidine rings from prolines, are described in Pujals et al. 2008 Advanced Drug Delivery Reviews 60, pages 473-484 (herein incorporated by reference). Other amphipathic CPPs (e.g. those of SEQ ID NOs: 39-46, which include Transportan and Vectocell peptides) may also be used.

Another group may be peptides characterised by a high content of basic amino acids. CPPs may thus be or may comprise oligomers of basic amino acids such as arginine e.g. 5 to 20, 6 to 15 or 6 to 12 arginine residues. That is to say the CPP may be a polyarginine peptide. Particular examples include R₇ (SEQ ID NO: 47), R₉ (SEQ ID NO: 7) and R₁₁ (SEQ ID NO: 48). A particularly preferred polyarginine CPP is R9, and thus the CPP may in particular comprise or consist of the amino acid sequence set forth in SEQ ID NO: 7.

Other successfully developed CPPs include pVEC (Elmquist et al. 2003 Biol. Chem 384, pages 387-393; Holm et al. 2005 Febs Lett. 579, pages 5217-5222, all herein incorporated by reference) and calcitonin-derived peptides (Krauss et al. 2004 Bioorg. Med. Chem. Lett., 14, pages 51-54 herein incorporated by reference).

Commercially available CPPs include Chariot, based on the Pep-1 peptide (Active Motif, France), the Syn-B vectors based on the protegrin peptide PG-1 (Syntem, France), and Express-si Delivery based on the MPG peptide from Genospectra, USA.

In some embodiments the CPPs may be cyclic peptides, such as those disclosed in Oh et al., 2014, Mol. Pharmaceutics, vol. 11, pp. 3528-3536 (incorporated herein by reference). In particular, the CPPs may be amphiphilic cyclic CPPs, particularly containing tryptophan and arginine residues. In some embodiments the CPPs may be cyclic polyarginine peptides and may be modified by the addition of a fatty acyl moiety, e.g. octanoyl, dodecanoyl, hexadecanoyl, N-acetyl-L-tryptophanyl-12-aminododecanoyl etc.

In addition to publically available and reported CPPs, novel or derivative CPPs may be designed and synthesized based on known or reported criteria (e.g. known CPP sequences or features such as basic amino acid content, a-helical content etc. as discussed above). Additionally, randomly-designed or other peptides may be screened for CPP activity, for example by coupling or attaching such a peptide containing a reporter molecule, e.g. a detectable label or tag such as a fluorescent tag to the desired cargo (e.g. an oligopeptidic compound as described herein) and testing to see if the construct is translocated across the cell membrane, for example by adding these peptides to live cells followed by examination of cellular import e.g. using confocal microscopy.

Indeed, whilst it is generally the case that a CPP will penetrate or enter virtually any cell type, it may in some cases be observed that successful or efficient delivery may be dependent, or may vary depending, on the precise nature of the cargo (e.g. cargo peptide sequence) and/or the CPP used. It would be well within the routine skill of the person skilled in the art to determine optimum peptide sequences and combinations etc., and to test and/or modify cargo and/or CPP sequence or structure etc.

In a particular embodiment, the CPP used in the oligopeptidic compound of the invention is penetratin (SEQ ID NO: 8), and the first peptide is selected from any one of SEQ ID NOs: 1 to 6, 16 to 20 or 104. For instance, the oligopeptidic compound may comprise penetratin as CPP and a first peptide of SEQ ID NO: 1. In another embodiment, the CPP used is nona-arginine (SEQ ID NO: 7), and the first peptide is selected from any one of SEQ ID NOs: 1 to 6, 16 to 20 or 104. For instance, the oligopeptidic compound may comprise nona-arginine as CPP and a first peptide of SEQ ID NO: 1. In another embodiment the CPP used in the oligopeptidic compound of the invention is KLA (SEQ ID NO: 9), and the first peptide is selected from any one of SEQ ID NOs: 1 to 6, 16 to 20 or 104. For instance, the oligopeptidic compound may comprise KLA as CPP and a first peptide of SEQ ID NO: 1.

The first and second oligopeptidic components of the oligopeptidic compound are covalently joined to each other. The covalent joining may be achieved by any means known in the art. In a particular and preferred embodiment, the oligopeptidic compound is a single oligopeptide chain comprising both the first and second peptides. In this embodiment the first and second peptides are directly or indirectly joined to each other by peptide bonds. By “directly” joined by peptide bonds is meant that the C-terminal amino acid of the first peptide is joined by a peptide bond to the N-terminal amino acid of the second peptide, or that the C-terminal amino acid of the second peptide is joined by a peptide bonds to the N-terminal amino acid of the first peptide. In other words, there are no additional amino acid residues located between the first and second peptides. By “indirectly” joined by peptide bonds is meant that the first and second peptide are located in the same oligopeptide chain, but are not directly joined to each other. Rather, if the first and second peptides are indirectly joined, they are separated by one or more amino acids. The one or more amino acids separating the first and second peptides may constitute a linker sequence, providing some separation between the first and second peptides. Alternatively, an additional functional amino acid sequence may be present between the first and second peptides. Preferably, the first and second peptides are indirectly joined to each other by peptide bonds, and are separated by a peptide linker.

Any suitable peptide linker may be used to separate the first and second peptides within the oligopeptide of the invention. In a particular and preferred embodiment the linker is a single amino acid residue. A single glycine residue is particularly suitable for this purpose, though other amino acids may also be used, particularly those with small side chains e.g. an alanine or a serine residue. Most preferably, the linker is a glycine residue. Longer linkers may also be used, such as a (G₄S)ₙ linker, comprising a series of repeats of the unit Gly-Gly-Gly-Gly-Ser. Examples include G₄S (a single G₄S unit), (G₄S)₂, (G₄S)₃, etc. Any other suitable linking peptide sequence may alternatively be used.

When the first and second oligopeptidic components are both located within a single oligopeptidic chain, the first and second oligopeptidic components can be arranged in either order. That is to say, the first component (e.g. peptide) may be N-terminal to the second component (e.g. peptide), or the second component (e.g. peptide) may be N-terminal to the first component (e.g. peptide). As shown in the Examples though, it is generally preferred that the first component (e.g. peptide) is located N-terminal to the second component (e.g. peptide), i.e. that the CPP is located C-terminal to the first component (e.g. peptide).

In a particularly preferred embodiment, the oligopeptidic compound of the invention is an oligopeptide consisting of the first peptide and the second peptide, separated by a linker. In this embodiment the oligopeptide does not comprise any additional sequence units, and the first and second peptide form the N- and C-terminus of the oligopeptide. As noted above, it is preferred that the first peptide is located at the N-terminus of the oligopeptide and the second peptide is located at the C-terminus of the oligopeptide. The linker may be any linker, as described above. In another embodiment, the oligopeptidic compound of the invention is an oligopeptide consisting of the first and second peptide (i.e. without a linker).

In another embodiment, the oligopeptidic compound of the invention is an oligopeptide comprising the first peptide and the second peptide. The oligopeptide of this embodiment may further comprise a linker. The oligopeptide may further comprise one or more additional amino acid sequence units, which may have structural or functional activity. Such additional amino acid sequence units may be located at the N-terminus or C-terminus of the oligopeptide, or between the first and second peptides.

Particularly preferred oligopeptides of the invention are set forth in SEQ ID NOs: 10-15, 107-113, 115 and 117. SEQ ID NO: 10 is also referred to as Pep7-Pen, and comprises Pep7 (SEQ ID NO: 1) at the N-terminus and penetratin (SEQ ID NO: 8) at the C-terminus, separated by a linker of a single glycine residue. SEQ ID NO: 11 is also referred to as Pep6-Pen, and comprises Pep6 (SEQ ID NO: 2) at the N-terminus and penetratin (SEQ ID NO: 8) at the C-terminus, separated by a linker of a single glycine residue. SEQ ID NO: 12 is also referred to as Pep7-KLA, and comprises Pep7 (SEQ ID NO: 1) at the N-terminus and KLA (SEQ ID NO: 9) at the C-terminus, separated by a linker of a single glycine residue. SEQ ID NO: 13 is also referred to as Pep7-Arg9, and comprises Pep7 (SEQ ID NO: 1) at the N-terminus and nona-arginine (SEQ ID NO: 7) at the C-terminus, separated by a linker of a single glycine residue. SEQ ID NO: 14 is also referred to as Pep7 (Y4A)-Pen, or P7-A4-Pen, and comprises Pep7(Y4A) (SEQ ID NO: 3) at the N-terminus and penetratin (SEQ ID NO: 8) at the C-terminus, separated by a linker of a single glycine residue. SEQ ID NO: 15 is also referred to as Pep7(T2N)-Pen, or P7-N2-Pen, and comprises Pep7(T2N) (SEQ ID NO: 4) at the N-terminus and penetratin (SEQ ID NO: 8) at the C-terminus, separated by a linker of a single glycine residue. SEQ ID NO: 107 is also referred to as Pep7(T2K)-Pen, or P7-K2-Pen, and comprises Pep7(T2K) (SEQ ID NO: 118) at the N-terminus and penetratin (SEQ ID NO: 8) at the C-terminus, separated by a linker of a single glycine residue. SEQ ID NO: 108 is also referred to as Pep7(T2H)-Pen, or P7-H2-Pen, and comprises Pep7(T2H) (SEQ ID NO: 18) at the N-terminus and penetratin (SEQ ID NO: 8) at the C-terminus, separated by a linker of a single glycine residue. SEQ ID NO: 109 is also referred to as Pep7(T2D)-Pen, or P7-D2-Pen, and comprises Pep7(T2D) (SEQ ID NO: 17) at the N-terminus and penetratin (SEQ ID NO: 8) at the C-terminus, separated by a linker of a single glycine residue. SEQ ID NO: 110 is also referred to as Pep7(Y4F)-Pen, or P7-F4-Pen, and comprises Pep7(Y4F) (SEQ ID NO: 19) at the N-terminus and penetratin (SEQ ID NO: 8) at the C-terminus, separated by a linker of a single glycine residue. SEQ ID NO: 111 is also referred to as Pep7(Y4N)-Pen, or P7-N4-Pen, and comprises Pep7(Y4N) (SEQ ID NO: 119) at the N-terminus and penetratin (SEQ ID NO: 8) at the C-terminus, separated by a linker of a single glycine residue. SEQ ID NO: 112 is also referred to as Pep7(Y4D)-Pen, or P7-D4-Pen, and comprises Pep7(Y4D) (SEQ ID NO: 120) at the N-terminus and penetratin (SEQ ID NO: 8) at the C-terminus, separated by a linker of a single glycine residue. SEQ ID NO: 113 is also referred to as Pep7(Y4S)-Pen, or P7-S4-Pen, and comprises Pep7(Y4S) (SEQ ID NO: 121) at the N-terminus and penetratin (SEQ ID NO: 8) at the C-terminus, separated by a linker of a single glycine residue. SEQ ID NO: 115 is also referred to as Pep7(T10N)-Pen, or P7-N10-Pen, and comprises Pep7(T10N) (SEQ ID NO: 104) at the N-terminus and penetratin (SEQ ID NO: 8) at the C-terminus, separated by a linker of a single glycine residue. SEQ ID NO: 117 is also referred to as Pep7(T10R)-Pen, or P7-R10-Pen, and comprises Pep7(T10R) (SEQ ID NO: 20) at the N-terminus and penetratin (SEQ ID NO: 8) at the C-terminus, separated by a linker of a single glycine residue.

In a particular embodiment, the oligopeptidic compound of the invention comprises or consists of the amino acid sequence set forth in SEQ ID NO: 10. In another embodiment, the oligopeptidic compound of the invention comprises or consists of the amino acid sequence set forth in SEQ ID NO: 11. In another embodiment, the oligopeptidic compound of the invention comprises or consists of the amino acid sequence set forth in SEQ ID NO: 12. In another embodiment, the oligopeptidic compound of the invention comprises or consists of the amino acid sequence set forth in SEQ ID NO: 13. In another embodiment, the oligopeptidic compound of the invention comprises or consists of the amino acid sequence set forth in SEQ ID NO: 14. In another embodiment, the oligopeptidic compound of the invention comprises or consists of the amino acid sequence set forth in SEQ ID NO: 15. In another embodiment, the oligopeptidic compound of the invention comprises or consists of the amino acid sequence set forth in SEQ ID NO: 107. In another embodiment, the oligopeptidic compound of the invention comprises or consists of the amino acid sequence set forth in SEQ ID NO: 108. In another embodiment, the oligopeptidic compound of the invention comprises or consists of the amino acid sequence set forth in SEQ ID NO: 109. In another embodiment, the oligopeptidic compound of the invention comprises or consists of the amino acid sequence set forth in SEQ ID NO: 110. In another embodiment, the oligopeptidic compound of the invention comprises or consists of the amino acid sequence set forth in SEQ ID NO: 111. In another embodiment, the oligopeptidic compound of the invention comprises or consists of the amino acid sequence set forth in SEQ ID NO: 112. In another embodiment, the oligopeptidic compound of the invention comprises or consists of the amino acid sequence set forth in SEQ ID NO: 113. In another embodiment, the oligopeptidic compound of the invention comprises or consists of the amino acid sequence set forth in SEQ ID NO: 115. In another embodiment, the oligopeptidic compound of the invention comprises or consists of the amino acid sequence set forth in SEQ ID NO: 117.

Particular oligopeptidic compounds of the invention comprising SEQ ID NO: 10 include SEQ ID NO: 10 itself, SEQ ID NO: 65, SEQ ID NO: 66 and SEQ ID NO: 105. SEQ ID NO: 65 is referred to in the Examples as P3sh1-Pen, and has the peptide P3sh1 (SEQ ID NO: 5) as the first oligopeptidic component; SEQ ID NO: 66 is referred to in the Examples as P3sh2-Pen, and has the peptide P3sh2 (SEQ ID NO: 6) as the first oligopeptidic component; and SEQ ID NO: 105 is referred to in the Examples as P3sh3-Pen, and has the peptide P3sh3 (SEQ ID NO: 16) as the first oligopeptidic component.

The oligopeptidic compound of the invention may alternatively comprise at least two separate peptides (one comprising the first peptide and the other comprising the CPP) joined by a non-peptide bond. Any suitable non-peptide covalent means of attachment can be used for this purpose. The peptides may be joined directly to each other, or indirectly, via a linker. The peptides may be joined to each other via amino acid side chains. For instance, if the peptides comprising each of the first and second peptides both contain a cysteine residue, the peptides may be joined by a disulphide bond. The peptides may alternatively be joined by an isopeptide bond, or any other suitable type of conjugation.

The oligopeptidic compounds of the invention may be synthesised by any method known in the art. Oligopeptides of the invention as described above, in which the first and second peptides are located in a single peptide chain, may be synthesised using a protein expression system. In this method of synthesis, a DNA sequence encoding the oligopeptidic compound may be cloned and introduced into an expression vector. Such a nucleotide sequence may be generated and synthesised by the skilled person without difficulty. As detailed further below, a nucleic acid encoding an oligopeptidic compound of the invention constitutes a further aspect of the invention.

A DNA sequence encoding the oligopeptidic compound described herein may be generated by amplification from a template, e.g. by PCR, or by artificial gene synthesis, using standard methods known in the art. The DNA sequence encoding the oligopeptidic compound may then be introduced into an expression vector, using standard molecular cloning techniques such as restriction enzymes or Gibson assembly. Suitable expression vectors are known in the art. The expression vector may then be introduced into a cellular expression system using standard techniques. Suitable expression systems may include bacterial cells and/or eukaryotic cells such as yeast cells, insect cells or mammalian cells.

Instead of a cellular expression system, a cell-free, in vitro protein expression system may be used to synthesise an oligopeptide according to the invention. In such a system a nucleotide sequence encoding the oligopeptidic compound is transcribed into mRNA, and the mRNA translated into a protein, in vitro. Cell-free expression system kits are widely commercially available, and can be purchased from e.g. Thermo Fisher Scientific (USA).

Oligopeptidic compounds according to the invention may alternatively be chemically synthesised in a non-biological system. Oligopeptidic compounds which comprise D-amino acids or other non-proteinogenic amino acids, or in which the first and second peptides are joined otherwise than via peptide bonds in the context of a single oligopeptide, may in particular be chemically synthesised, since biological synthesis is generally not possible in this case. Liquid-phase protein synthesis or solid-phase protein synthesis may be used to generate polypeptides which may form or be comprised within the oligopeptidic compounds of the invention. Such methods are well-known to the skilled person, who can readily produce oligopeptidic compounds using appropriate methodology common in the art.

In a second aspect, the invention provides a pharmaceutical composition comprising an oligopeptidic compound of the invention, and one or more pharmaceutically-acceptable diluents, carriers or excipients.

Pharmaceutically acceptable diluents, carriers and excipients are well known in the art, and the compositions of the invention may be formulated in any convenient manner according to techniques and procedures known in the pharmaceutical art. “Pharmaceutically acceptable” as used herein refers to ingredients that are compatible with other ingredients of the compositions as well as being physiologically acceptable to the recipient. The nature of the composition and carriers or excipient materials may be selected in routine manner according to choice and the desired route of administration, purpose of treatment etc. The pharmaceutical composition may be prepared for administration to a subject by any suitable means. Such administration may in particular be oral, topical, nasal or parenteral. Preferably, the composition is prepared for parenteral administration, which includes subcutaneous, intramuscular, intravenous, intraarterial, intraperitoneal and intradermal administration.

Pharmaceutical compositions as disclosed herein include in particular liquid solutions or syrups, though the composition may alternatively be provided in the form of a powder or as granules or suchlike, which may be dissolved in liquid to yield a solution.

Liquid pharmaceutical compositions, whether they be solutions, suspensions or other like form, may include one or more of the following: sterile diluents such as water for injection, saline solution, preferably physiological, Ringer's solution, isotonic sodium chloride, fixed oils such as synthetic mono- or diglycerides which may serve as a solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose. A parenteral preparation can be enclosed in ampoules, disposable syringes or multiple dose vials made of glass or plastic. An injectable pharmaceutical composition is preferably sterile.

Suitable excipients may include lactose, maize starch or derivatives thereof, stearic acid or salts thereof, vegetable oils, waxes, fats and polyols. Suitable carriers or diluents include carboxymethylcellulose (CMC), methylcellulose, hydroxypropylmethylcellulose (HPMC), dextrose, trehalose, liposomes, polyvinyl alcohol, pharmaceutical grade starch, mannitol, lactose, magnesium stearate, sodium saccharin, talcum, cellulose, glucose, sucrose (and other sugars), magnesium carbonate, gelatin, oil, alcohol, detergents and emulsifiers such as polysorbates. Stabilising agents, wetting agents, emulsifiers, sweeteners etc. may also be used.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials. The skilled clinician will be able to calculate an appropriate dose for a patient based on all relevant factors, e.g. age, height, weight, the condition to be treated and its severity.

In a third aspect the invention provides an oligopeptidic compound of the invention or pharmaceutical composition of the invention for use in therapy. By “therapy” as used herein is meant the treatment or prevention of any disease or pathological condition. Treatment may be intended to cure the disease or condition (i.e. it may be or intended to be curative treatment), or it may be palliative (i.e. treatment designed merely to limit, relieve or improve the symptoms of a condition). Treatment may be life-extending even if non-curative. When used in therapy, the oligopeptidic compound or pharmaceutical composition according to the present invention may be administered to a subject by any suitable means. For instance, the oligopeptidic compound or pharmaceutical composition may be administered to a subject by oral, topical, nasal or parenteral means. Preferably, the oligopeptidic compound or pharmaceutical composition is administered to the subject by parenteral means, which includes subcutaneous, intramuscular, intravenous, intraarterial, intraperitoneal and intradermal administration.

In particular, provided herein is an oligopeptidic compound of the invention or pharmaceutical composition of the invention for use in prevention or treatment of a disease associated with hypercytokinemia in a human subject. The oligopeptidic compound of the invention used for prevention or treatment of a disease associated with hypercytokinemia is generally an oligopeptidic compound comprising a first oligopeptidic component which is capable of blocking signalling from TLR4, TLR7, TLR8 and/or TLR9, as described above. For instance, the first oligopeptidic component may bind TRAM and inhibit the interaction between TRAM and SLAMF1, and/or inhibit the formation of the Myddosome complex (comprising Mal, MyD88, IRAK1 and IRAK4). As well as blocking signalling from TLR4, TLR7, TLR8 and/or TLR9, the first oligopeptidic component of the compound may have an anti-proliferative or cytotoxic effect on cancer cells, though this is not required and is strictly optional. The pharmaceutical composition of the invention used for prevention or treatment of a disease associated with hypercytokinemia generally comprises such an oligopeptidic compound.

In a preferred embodiment of the invention, the first oligopeptidic component of the oligopeptidic compound used for prevention or treatment of a disease associated with hypercytokinemia comprises or consists of the amino acid sequence set forth in SEQ ID NO: 1 (P7), SEQ ID NO: 2 (P6), SEQ ID NO: 3 (P7-A4), SEQ ID NO: 4 (P7-N2), SEQ ID NO: 5 (P3sh1), SEQ ID NO: 6 (P3sh2), SEQ ID NO: 16 (P3sh3), SEQ ID NO: 17 (P7-D2), SEQ ID NO: 18 (P7-H2), SEQ ID NO: 19 (P7-F4), SEQ ID NO: 20 (P7-R10) or SEQ ID NO: 104 (P7-N10), or an amino acid sequence having at least 70% amino acid sequence identity to any aforesaid sequence, more particularly at least 75, 80, 85, 90, 95, 96, 97, 98 or 99% sequence identity.

In another preferred embodiment of the invention, the oligopeptidic compound used for prevention or treatment of a disease associated with hypercytokinemia comprises or consists of the amino acid sequence set forth in SEQ ID NO: 10 (P7-Pen), SEQ ID NO: 11 (P6-Pen), SEQ ID NO: 12 (P7-KLA), SEQ ID NO: 13 (P7-Arg9), SEQ ID NO: 14 (P7-A4-Pen), SEQ ID NO: 15 (P7-N2-Pen), SEQ ID NO: 108 (P7-H2-Pen), SEQ ID NO: 109 (P7-D2-Pen), SEQ ID NO: 110 (P7-F4-Pen), SEQ ID NO: 115 (P7-N10-Pen) or SEQ ID NO: 117 (P7-R10-Pen), or an amino acid sequence having at least 70% amino acid sequence identity to any aforesaid sequence, more particularly at least 75, 80, 85, 90, 95, 96, 97, 98 or 99% sequence identity.

Where the amino acid sequence is a variant amino acid sequence falling within the % sequence identity constraints set out above and where a particular amino acid substitution is specified, the specified amino acid substitution of the amino acid sequence in question is retained (e.g. the A4, N2, D2 substitution etc.).

The subject treated according to this aspect of the invention may be any human, male or female, of any age or size, who is suffering from or at risk of developing a hypercytokinemia-associated disease. Such a subject may be identified by a clinician.

Hypercytokinemia (also known as a cytokine storm or cytokine cascade) is a disease or condition resulting from a potentially fatal immune reaction consisting of a positive feedback loop between cytokines and white blood cells (e.g. monocytes or macrophages) with highly elevated (hyperexcessive) levels of various cytokines. Hypercytokinemia may be viewed as the systemic expression of a healthy and vigorous immune system resulting in the release of various inflammatory mediators, particularly cytokines. Both pro-inflammatory cytokines and anti-inflammatory cytokines are elevated in the serum of patients with hypercytokinemia and, as discussed below, hypercytokinemia may result from a number of infectious and non-infectious diseases. Hypercytokinemia includes any setting or condition in which excess cytokine production drives dangerous hyperinflammation. The term “cytokine” as used herein includes chemokines, and hypercytokinemia may include the overexpression of any cytokine, including chemokine, involved in the excessive immune response.

The cytokines which are overexpressed in hypercytokinemia can include Type I interferons, including IFNα and IFNβ. As discussed above and shown below, the inventors have shown that application of the oligopeptidic compounds of the invention to immune cells results in downregulation of expression of pro-inflammatory cytokines including IFNβ, CXCL-10, TNF, IL-1β and IL-6. The oligopeptidic compounds of the invention can thus inhibit or at least reduce the production of a number of pro-inflammatory cytokines and can thus prevent or treat hypercytokinemia. By prevent or treat as used herein does not mean that the therapy absolutely prevents or fully treats the hypercytokinemia-associated disease (though in a preferred embodiment this may be the case), merely that it results in an improvement in the condition of the subject relative to that which is seen without the therapy. Thus therapy with the binding agent of the present invention may merely improve the condition of the subject or relieve or partially relieve the symptoms of the disease, or result in a less severe condition occurring than might have occurred otherwise.

The hypercytokinemia-associated disease to be treated in the present invention may be characterised by up-regulation of expression of type I interferons (such as IFNβ), IL-13, IL-6 and/or TNF. In other words, in the hypercytokinemia-associated disease to be treated one or more of these cytokines is expressed at an elevated level relative to that of a healthy person, or more pertinently at an elevated level relative to a person who is suffering from an infection but whose immune system has responded in a controlled manner. In particular, type I interferons (e.g. IFNβ), IL-1β, IL-6 and/or TNF may be expressed at a level which is considered to be above a normal or healthy range. A skilled person will be able to identify whether the expression of any one of these cytokines is up-regulated in a particular subject using standard methods in the art, e.g. analysis of a blood sample by standard clinical biochemistry. The levels of these cytokines, may be analysed by standard techniques, e.g. ELISA or suchlike, and kits for such analysis are commercially available.

In a particularly preferred embodiment of the invention, the hypercytokinemia-associated disease is sepsis. Sepsis is a life-threatening condition in which an infection, most commonly a bacterial infection, induces an immune response which goes awry, leading to an uncontrolled immune response and hypercytokinemia. If untreated, sepsis is fatal, due to the damage caused by hypercytokinemia to the subject's organs which leads to multiple organ dysfunction syndrome (i.e. multiple organ failure). Sepsis is challenging to diagnose and treat, and as discussed above new treatments are urgently needed.

The symptoms and complications associated with sepsis are thought to arise from the overproduction of pro-inflammatory cytokines in blood. The introduction or administration of the agents of the invention, e.g. intravenously, can therefore be expected to treat or prevent sepsis by reducing or inhibiting the release of said cytokines in blood, in particular by reducing or inhibiting the release of IFNβ, IL-1β, IL-6 and TNF. The binding agents of the invention may thus be used to treat or prevent sepsis.

The infectious agent responsible for inducing sepsis is most commonly a bacterium (though a parasite, fungus or virus may be responsible). Theoretically, any pathogenic bacterial species may induce sepsis, and both Gram-negative and Gram-positive bacteria are commonly responsible. The hypercytokinemia-associated disease to be treated by the invention may thus be associated with a bacterial infection, particularly when the disease to be treated is sepsis. Severe viral infections are also commonly associated with hypercytokinemia. Viruses which cause acute respiratory distress syndrome (ARDS) commonly induce hypercytokinemia, including SARS-CoV-2, the causative agent of COVID-19, MERS-CoV and highly pathogenic influenza. Hypercytokinemia is also associated with the development of haemorrhagic fevers caused by viruses such as the Ebola, Marburg, Lassa, Crimean-Congo haemorrhagic fever and Dengue fever (DF) viruses. Thus the hypercytokinemia-associated disease to be treated by the invention may be associated with a viral infection.

In particular, as detailed above and in the examples below, the oligopeptidic compounds of the invention inhibit signalling through TRAM-SLAMF1 blocking TLR4-mediated expression of cytokines including IFNβ, TNF and CXCL-10. The compounds also inhibit signalling through the Myddosome, blocking TLR4-mediated expression of cytokines including IL-18 and IL-6. As noted above, TLR4 primarily recognises lipopolysaccharide (LPS). LPS are large molecules found in the outer membrane of Gram-negative bacteria. Thus in a preferred embodiment of the invention, the oligopeptidic compound according to the invention is for use in the treatment or prevention of a hypercytokinemia-associated disease which is associated with infection by a Gram-negative bacterium, in particular sepsis induced by infection by a Gram-negative bacterium.

The Gram-negative bacterium may be any Gram-negative bacterium, e.g. Pseudomonas aeruginosa, Vibrio cholerae, Actinobacillus pleuropneumoniae, Acinetobacter baumanii, Pasteurella haemolytica, Pasteurella multocida, Legionella pneumophila, Salmonella enterica (including Salmonella Typhi and Salmonella Typhimurium), Klebsiella pneumoniae, Klebsiella oxytoca, Brucella abortus, Chlamydia trachomatis, Chlamydia psittaci, Coxiella bumetti, Escherichia coli, Neiserria meningitidis, Neiserria gonorrhea, Haemophilus influenzae, Haemophilus ducreyi, Yersinia pestis, Yersinia enterolitica, Burkholderia cepacia, Burkholderia pseudomallei, Francisella tularensis, Bacteroides fragilis, Fusobacterium nucleatum, Proteus vulgaris, Cowdria ruminantium, Campylobacter jejuni, Helicobacter pylori, Shigella flexneri, Shigella dysenteriae, and Shigella sonnei.

In particular, the hypercytokinemia-associated disease to be treated or prevented using the binding agent of the current invention may be associated with infection by E. coli, P. aeruginosa, K. pneumoniae and/or K. oxytoca. The disease may be associated with infection by one or more Gram-negative bacterium, or indeed one or more Gram-negative bacterium and one or more other pathogen. In particular, the disease to be treated or prevented may be sepsis associated with infection by E. coli, P. aeruginosa, K. pneumoniae and/or K. oxytoca.

As detailed above, many other TLRs signal through the Myddosome when activated and are inhibited by the peptides of the invention (particularly those set out as preferred for use in the treatment of a disease associated with hypercytokinemia), including TLRs 8 and 9, which recognise bacterial RNA and DNA, particularly from Gram-positive bacteria. Furthermore, TLR4 is also capable of recognising LTA from some Gram-positive bacterial species. Thus in another embodiment of the invention, the oligopeptidic compound according to the invention is for use in the treatment or prevention of a hypercytokinemia-associated disease which is associated with infection by a Gram-positive bacterium, in particular sepsis induced by infection by a Gram-positive bacterium.

The Gram-positive bacterium may be any Gram-positive bacterium, e.g. Staphylococcus aureus (including methicillin-resistant Staphylococcus aureus (MRSA) and vancomycin-resistant Staphylococcus aureus (VRSA)), Streptococcus pyogenes, Streptococcus agalactiae, Streptococcus anginosus, Streptococcus intermedius, Streptococcus constellatus, Streptococcus dysgalactiae, Streptococcus dysgalactiae subsp. equisimilis, Streptococcus pneumoniae, Group A Streptococci species, Group B Streptococci species, Group C Streptococci species, Group D Streptococci species, Enterococcus faecalis or Enterococcus faecium (including vancomycin-resistant Enterococci (VRE)), Staphylococcus epidermidis and Staphylococcus haemolyticus.

In particular, the hypercytokinemia-associated disease to be treated or prevented using the oligopeptidic compound of the current invention may be associated with infection by S. aureus (including MRSA), S. pyogenes or an Enterococcus, and in particular may be sepsis associated with infection by these species. The disease may be associated with infection by one or more Gram-positive bacterium, or indeed one or more Gram-positive bacterium and one or more other pathogen. For instance, the disease (e.g. sepsis) may be associated with infection by one or more Gram-positive bacteria and one or more Gram-negative bacteria.

As also detailed above, TLR7, TLR8 and TLR9 (signalling from which is blocked by the peptides of the invention) all recognise viral nucleic acids. Thus in another embodiment of the invention, the oligopeptidic compound according to the invention (particularly an oligopeptidic compound of the invention as set out above as particularly suitable for use in the treatment of a disease associated with hypercytokinemia) is for use in the treatment or prevention of a hypercytokinemia-associated disease which is associated with infection by a virus. The hypercytokinemia-associated disease may be sepsis, though this is less common in viral infections. Respiratory viruses such as influenza (particularly highly pathogenic avian influenzas) and coronaviruses, such as MERS and SARS-CoV-2, are associated with hypercytokinemia resulting in ARDS. As detailed above, haemorrhagic fevers caused by viruses such as the Ebola, Marburg, Lassa, Crimean-Congo haemorrhagic fever and Dengue fever (DF) viruses are also associated with hypercytokinemia, which leads to disseminated intravascular coagulation and vascular dysfunction and collapse. In the cases of both respiratory and haemorrhagic viruses, the effects of hypercytokinemia can lead to multiple organ failure. Thus the hypercytokinemia-associated disease to be treated or prevented using the oligopeptidic compound of the invention may be associated with infection by a virus, particularly a respiratory virus, such as an influenza virus or a coronavirus, or a haemorrhagic virus. The hypercytokinemia-associated disease to be treated or prevented using the oligopeptidic compound of the invention may in particular be ARDS associated with infection by a respiratory virus (as detailed above) or a haemorrhagic fever associated with infection by a haemorrhagic virus (as detailed above).

The treatment or prevention of a hypercytokinemia-associated disease using the oligopeptidic compound of the present invention may comprise administering to the subject at least one second therapeutically active agent (i.e. administering at least one therapeutically active agent in addition to the oligopeptidic compound of the invention). Such a therapeutically active agent may be any agent which has a therapeutic effect, i.e. a preventative, curative or palliative effect on the disease to be treated. In particular embodiments, the second therapeutically active agent is an antibiotic. The term “antibiotic” as used herein is used in its common medical sense, i.e. a drug which kills or inhibits the growth of bacteria. An antibiotic may in particular be used in combination with the oligopeptidic compound of the invention to treat or prevent a hypercytokinemia-associated disease associated with a bacterial infection. However, an antibiotic may also be used in combination with the oligopeptidic compound of the invention to treat or prevent a hypercytokinemia-associated disease associated with a viral infection, in order to treat or prevent a secondary bacterial infection.

The oligopeptidic compound of the present invention may be administered to the subject in conjunction with one or more antibiotics. The antibiotic may in particular be a broad-spectrum antibiotic and/or a β-lactam.

In another embodiment, the second therapeutically active agent is an antiviral agent, i.e. an agent which inhibits viral replication or infection (e.g. viral cell entry). An antiviral agent may in particular be used in combination with the oligopeptidic compound of the invention to treat or prevent a hypercytokinemia-associated disease associated with a viral infection. Suitable antiviral agents may be selected based on the infecting virus. The second therapeutically active agent may alternatively be an anti-inflammatory agent, such as a non-steroidal anti-inflammatory drug (NSAID) or a corticosteroid. An anti-inflammatory agent may act in concert with the oligopeptidic compound of the invention to reduce the production of pro-inflammatory cytokines and thus prevent or treat a hypercytokinemia-associated disease (of any cause).

The oligopeptidic compound according to the present invention may be administered to a subject with the second therapeutically active agent, that is to say in the same solution as the second agent. Alternatively the binding agent may be administered to a subject separately to the second therapeutically active agent. When administered separately, the two or more drugs may be administered to the subject concurrently or consecutively.

In another aspect, the invention provides an oligopeptidic compound or pharmaceutical composition of the invention for use in the treatment or prevention of an inflammatory disease, an autoimmune disease, cancer or ischemia-reperfusion injury. An association between Myddosome-mediated signalling and these diseases/conditions has previously been reported (Singer et al., supra).

The oligopeptidic compound of the invention used for treatment or prevention of an inflammatory disease, an autoimmune disease or ischemia-reperfusion injury is generally an oligopeptidic compound comprising a first oligopeptidic component which is capable of blocking signalling from TLR4, TLR7, TLR8 and/or TLR9, as described above. As noted above, an association between Myddosome-mediated signalling and these diseases/conditions has previously been reported and thus the oligopeptidic component of the compound is generally capable of inhibiting the formation of the Myddosome complex. As well as blocking signalling from TLR4, TLR7, TLR8 and/or TLR9, the first oligopeptidic component of the compound may have an anti-proliferative or cytotoxic effect on cancer cells, though this is not required and is strictly optional. The pharmaceutical composition of the invention used for treatment or prevention of an inflammatory disease, an autoimmune disease or ischemia-reperfusion injury generally comprises such an oligopeptidic compound.

The first oligopeptidic component of the oligopeptidic compound used for treatment or prevention of an inflammatory disease, an autoimmune disease or ischemia-reperfusion injury, and the oligopeptidic compound itself, are generally the same as used for prevention or treatment of a disease associated with hypercytokinemia, as detailed above. That is to say, in a particular embodiment the first oligopeptidic component of the oligopeptidic compound used for treatment or prevention of an inflammatory disease, an autoimmune disease or ischemia-reperfusion injury comprises or consists of the amino acid sequence set forth in any one of SEQ ID NOs: 1-6, 16-20 or 104, or an amino acid sequence having at least 70% amino acid sequence identity to any aforesaid sequence, more particularly at least 75, 80, 85, 90, 95, 96, 97, 98 or 99% sequence identity, as defined above. In another particular embodiment the oligopeptidic compound used for treatment or prevention of an inflammatory disease, an autoimmune disease or ischemia-reperfusion injury comprises or consists of the amino acid sequence set forth in any one of SEQ ID NOs: 10-15, 108-110, 115 or 117, or an amino acid sequence having at least 70% amino acid sequence identity to any aforesaid sequence, more particularly at least 75, 80, 85, 90, 95, 96, 97, 98 or 99% sequence identity, as defined above.

The subject treated according to this aspect of the invention is preferably a human subject. The subject may have, or be at risk of developing, a disease or condition as listed above. Such a subject may be identified by a clinician.

In a particular embodiment, the oligopeptidic compound or pharmaceutical composition of the invention may be used to treat or prevent an inflammatory disease or autoimmune disease selected from inflammatory bowel disease (including Crohn's disease and ulcerative colitis), rheumatoid arthritis, systemic lupus erythematosus (SLE), Schnitzler's syndrome, atherosclerosis, graft-versus-host disease, cryopyrin-associated periodic syndromes and a fibrotic disease. Fibrotic diseases can affect any organ, including the heart, lung (pulmonary fibrosis), kidney, liver (cirrhosis) and bone marrow (myelofibrosis). Any such fibrotic disease may be treated or prevented according to the present invention. The oligopeptidic compound of the invention may also be used to treat or prevent scleroderma, a systemic fibrotic disease. Other inflammatory diseases which may be treated or prevented using the oligopeptidic compound or pharmaceutical composition of the invention include type II diabetes, neurodegenerative diseases (e.g. Alzheimer's disease or Parkinson's disease), gout and non-alcoholic steatohepatitis.

Further examples of inflammatory or autoimmune diseases which can be treated according to the present invention include multiple sclerosis (MS), ankylosing spondylitis, spondyloarthritis, psoriasis, psoriatic arthritis, osteoarthritis, degenerative arthritis, polymyalgia rheumatic, polymyositis, fibromyalgia, nephritis, Addison's disease, Grave's disease, autoimmune uveoretinitis, pemphigus vulgaris, autoimmune thyroiditis, uveitis, Behcet's disease and Sjögren's syndrome.

Another aspect of the invention provides an oligopeptidic compound of the invention, or a pharmaceutical composition of the invention, for use in treating cancer. The oligopeptidic compound of the invention used for treatment of cancer is generally an oligopeptidic compound comprising a first oligopeptidic component which has an anti-proliferative or cytotoxic effect on cancer cells, as described above. As well as having an anti-proliferative or cytotoxic effect on cancer cells, the first oligopeptidic component of the compound may be capable of blocking signalling from TLR4, TLR7, TLR8 and/or TLR9, though this is optional. The pharmaceutical composition of the invention for use in treating cancer generally comprises such an oligopeptidic compound.

In a preferred embodiment of the invention, the first oligopeptidic component of the oligopeptidic compound used for treating cancer comprises or consists of the amino acid sequence set forth in SEQ ID NO: 1 (P7), SEQ ID NO: 2 (P6), SEQ ID NO: 3 (P7-A4), SEQ ID NO: 118 (P7-K2), SEQ ID NO: 119 (P7-N4), SEQ ID NO: 120 (P7-D4) or SEQ ID NO: 121 (P7-S4), or an amino acid sequence having at least 70% amino acid sequence identity to any aforesaid sequence, more particularly at least 75, 80, 85, 90, 95, 96, 97, 98 or 99% sequence identity.

In another preferred embodiment of the invention, the oligopeptidic compound used for treating cancer comprises or consists of the amino acid sequence set forth in SEQ ID NO: 10 (P7-Pen), SEQ ID NO: 11 (P6-Pen), SEQ ID NO: 12 (P7-KLA), SEQ ID NO: 13 (P7-Arg9), SEQ ID NO: 14 (P7-A4-Pen), SEQ ID NO: 107 (P7-K2-Pen), SEQ ID NO: 111 (P7-N4-Pen), SEQ ID NO: 112 (P7-D4-Pen) or SEQ ID NO: 113 (P7-S4-Pen), or an amino acid sequence having at least 70% amino acid sequence identity to any aforesaid sequence, more particularly at least 75, 80, 85, 90, 95, 96, 97, 98 or 99% sequence identity.

As noted above, where the amino acid sequence is a variant amino acid sequence falling within the % sequence identity constraints set out above and where a particular amino acid substitution is specified, the specified amino acid substitution of the amino acid sequence in question is retained.

The oligopeptidic compound or pharmaceutical composition of the invention may be used to treat any cancer. The term “cancer” as used herein has its common meaning in the art, i.e. a malignant neoplasm. Any type of cancer may be treated according to the present invention, including both solid cancers (i.e. cancers comprising solid tumours) and haematological cancers. Cancers which may be treated according to the present invention include carcinoma (including adenocarcinoma, squamous cell carcinoma, basal cell carcinoma, transitional cell carcinoma, etc.), sarcoma, leukaemia and lymphoma. According to the invention, cancer of any stage (or grade) may be treated, including stage I, stage II, stage III and stage IV cancer. Both metastatic and localised (i.e. non-metastatic) cancer may be treated.

The oligopeptidic compound or pharmaceutical composition of the invention may in particular be used to treat or prevent a cancer selected from breast cancer, hepatocellular carcinoma, head and neck squamous cell carcinoma, lung cancer, melanoma, endometrial cancer, a haematological cancer (or blood cancer), cervical cancer, ovarian cancer, colorectal cancer or pancreatic cancer.

In one preferred embodiment, the oligopeptidic compound or pharmaceutical composition of the invention is used to treat colorectal cancer. In a particularly preferred embodiment the oligopeptidic compound or pharmaceutical composition of the invention is used to treat a haematological cancer, such as multiple myeloma, a leukemia or a lymphoma. Blood cancers including acute myeloid leukemia (AML), acute lymphoblastic leukemia (ALL) including particularly T cell ALL, mixed lineage leukemias (MLLs), T cell leukemia, activated B cell-like diffuse large B cell lymphoma (ABC-DLBCL), Hodgkin lymphoma, non-Hodgkin lymphoma, chronic lymphocytic leukemia (CLL), Waldenström macroglobulinemia (WM), follicular lymphoma, Myelodysplastic syndromes (MDSs), Myeloproliferative neoplasms (MPNs) and Kaposi sarcoma-associated herpesvirus (KSHV)-associated malignancies may be treated or prevented according to the present invention. Most preferably, the oligopeptidic compound or pharmaceutical composition of the invention is used to treat multiple myeloma.

In these embodiments of the invention (i.e. therapeutic uses of the oligopeptidic compounds and pharmaceutical compositions of the invention), the oligopeptidic compound may be administered to the subject in combination with a second therapeutically active agent. The second therapeutically active agent used is an agent useful in treating or preventing the relevant condition. Thus for instance, for the treatment or prevention of an inflammatory condition or autoimmune disease, the oligopeptidic compound may be administered in combination with an anti-inflammatory agent, as described above. For the treatment or prevention of cancer the oligopeptidic compound may be administered in combination with an anti-neoplastic agent, such as a chemotherapy agent, a radiotherapy agent, a hormonal therapy agent, a biologic agent and/or an immunotherapy agent, or a small molecule inhibitor. Such agents are well known in the art, and any such agent may be administered as a combined therapy with an oligopeptidic compound of the invention, as deemed suitable by a physician.

In a particular embodiment, an oligopeptidic compound of the invention is used for cancer treatment in combination with the chemotherapy agent melphalan. Melphalan has the structure set forth in formula I:

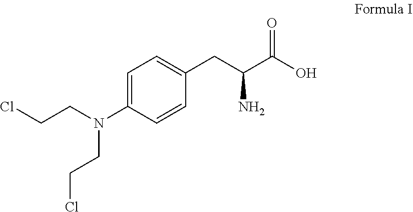 [SMILES: N[C@@H](Cc1ccc(N(CCCl)CCCl)cc1)C(=O)O]

Melphalan is an alkylating agent used for the treatment of multiple myeloma, ovarian cancer and melanoma. In particular embodiments, an oligopeptidic compound of the invention is used in combination with melphalan, or an alkylating agent more generally, in the treatment of multiple myeloma, ovarian cancer or melanoma. The Examples below demonstrate that the combination of a peptide of the invention and melphalan has a synergistic effect in the killing of multiple myeloma cells. In a preferred embodiment an oligopeptidic compound of the invention is used in combination with melphalan in the treatment of multiple myeloma.

The oligopeptidic compound of the invention may be used in therapy in combination with any other known drug for treatment of the same disease. For instance, in the case of multiple myeloma the oligopeptidic compound may be used in combination with a proteasome inhibitor, e.g. bortezomib, carfilzomib or ixazomib.

The embodiments of the invention discussed above may alternatively be seen to provide a method of treating or preventing a disease in a subject, comprising administering to the subject an oligopeptidic compound or pharmaceutical composition of the invention, wherein the disease is as defined above. That is to say, the method is for treating or preventing a disease selected from a hypercytokinemia-associated disease, an inflammatory disease, an autoimmune disease, cancer or ischemia-reperfusion injury. Examples of such conditions, which may be treated or prevented according to the present invention, are set out above. The particular oligopeptidic compound of the invention used to treat each condition is generally selected as detailed above. The subject treated according to the invention is preferably a human subject. Such a human subject is a subject in need of treatment or prevention of a disease or condition as listed above. A human subject in need of such treatment can be identified by a clinician.

In this method of treating or preventing a disease, all aspects of the treatment or prevention may be as described above.

When an oligopeptidic compound of the invention is administered to a human subject to treat or prevent a disease as described above, e.g. a hypercytokinemia-associated disease, a therapeutically effective amount of the compound should be administered. A therapeutically effective amount, or dose, of the compound is an amount sufficient to display a therapeutic effect (as defined above, a therapeutic effect may be any improvement in the condition of the subject, including a curative effect, relief or partial relief of the symptoms of the disease, or a reduction in the severity of the condition which occurs or would otherwise have occurred). A therapeutic amount may thus be medically calculated. According to the invention, a therapeutic effect of the binding agent may alternatively be a reduction in the level of pro-inflammatory cytokines in the blood of the subject, in particular a reduction in the level of IFNβ, CXCL-10, TNF, IL-1β and/or IL-6. Such levels may easily be measured by the skilled person. When the binding agent is administered in conjunction with at least one second therapeutically active agent, the second therapeutically active agent(s) are preferably also administered in a therapeutically effective amount.

These aspects of the invention may alternatively be seen to provide the use of an oligopeptidic compound as defined herein in the manufacture of a medicament for use in the treatment or prevention of a disease in a subject (preferably a human subject), wherein the disease is selected from a hypercytokinemia-associated disease, an inflammatory disease, an autoimmune disease, cancer or ischemia-reperfusion injury. Examples of such conditions, which may be treated or prevented according to the present invention, are set out above. In this aspect of the invention, all aspects of the disease treatment or prevention may be as described above.

In a further aspect, the invention provides a kit comprising an oligopeptidic compound or pharmaceutical composition of the invention, and a second therapeutically active agent. The second therapeutically active agent may in particular be an antibiotic, an antiviral or an anti-inflammatory agent, as described above. Alternatively the second therapeutically active agent may be an antineoplastic agent, as described above. The kit may comprise a first container comprising the oligopeptidic compound/pharmaceutical composition and a second container comprising the second therapeutically active agent. Alternatively, the kit may comprise a single container comprising both the oligopeptidic compound and the second therapeutically active agent, e.g. in a single pharmaceutical composition. The oligopeptidic compound and second therapeutically active agent may be provided in the kit in any suitable form, e.g. in solution. The kit may be used to treat or prevent a disease in a subject. Diseases which may be treated according to the present invention are described above. Preferably the subject is a human subject. Alternatively, the kit may be used for research, e.g. for use in an animal model of disease.

In a preferred embodiment, the kit comprises an oligopeptidic compound of the invention which is capable of blocking signalling from TLR4, TLR7, TLR8 and/or TLR9, and a second therapeutically active agent which is an antibiotic, an antiviral or an anti-inflammatory agent.

In another preferred embodiment, the kit comprises an oligopeptidic compound of the invention which has an anti-proliferative and/or cytotoxic effect on cancer cells, and a second therapeutically active agent which is an antineoplastic agent. The antineoplastic agent may in particular be a chemotherapy, radiotherapy, hormonal therapy and/or immunotherapy agent, and/or a small molecule inhibitor or biologic, as discussed above. In a particular embodiment the antineoplastic agent is suitable for treating a haematological cancer, particularly multiple myeloma. For instance, the antineoplastic agent may be a proteasome inhibitor (as detailed above) or an alkylating agent, e.g. melphalan.

The invention also provides a product comprising an oligopeptidic compound or pharmaceutical composition of the invention, and a second therapeutically active agent, as a combined preparation for separate, simultaneous or sequential use in the treatment or prevention of a disease in a subject. The disease to be treated or prevented according to this aspect of the invention is any disease as described above, that can be treated according to the present invention. The subject is preferably a human subject.

The second therapeutically active agent is preferably an antibiotic, an antiviral, an anti-inflammatory or an antineoplastic agent, as described above. The oligopeptidic compound and second therapeutically active agent may be provided within the product in any suitable manner, as described above in respect of the kit. Thus in the product the oligopeptidic compound/pharmaceutical composition may be provided in a first container and the second therapeutically active agent in a separate, second container. Accordingly, both the oligopeptidic compound and second therapeutically active agent may be provided in a single container, e.g. in the context of a pharmaceutical composition comprising both the oligopeptidic compound of the invention and the second therapeutically active agent.

In a particular embodiment, the product comprises an oligopeptidic compound of the invention which is capable of blocking signalling from TLR4, TLR7, TLR8 and/or TLR9, and the product is for use in the treatment or prevention of a disease associated with hypercytokinemia, an inflammatory disease, an autoimmune disease or ischemia-reperfusion injury. In this embodiment, the second therapeutically active agent may be selected from an antibiotic, an antiviral or an anti-inflammatory agent, as described above.

In another embodiment, product comprises an oligopeptidic compound of the invention which has an anti-proliferative and/or cytotoxic effect on cancer cells, and the product is for use in the treatment of cancer. In this embodiment, the second therapeutically active agent may be an antineoplastic agent, in particular a chemotherapy, radiotherapy, hormonal therapy and/or immunotherapy agent, and/or a small molecule inhibitor or biologic, as discussed above. In a particular embodiment the antineoplastic agent is suitable for treating a haematological cancer, particularly multiple myeloma. For instance, the antineoplastic agent may be a proteasome inhibitor (as detailed above) or an alkylating agent, e.g. melphalan.

In another aspect the invention provides a nucleic acid molecule which comprises a nucleotide sequence which encodes an oligopeptidic compound of the invention. The nucleic acid molecule of the invention may be an isolated nucleic acid molecule. As used herein, “isolated” means that the nucleic acid molecule is separated or purified from any natural environment in which it might be found. The nucleic acid molecule of the invention may include DNA or RNA or chemical derivatives of DNA or RNA, including molecules having a radioactive isotope or a chemical adduct such as a fluorophore, chromophore or biotin (“label”). Thus the nucleic acid may comprise modified nucleotides. Said modifications include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate and phosphoroamidate. The term “nucleic acid molecule” specifically includes single and double stranded forms of DNA and RNA.

It will be appreciated by those of ordinary skill in the art that, as a result of the degeneracy of the genetic code, there are many nucleotide sequences that may encode any given oligopeptidic compound as described herein. By degenerate nucleotide sequences is meant two (or more) nucleotide sequences which encode the same protein (or protein sequence), specifically in the open reading frame of the reference nucleotide sequence which begins at position 1 (i.e. in which codon 1 of the encoding sequence corresponds to positions 1-3 of the reference nucleotide sequence).

Also provided by the invention is a construct comprising a nucleic acid molecule of the invention. Thus the invention provides a construct comprising a nucleotide sequence that encodes an oligopeptidic compound of the invention.

The construct is conveniently a recombinant construct comprising the nucleic acid molecule of the invention. In the construct, the nucleic acid molecule of the invention may be flanked by restriction sites (i.e. nucleotide sequences recognised by one or more restriction enzymes) to enable easy cloning of the nucleic acid molecule of the invention. In the construct of the invention the nucleotide sequence encoding the oligopeptidic compound of the invention may conveniently be operably linked within said construct to an expression control sequence. The expression control sequence may be a human or non-human expression control sequence. The expression control sequence may be a mammalian expression control sequence, e.g. a murine or hamster expression control sequence. The expression control sequence may be derived from a lower eukaryote, e.g. yeast, or may be a prokaryotic, e.g. bacterial expression control sequence. Such an expression control sequence is typically a promoter, though the nucleotide sequence encoding the oligopeptidic compound may alternatively or additionally be operably linked to other expression control sequences such as a terminator sequence, an operator sequence, an enhancer sequence or suchlike.

The term “operably linked” refers to the association of two or more nucleic acid molecules on a single nucleic acid fragment so that the function of one is affected by the other. For example, a promoter is operably linked to a coding sequence when it is capable of affecting the expression of that coding sequence (i.e. when the coding sequence is under the transcriptional control of the promoter). Coding sequences may be operably linked to regulatory sequences in sense or antisense orientation.

The term “expression control sequence” refers to nucleotide sequences located upstream (5′ non-coding sequences), within, or downstream (3′ non-coding sequences) of a coding sequence, and which influence transcription, RNA processing or stability, or translation of the associated coding sequence. Expression control sequences may include promoters, operators, enhancers, translation leader sequences, a TATA box, a B recognition element and suchlike. As used herein, the term “promoter” refers to a nucleotide sequence capable of controlling the expression of a coding sequence or RNA. Suitable examples are well-known in the art. In general, a coding sequence is located 3′ to a promoter sequence. Promoters may be derived in their entirety from a gene, or be composed of different elements derived from different promoters found in nature, or even comprise synthetic nucleotide segments. It is further recognised that since in most cases the exact boundaries of regulatory sequences have not been completely defined, nucleic acid fragments of different lengths may have identical regulatory activity.

Methods for preparing a construct of the invention are well known in the art, e.g. conventional polymerase chain reaction (PCR) cloning techniques can be used to construct the nucleic acid molecule of the invention which may be inserted into suitable constructs (e.g. containing an expression control sequence) using known methods.

The invention further provides a vector comprising the nucleic acid molecule or construct of the invention. The term “vector” as used herein refers to a vehicle into which the nucleic acid molecule or construct of the invention may be introduced (e.g. be covalently inserted) and from which the oligopeptidic compound or mRNA encoding it may be expressed and/or the nucleic acid molecule/construct of the invention may be cloned. The vector may accordingly be a cloning vector or an expression vector. The vector of the invention thus comprises a nucleotide sequence encoding an oligopeptidic compound of the invention.

The nucleic acid molecule or construct of the invention may be inserted into a vector using any suitable methods known in the art, for example, without limitation, the vector and nucleic acid molecule may be digested using appropriate restriction enzymes and then may be ligated with the nucleic acid molecule having matching sticky ends, or as appropriate the digested nucleic acid molecule may be ligated into the digested vector using blunt-ended cloning.

The vector may be a bacterial or prokaryotic vector, or it may be a eukaryotic vector, e.g. a mammalian vector. The nucleic acid molecule or construct of the invention may be produced in or introduced into a general purpose cloning vector, particularly a bacterial cloning vector, e.g. an Escherichia coli cloning vector. Examples of such vectors include pUC19, pBR322, pBluescript vectors (Stratagene Inc.) and pCR TOPO® from Invitrogen Inc., e.g. pCR2.1-TOPO. The nucleic acid molecule or construct of the invention may be sub-cloned into an expression vector for expression of the oligopeptidic compound of the invention. Expression vectors can contain a variety of expression control sequences. In addition to control sequences that govern transcription and translation, vectors may contain additional nucleic acid sequences that serve other functions, including for example vector replication, selectable markers etc. Vector design is well known in the art.

Examples of vectors are plasmids, autonomously replicating sequences, and transposable elements. Additional exemplary vectors include, without limitation, phagemids, cosmids, artificial chromosomes such as yeast artificial chromosome (YAC), bacterial artificial chromosome (BAC), or PI-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g. herpes simplex virus), poxvirus, baculovirus, papillomavirus, and papovavirus (e.g. SV40).

The skilled person will be well aware of suitable vectors for use in the invention and of the elements they require for use.

In a final aspect the invention provides a method of downregulating expression of IFNβ by a human cell, the method comprising contacting the cell with an oligopeptidic compound of the invention. Generally, the method comprises contacting the cell with an oligopeptidic compound of the invention which is capable of blocking signalling from TLR4, TLR7, TLR8 and/or TLR9. This method may be performed in vitro or ex vivo, e.g. upon a human cell line or primary human cells obtained from a donor. The human cell may in particular be an immune cell, such as a monocyte, macrophage or dendritic cell. Contacting of the cell with an oligopeptidic compound of the invention may also downregulate expression of one or more of the pro-inflammatory cytokines IL-1β, TNF, IL-6 and CXCL10.

Down-regulation of IFNβ may occur only in the context of activation of a TLR, particularly TLR4. The same applies to down-regulation of the expression of the other cytokines. Thus down-regulation of the expression of the cytokines may be performed by applying both an oligopeptidic compound of the invention and a TLR4 ligand (e.g. LPS) to the cells. The oligopeptidic compound and TLR4 ligand may be applied to the cells in either order, though application of the oligopeptidic compound to the cells prior to application of the TLR4 ligand may be advantageous. Thus this method may be for downregulating expression of IFNβ by a human cell in response to contacting of the cell with a TLR4 ligand. Similarly the method may be performed to downregulate expression of IL-1β, TNF, IL-6 and/or CXCL10 in response to contacting of the cell with a TLR4 ligand.

Measurement of the expression levels of the cytokines, e.g. IFNβ, may be performed by any method known in the art. Suitable methods include quantitative PCR (qPCR) and ELISA (demonstrated in the Examples). Expression of a cytokine, such as IFNβ, is achieved if the level of expression of the cytokine following contacting of the cells with the oligopeptidic compound is lower than in control cells to which the oligopeptidic compound was not applied, but which are otherwise treated identically to the cells contacted with the oligopeptidic compound. The oligopeptidic compound may be applied to the cells at any suitable concentration to achieve downregulation of IFNβ. The necessary concentration may be dependent on the oligopeptidic compound used, and can be determined empirically.

This method of the invention may, for instance, be used in research to further elucidate cellular mechanisms and activity in response to TLR4 activation.

The invention may be further understood by the following non-limiting Examples and Figures:

### Figure Legends

FIG. 1 shows screening of single and double SLAMF1 mutants, to identify amino acids that are critical for the SLAMF1-TRAM interaction. (A-D) show the results of co-precipitations of SLAMF1^{Flag} and TRAM^{YFP} from lysates following their overexpression in HEK 293T cells. Co-precipitation was performed using anti-Flag antibodies. Anti-GFP Western blotting was performed to visualise TRAM^{YFP}, anti-Flag Western blotting to detect SLAMF1^{Flag} (including mutated SLAMF1). Presence of TRAM in precipitate indicates co-precipitation with and thus binding to SLAMF1. All experiments were performed at least 3 times and representative blots shown. GFP/FLAG ratios are shown for each co-precipitation experiment. The GFP/FLAG ratio corresponds to the TRAM/SLAMF1 ratio, i.e. a higher ratio indicates greater co-precipitation of SLAM F1, and thus enhanced binding of SLAM F1 to TRAM. GFP/FLAG ratios were calculated based on the bands' intensities, quantified using Odyssey LI-COR software.

FIG. 2 shows the selection and size optimisation of ECFP-tagged peptides in cell-free immunoprecipitation assays. (A) Western blot analysis of SLAMF1 and TRAM^{Flag} co-precipitated in the presence of CFP-tagged peptides. TRAM^{Flag} was precipitated from cell lysates on agarose beads and distributed across 15 tubes. Equal amounts of SLAMF1-containing lysates and a normalised amount of ECFP or ECFP-tagged peptide (normalisation demonstrated in “Lysates” blot) were added to TRAM^{Flag}-agarose, and incubated with rotation at +4° C. for 4 h. Unbound proteins/peptides were removed by a washing step, and pecipitates analysed by WB (anti-Flag IPs). Reduced SLAMF1 binding to TRAM indicates inhibition of the TRAM/SLAMF1 interaction by the peptide. (B) Sequences of the peptides that were expressed as ECFP-tagged proteins (N-terminal ECFP). Peptides 4 and 5 are not shown and were not pursued.

FIG. 3 shows that Pep7-Arg11 inhibits IFNβ, TNF and CXCL10 mRNA expression in a concentration dependent manner. (A) LDH cell death assay. (B-D) qPCR with TaqMan probes to determine IFNβ, TNF and CXCL10 expression levels after LPS stimulation. Cells were pre-treated with peptides for 1 h before LPS (100 ng/ml) stimulation. (E) Western blot analysis for phospho-STAT1 with β-tubulin as endogenous control, showing that at higher concentrations (10 and 20 μM) Pep7-Arg11 strongly inhibited IFNβ/IFNAR-mediated phosphorylation of STAT1 upon LPS stimulation of THP-1 cells.

FIG. 4 shows that both Pep7-Arg11 and Pep7-penetratin reduced IFNβ (A,B) and CXCL10 (C,D) mRNA expression by THP-1 cells, with much less background toxicity seen for Pep7-Penetratin than for Pep7-Arg11 (E). Cells were pre-treated for 30 min with media containing DMSO-diluted TAMRA-linked peptides or water-diluted peptides with different CPPs (Arg11 or penetratin) at 10 μM concentration.

FIG. 5 shows multiplex data demonstrating the effect of peptides on cytokine production by THP1 cells in response to LPS stimulation. All peptides used are from water-diluted stocks. Before stimulation cells were pre-treated for 30 min with 10 μM peptides in culture media.

FIG. 6 shows that P7 peptides with different CPPs (Arg11 or penetratin) very effectively decreased TNF and IFNβ mRNA expression in primary human monocytes stimulated by LPS (A/C) or E. coli (B/D). The peptides' effect on cell viability is shown in (E).

FIG. 7 shows the results of screening for P7 efficacy in down-regulating cytokine production using different CPPs and CPP positions (N-terminus vs. C-terminus) in THP-1 cells. (A) Results of LDH assay and ELISAs analysing cytokine secretion after 5 h of stimulation with LPS (graphs show mean values for at least 3 biological replicates). (B) Tables present heat maps for the ratio of cytokine expression levels or LPS-induced cell death for the cells treated by peptides/control treated cells (treated with solvents, i.e. water or DMSO), i.e. values below 1 indicate reduced cell death (for LDH assay) or reduced cytokine production, and values above 1 indicate increased cell death or increased cytokine production. In the tables average values are presented based on 3-5 biological replicates for each experiment. Cells were pretreated with 15 μM peptide or solvent in culture media for 30 min before addition of LPS (100 ng/ml) and collection of SNs for ELISA or LDH assays.

FIG. 8 shows the results of screening for the effect of CPP positioning at the N- or C-terminus of the Pep7 peptide on the secretion of pro-inflammatory cytokines TNF and IL-113. The figure shows ELISA data for the secretion of cytokines by THP-1 cells pre-treated with 10 μM peptides for 30 min, followed by stimulation with LPS (100 ng/ml) for 4 h. Error bars demonstrate SD for 6 biological replicates.

FIG. 9 shows that the Pep3-Arg11 peptide is not effective in inhibiting LPS-induced cytokine mRNA expression. THP-1 cells were stimulated with LPS for increasing periods of time. Cell viability was determined by LDH assay (A). The figure shows qPCR data for IFNβ (B), TNF (C) and CXCL10 (D) mRNA expression, and Western blot analysis of STAT1 phosphorylation levels in the lysates after simultaneous RNA/protein extraction (E). 15 μM peptides (diluted in water) were used for pre-treatment of cells before stimulation. The figure shows the results of one representative experiment (out of 3 performed).

FIG. 10 shows the results of screening for efficacy of extension of the P7 peptide at the N- and/or C-terminus. (A) shows LDH assay results for cell viability and ELISA results for cytokine secretion after 5 h of stimulation of THP-1 cells with LPS (the graphs show the mean values for at least 3 biological replicates). (B) shows heat maps for the ratio of cytokine expression levels or LPS-induced cell death for cells treated with peptides/control treated cells (treated with solvents, water or DMSO). In the tables average values are presented for at least 3 experiments with 3-5 biological replicates for each experiment. Cells were pretreated with 15 μM peptides or solvent in culture media for 30 min before addition of LPS (100 ng/ml) and collection of SNs for ELISA or LDH assays.

FIG. 11 shows the results of screening for functional amino acid substitutions in the P7 lead peptide sequence. (A) shows LDH assay results for cell viability and ELISA results for cytokine secretion after 5 h of stimulation of THP-1 cells with LPS (the graphs show the mean values for at least 3 biological replicates). (B) shows heat maps for the ratio of cytokine expression levels or LPS-induced cell death for cells treated with peptides/control treated cells (treated with solvents, water or DMSO). In the tables average values are presented for at least 3 experiments with 3-5 biological replicates for each experiment. Cells were pretreated with 15 μM peptides or solvent in culture media for 30 min before addition of LPS (100 ng/ml) and collection of SNs for ELISA or LDH assays.

FIG. 12 shows that the P7 peptide is very effective in blocking the IFNβ-CXCL10 signaling axis when added to THP-1 cells prior to or simultaneously with LPS, but not as effective when added to the cells 1 hour after the start of LPS treatment. Cells were treated with 10 μM peptides at designated time points and stimulated with LPS (100 ng/ml) for 6 h.

FIG. 13 shows that the P7 peptide with Pen or KLA CPP and P7-A4-Pen do not alter cell viability (A), and very effectively down-regulated Ifnβ (B) and pro-inflammatory cytokine (C,D) mRNA expression in B6 murine immortalized macrophages. Cells were pre-treated with 10 μM peptides in cell culture media for 1 h, and stimulated with LPS (100 ng/ml) for increasing lengths of time. The figure shows the results of one representative experiment (out of 3 performed).

FIG. 14 shows the results of screening for the effect of CPP positioning (N-terminus vs. C-terminus) on P7 efficacy in inhibiting TLR4-mediated cytokine expression in B6 murine immortalized macrophages. The figure shows qPCR data for mRNA expression of Ifnβ (A), Tnf (B) and II-1β (C). Cells were pre-treated with 10 μM peptides in cell culture media for 1 h, and stimulated with LPS (100 ng/ml) for increasing lengths of time. The P7C3-Pen peptide was used as a control along with solvent controls (water for C3-Pen and P7-Pen and DMSO for Pen-P7). The figure shows the results of one representative experiment (out of 3 performed).

FIG. 15 shows that pre-treatment of primary human monocytes with P7-Pen peptide inhibited both IFN/3 and pro-inflammatory cytokine mRNA expression mediated by TLR4 (A), and IFN/3 expression mediated by TLR8 (B). Cells were pre-treated with 15 μM peptides for 30 min, followed by addition of LPS (100 ng/ml) (A) or CL075 (1 μg/ml) (B) for 2 or 4 h. Each dot on the graphs corresponds to the average value for one donor, the lines show the mean values for all donors, and error bars indicate the SD.

FIG. 16 shows that the P7-Arg11 peptide inhibited LPS, E. coli and CL075-mediated IFNβ secretion by whole blood cells. The figure shows the results of a pilot assay, in which cells were pre-treated with 10 μM control (Arg11) or P7-Arg11 peptides, followed by stimulation with LPS (100 ng/ml), E. coli particles (7×10⁶/ml) or CL075 (1 μg/ml). ELISA data, mean values for 2 biological replicates from one donor.

FIG. 17 shows that P7-Pen inhibited TLR4- and TLR8-mediated IFNβ release by whole blood cells, with a differential effect on the secretion of pro-inflammatory cytokines. The figure shows ELISA data for secretion of IFNβ and bioplex assays for detection of pro-inflammatory cytokines in response to LPS (100 ng/ml) (A), CL075 (1 μg/ml) (B), E. coli particles (7×10⁶/ml) (C) or S. aureus particles (3.5×10⁶/ml) (D). Whole blood samples from healthy donors were pre-treated with 20 μM peptides for 1 h then stimulated with TLR ligands for 4 h before plasma collection. Results are for at least 6 donors. The Wilcoxon matched-pairs signed rank test was used for statistical analysis between 2 groups in Prizm 8.2.1. In each graph, circles indicate whole blood pre-treated with water, squares indicate blood pre-treated with control peptide (average for treatment with Pen and C3-Pen), and triangles indicate pre-treatment with P7-Pen.

FIG. 18 shows that pre-treatment of primary human monocytes with P7-Pen peptide inhibited mRNA expression of IFN/3 and pro-inflammatory cytokines mediated by the TLR4 ligand LPS (A), and IFN/3 expression mediated by TLR8 and 7 ligands (B,C top panels), but had no effect on TLR7- and 8-mediated pro-inflammatory cytokine expression (B,C lower panels). The figure shows qPCR data with specific TaqMan probes. Cells were pre-treated with 15 μM peptides for 30 min, followed by the addition of LPS (100 ng/ml) (A), R837 (1 μg/ml) (B) or CL075 (1 μg/ml) for increasing lengths of time.

FIG. 19 shows that the P7-Pen peptide showed no toxicity (A) and high efficacy in inhibiting TLR9-mediated mRNA expression of IFNβ (B), CXCL10 (C) and the pro-inflammatory cytokines TNF (D) and IL-1β (E) in undifferentiated THP-1 TLR9^{CHERRY} cells. CpG 2006 (10 μM) was used as specific TLR9 ligand. (A) Results of LDH assay on SNs from stimulated cells. (B-E) Results of qPCR analysis of cytokine expression with TaqMan probes. (F) Results of ELISA for secreted IL-113 levels. Cells were pre-treated with 15 μM peptides for 1 h, followed by addition of CpG 2006 to the cell culture media.

FIG. 20 shows that the P7-pen peptide inhibits uptake of E. coli and S. aureus particles by human cells. Cells were pre-incubated with Pen or P7-Pen peptides, followed by addition of Alexa Fluor 488-labeled bioparticles for the times indicated (15, 30 or 45 min). Fluorescent bacteria particles were counted by spot-detection in Bitplane Imaris imaging software and used to calculate the number of particles per cell. (A) shows uptake of E. coli by THP-1 WT cells or THP-1 cells overexpressing TRAM^{CHERRY.} (B) shows uptake of E. coli and S. aureus particles by THP-1 TRAM^{CHERRY} cells. (C) shows uptake of E. coli by primary human monocytes. Pre-incubation with 3 μM cytochalasin D (CytoD, and inhibitor of actin polymerization) inhibited uptake of E. coli to a similar extent as P7-Pen.

FIG. 21 shows that TRAM is not required for II-β mRNA expression in response to LPS and E. coli by human THP-1 macrophages. The figure shows qPCR analysis of cytokine mRNA expression by control, TRAM^{-/-} and TLR4^{-/-} THP-1 cells stimulated with LPS (100 ng/ml) (A) or E. coli particles (B).

FIG. 22 shows that LPS-mediated TNF and IL-1β mRNA expression is efficiently reduced by Pep7-Penetratin (P7-Pen) in TRAM KO cells, indicating that reduction of TNF and IL-18 levels in Pep7-treated cells could be mediated by targeting a protein other than TRAM. Control THP-1 CRISPR cells and TRAM KO cells were pre-treated with 10 μM peptides—control peptides with amino acid substitutions (C3-Pen) or TRAM-targeting peptide Pep7-Penetratin (P7-Pen)—for 30 min before stimulation with LPS (100 ng/ml) for 2 or 4 h. Cells were lysed with Qiazol, followed by RNA isolation and quantitative PCR analysis of cytokine mRNA expression.

FIG. 23 shows that pretreatment of murine immortalized BMDMs with Pep7-Penetratin (P7-Pen) peptide before LPS stimulation not only decreased intracellular TLR4-mediated signaling (less phospho-TBK1), but also altered TLR4 signaling from the cell surface. Western blot analysis of lysates from cells pretreated with media containing controls (water, DMSO or 10 μM control peptide C3-Pen) or P7-Pen peptide for 30 min before stimulation with LPS (K12 LPS, 100 ng/ml) for designated time periods.

FIG. 24 shows that pretreatment of macrophages with P7-Pen peptide before LPS stimulation delays/abrogates polyubiquitination of IRAK1, degradation of IκBα and phosphorylation of p-p38MAPK. A shows the effect on THP-1 cells. B shows the effect on primary human monocytes. The figure shows Western blot analysis of lysates from cells pretreated with control peptides Pen and C3-Pen, or P7-Pen peptide for 30 min before stimulation with LPS (K12 UP LPS, 100 ng/ml) for designated time periods.

FIG. 25 shows that the P7 peptide does not affect phosphorylation of p38 MAPK or TBK1 or expression of pro-inflammatory cytokines IL-6, IL-1β and TNF induced by TLR2 or TLR8 ligation in human macrophages. Primary human macrophages (PBMC derived, differentiated in media with 10% human serum and M-CSF 20 ng/ml, 7 days) were stimulated with K12 LPS (100 ng/ml), FSL-1 (100 ng/ml) or CL075 (3 μg/ml). (A) shows Western blot analysis of lysates of stimulated cells, PCNA used as loading control. (B) qPCR for mRNA expression of IFNβ, IL-6, TNF and IL-1β, average for three biological replicates, error bars ±SD.

FIG. 26 shows that biotinylated P7-Pen peptide on NeutrAvidin beads precipitated several signaling molecules: TRAM, Mal, MyD88, IRAK1 and IRAK4, and after LPS stimulation TRAF6 and TAK1 as well. Precipitations were performed using biotinylated peptides on lysates of primary human macrophages stimulated with LPS (100 ng/ml) for 15, 30 or 60 min and unstimulated cells (A), and stimulated with LPS for 30 or 60 min, or CL075/TLR8 ligand for 15 or 30 min (B).

FIG. 27 shows that Mal co-precipitates with P7-Pen peptide and SLAMF1 protein, and shows the endogenous Mal interaction with the MyD88 signaling complex. Interactions between Mal and MyD88 and IRAK1 were totally abrogated in P7-Pen pre-treated human macrophages. (A) Mal^{Flag} was overexpressed in HEK293T cells. Cell lysis followed by precipitations with biotinylated Pen (control) or P7-Pen showed specific co-precipitation with P7-Pen. Lysis buffer contained 300 mM NaCl and 1% Triton X100. (B) Mal^{Flag} and SLAMF1 wt or deletion mutants (Δ20 or Δ67 C-terminal amino acids) were co-expressed in HEK293T cells followed by immunoprecipitations with anti-Flag beads and Western blot analysis. (C) Primary human macrophages were pre-treated with 15 μM peptides for 30 min, stimulated with LPS for another 30 min or 1 h, lysed and used for IPs. Precipitations were performed using goat anti-Mal polyclonal antibodies covalently bound to magnetic beads, followed by Western blot analysis. Proteins in lysates were analysed for input control (A-C).

FIG. 28 shows that pre-treatment of cells with P7-Pen peptide abrogated IRAK1, 2 and 4 recruitment to the MyD88 signaling complex in human monocytes. Cell were pre-treated with Pen or P7-Pen (15 μM) for 30 min before addition of LPS (100 ng/ml) for specified time periods. Precipitations were performed using sheep anti-IRAK4 (A), anti-IRAK1 (B) and anti-IRAK2 (C) polyclonal antibodies covalently bound to magnetic beads, followed by Western blot analysis of lysates and precipitated proteins.

FIG. 29 shows that SLAMF1 co-precipitated with ubiquitinated/phosphorylated IRAK1, but not IRAK2, after LPS stimulation of primary human macrophages. The SLAMF1 and TRAM interaction was used as a positive control. Primary human macrophages were pre-treated with 15 μM peptides for 30 min, stimulated with LPS for another 15′, 30′ or 1 h, lysed and used for co-precipitation with anti-SLAMF1 mouse mAbs (E-11) covalently bound to magnetic beads, followed by Western blot analysis. Proteins in lysates were analysed as input control.

FIG. 30 shows that the P7-Pen peptide had no effect on TLR2-mediated IRAK1 modification and IRAK1 and 4 recruitment to MyD88, but abrogated TLR4-mediated IRAK recruitment to MyD88, and decreased early recruitment of IRAKs upon TLR8 stimulation. Primary human macrophages (7 days) were pre-treated with Pen or P7-Pen (15 μM) for 30 min before addition of LPS (100 ng/ml), FSL-1 (100 ng/ml) or CL075 (3 μg/ml). A: Protein expression in lysates for input control. B,C: Precipitations were performed using sheep anti-IRAK4 (B) or anti-IRAK1 (C) polyclonal antibodies covalently bound to magnetic beads.

FIG. 31 shows that overexpression of IRAK4 with Mal targets Mal for degradation (A), and the effect of IRAK4^{EGFP} overexpression is partially inhibited by ECFP-P7 co-expression (B).

FIG. 32 shows that incubation of multiple myeloma cells from the INA-6, JJN-3, ANBL-6, IH-1 and RPMI 8226 lines with P7-A4-Pen inhibits proliferation of the INA-6, JJN-3, ANBL-6 cell lines, and induces cell death for the JJN-3 cell line. Minor effects on the proliferation of the IH-1 and RPMI 8226 cell lines are seen. Cells were seeded at the level shown (left-hand bar of each set) then incubated in medium containing water, 10 μM penetratin or 10 μM P7-A4-Pen for 48 hr (except for INA-6 cells, which were incubated with 12.5 μM penetratin or 12.5 μM P7-A4-Pen). The number of viable cells was detected based on quantitation of ATP by luminescent signal in correlation to a standard curve prepared for each cell line. Results are based on 3-5 biological replicates for each condition, error bars show standard deviation of mean.

FIG. 33 shows the results of screening for functional sequence modifications relative to the P7 lead peptide sequence. (A) shows LDH assay results for cell viability and ELISA results for cytokine secretion after 5 h of stimulation of THP-1 cells with LPS (the graphs show the mean values for 3 biological replicates). (B) shows heat maps for the ratio of cytokine expression levels or LPS-induced cell death for cells treated with peptides/control treated cells (treated with solvents, water or DMSO). Cells were pretreated with 15 μM peptides or solvent in culture media for 30 min before addition of LPS (100 ng/ml) and collection of SNs for ELISA or LDH assays.

FIG. 34 shows the effect on proliferation of cells of the ANBL-6 multiple myeloma line of incubation with 39 SLAMF1-derived peptides in the context of penetratin conjugates. Cells were seeded and then incubated in growth medium alone or supplemented with water, DMSO, 15 μM penetratin or 15 μM investigational peptide. The growth medium only and H₂O—, DMSO- and penetratin-supplemented cultures constituted controls. The level of cell growth varied between treatments, but 5 peptides were found to display a clear anti-proliferative effect on the ANBL-6 cells: P7-K2-Pen, P7-A4-Pen, P7-N4-Pen, P7-D4-Pen and P7-S4-Pen.

FIG. 35 shows the effect of putative anti-proliferative peptides on ANBL-6 multiple myeloma cells, when applied at a range of concentrations. The solid horizontal lines show the amount of cells originally seeded. In (A) ANBL-6 cells were seeded at a level of 10,000 cells/well, then incubated in medium supplemented with water, the control peptide C3A-Pen, P7-Pen, P7-A4-Pen, P7-N4-Pen or P7-K2-Pen for 48 hours. The peptides were included at concentrations of 5, 10, 12.5, 15, 20 or 30 μM. Cells were counted at 48 hours. In (B), ANBL-6 cells were seeded at a level of 5000 cells/well, then incubated in medium supplemented with water, P6-Pen, P-P6-Pen, P7-D4-Pen or P7-A4-Pen for 48 hours. Peptides were included at concentrations of 5, 10 or 20 μM. Cells were counted at 48 hours. P7-A4-Pen, P7-N4-Pen, P7-D4-Pen and P7-K2-Pen showed clear concentration-dependent anti-proliferative/cytotoxic activity. Results are based on 3 biological replicates, error bars show standard deviation of mean.

FIG. 36 shows the effect of combining the alkylating chemotherapy agent melphalan with P7-A4-Pen and P7-N4-Pen on the proliferation of multiple myeloma cells. In (A) ANBL-6 cells were seeded and incubated for 48 hours in medium supplemented with ethanol (solvent for melphalan), ethanol plus 5 μM P7-A4-Pen, melphalan at 8, 10, 12, 16 or 20 μM, or 5 μM P7-A4-Pen plus melphalan at the same set of concentrations. In (B) JJN-3 cells were seeded and incubated for 48 hours in medium supplemented with ethanol, ethanol plus 4 μM P7-N4-Pen, melphalan at the above-listed concentrations or 4 μM P7-N4-Pen plus melphalan at the same set of concentrations. Cells were counted after 48 hours. As shown, the combination of melphalan plus P7-A4-Pen or P7-N4-Pen displayed enhanced cytotoxicity relative to either compound alone. Results are based on 3 biological replicates, error bars show standard deviation of mean.

FIG. 37 shows the effect of various peptides on the viability of the acute myeloid leukaemia cell line THP-1. THP-1 cells were seeded in medium at 10,000 cells/well, as indicated by the dotted line. Cells were incubated for 48 hours in medium supplemented with water, C3A-Pen, P7-Pen, P7-A4-Pen or P7-N4-Pen. The peptides were included at concentrations ranging from 5-30 μM. At 48 hours cells were counted. P7-N4-Pen showed a clear dose-dependent cytotoxic effect on the THP-1 cells. The results with other peptides were less clear-cut. Results are based on 3 biological replicates, error bars show standard deviation of mean.

FIG. 38 shows the effect of the peptides P7-Pen and P7-N4-Pen on the proliferation of Jurkat T cell leukaemia cells. Jurkat cells were seeded at 5000 cells/well (as indicated by dotted line) and incubated in medium supplemented with water, penetratin, P7-Pen or P7-N4-Pen. Peptides were included at 15 μM (A) or 20 μM (B). Cells were incubated for 48 hours then counted. A modest but clear anti-proliferative effect was seen for P7-N4-Pen, and a possible anti-proliferative effect for P7-Pen. Results are based on 3 biological replicates, error bars show standard deviation of mean.

FIG. 39 shows the effect of the peptides P7-Pen and P7-N4-pen on the proliferation of SW480 colon cancer cells. SW480 cells were seeded and incubated in medium alone or supplemented with water, 15 μM penetratin, 15 μM P7-Pen or 15 μM P7-N4-Pen. Under each condition cells were incubated both without LPS and with 100 ng/ml LPS. Cells were incubated for 48 hours then counted. Both P7-Pen and P7-N4-Pen showed a modest anti-proliferative effect. The presence of LPS made no difference to the effect of the peptides on proliferation. Results are based on 3 biological replicates, error bars show standard deviation of mean.

FIG. 40 shows the effect of the peptides P7-Pen, P7-A4-Pen and P7-N4-Pen on primary Vk*MYC multiple myeloma cells. Isolated primary cells were seeded then incubated for 48 hours in growth medium supplemented with water, penetratin, P7-Pen, P7-A4-Pen or P7-N4-Pen, then counted. Peptides were included at concentrations of 7.5 or 15 μM. P7-Pen, P7-A4-Pen and P7-N4-Pen all showed clear dose-dependent cytotoxic effects on the cells. Results are based on 3 biological replicates, error bars show standard deviation of mean.

FIG. 41 shows that the anti-proliferative peptides of the invention are not toxic to healthy blood cells. PBMCs were isolated from 3 donors (D1, D2 and D3) then incubated for 48 hours in growth medium supplemented with water, penetratin, P7-Pen or P7-A4-Pen, then counted. The peptides were included at concentrations of 5, 10 or 20 μM. No particular reduction in PBMC cell number was seen in cells from any donor with any concentration of peptide. Results are based on 3 biological replicates, error bars show standard deviation of mean.

FIG. 42 shows that the anti-proliferative peptide P7-A4-Pen induces apoptosis in the JJN-3 multiple myeloma cell line. JJN-3 cells were cultured for 24 or 48 hours with a control peptide (penetratin) or P7-A4-Pen, both at 10 μM. Cells were then lysed and lysates analysed by Western blot for cleaved caspase-3. Presence of cleaved (i.e. activated) caspase-3 is an indicator of apoptotic pathway activation. Cleaved caspase 3 was detected at high levels in the cells treated with P7-A4-Pen for 24 or 48 hours, and only at much lower levels in the cells treated with the control peptide. β-tubulin was detected as a loading control.

### EXAMPLES

Peptides were designed based on the information about interaction domains for SLAMF1 and TRAM proteins described above and published in Yurchenko et al. (supra). The amino acid sequence of the SLAMF1 protein was selected as basis to design peptides to target and inhibit the interaction of TRAM with SLAMF1 (such that the peptides would compete with endogenous SLAMF1 protein for the SLAMF1 binding region of TRAM), in order to inhibit synthesis of pro-inflammatory cytokines.

Materials & Methods

Primary Human Monocytes

Use of human monocytes from blood donors was approved by the Regional Committees for Medical and Health Research Ethics at the Norwegian University of Science and Technology (NTNU). Human monocytes were isolated from buffy coats by adherence as previously described (Husebye et al., Immunity 33: 583-596, 2010). Briefly, freshly prepared buffy coat (The Blood Bank, St. Olav's Hospital, Trondheim, Norway) was diluted in 100 ml PBS, and PBMCs isolated by density gradient centrifugation using Lymphoprep (Axis-Shield) according to the manufacturer's instructions. PBMCs were collected and washed with Hank's Balanced Salt Solution (Sigma-Aldrich, USA) four times by low speed centrifugation (150-200× g). Cells were counted using a Z2 Coulter Particle Count and Size Analyzer (Beckman Coulter) on program B, re-suspended in RPM11640 (Sigma) supplemented with 5% pooled human serum at a concentration of 8×10⁶ per ml and seeded into 6-well (1 ml per well) or 24-well (0.5 ml per well) cell culture dishes. Following a 45 min incubation, allowing surface adherence of monocytes, the dishes were washed 3 times with Hank's Balanced Salt Solution to remove non-adherent cells. Monocytes were maintained in RPM11640 supplemented with 10% pooled human serum (The Blood Bank, St Olav's Hospital) and used within 24 h after isolation.

Cell Lines

SW480 (ATCC CCL-228) and Jurkat (ATCC Clone E6-1; TIB-152) cells were cultured in RPMI 1640 (Sigma-Aldrich, Schnelldorf, Germany) supplemented with 10% heat-inactivated FCS, penicillin/streptomycin (Life Technologies) and 20 mM L-Glutamine. THP-1 cells (ATCC TIB-202) were cultured in RPMI-1640 (high-glucose, ATCC) supplemented with 10% heat inactivated foetal calf serum (FCS), 100 nM penicillin/streptomycin and 5 μM β-mercaptoethanol (Sigma). THP-1 cells were differentiated with 60 ng/ml phorbol 12-myristate 13-acetate (PMA, Sigma) for 72 h, followed by 24 or 48 h in medium without PMA.

Multiple myeloma cells JJN-3 (DSMZ (Germany) no. ACC 541), ANBL-6 (Cellosaurus accession number CVCL_5425, obtained from Dr Diane Jelinek, Mayo Clinic, Rochester, Minn.), INA-6 (Cellosaurus accession number CVCL_5209, obtained from Dr M. Gramatzki, University of Erlangen-Nurnberg, Erlangen, Germany), RPMI-8226 (ATCC CCL-155), and IH-1 (Cellosaurus accession number CVCL_WZ44, established from the pleural effusion of a myeloma patient by Myeloma research group, IKOM, NTNU, Trondheim, Norway) were grown in RPMI-1640 (Sigma-Aldrich Norway, Oslo, Norway) supplemented with 100 nM penicillin/streptomycin and 10% FCS, and for ANBL-6, IH-1 and INA-6 cells 2 ng/ml recombinant IL-6 (Gibco, Thermo Fisher Scientific, Waltham, Mass., USA). Murine multiple myeloma Vk*MYC cells were obtained from M.D. Leif Bergsagel (Mayo Clinic, Richester, Minn., USA) and cultured in RPMI-1640 supplemented with 2% human serum (B⁺, The blood bank, St Olav's Hospital, Trondheim, Norway), 100 nM penicillin/streptomycin, L-Glutamine (2 mM) and recombinant IL-6 (1 ng/ml). HEK293T (ATCC CRL-3216) were cultured in DMEM with 10% FCS. THP-1 TRAM knockout (KO) and TLR4 KO cell lines, and matching lentivirally transduced control cell lines, were differentiated in the same way as wild type (WT) THP-1. TRAM KO THP-1 cell line is described in Yurchenko et al. (supra).

To generate the TLR4 KO cell line, the LentiCRISPRv2 plasmid (Sanjana et al., Nature Methods 11: 783-784, 2014; Addgene #52961) was ligated with 5′-CACCGCCAGCTTTCTGGTCTCACGC-3′ (SEQ ID NO: 100) and 5′-AAACGCGTGAGACCAGAAAGCTGGC-3′ (SEQ ID NO: 101) to target TLR4. For the control cell line, the LentiCRISPRv2 plasmid was ligated with 5′-CACCGTTTGTAATCGTCGATACCC-3′ (SEQ ID NO: 102) and 5′-AAACGGGTATCGACGATTACAAAC-3′ (SEQ ID NO: 103) (which have no specific targets in the human genome). Packaging plasmids pMD2.G and psPAX2 were used for producing lentivirus (Addgene plasmids #12260 and #12259). HEK293T cells were co-transfected with the packaging and lentiCRISPRv2 plasmids and washed after 16 h. The lentivirus-containing supernatants were collected after 48 h and used for transduction of THP-1 cells along with 8 μg/ml protamine sulphate. Transduced THP-1 cells were then selected with Puromycin (1 μg/ml) for 1 month. The control cell line was tested along with the THP-1 cell line in LPS stimulations and did not demonstrate differences in cytokine expression compared to THP-1 WT cells. The TLR4 KO cell line was, as expected, not sensitive to LPS-treatment, but has unaffected responses to the TLR2/6 ligand FSL-1 (as detailed below). All cell lines were regularly checked for mycoplasma contamination.

Immortalized bone-derived-macrophages (iBMDM) from wild type, Tram−/− or TIr4−/− C57BL/6 mice were maintained at 37° C. and 5% CO₂ in DMEM supplemented with 10% FCS.

Peripheral Blood Mononuclear Cells (PBMCs)

The use of PBMCs was approved by the Regional Committees from Medical and Health Research Ethics at the Norwegian University of Science and Technology (REK no. 2009/2245). Cells were isolated from buffy coats using Lymphoprep separation medium (Axis-Shield, Oslo, Norway) and washed with Hanks' Balanced Salt solution (Sigma Aldrich, Schnelldorf, Germany). Cells were resuspended in RPMI-1640 supplemented with 10% FCS, 2% human serum (A⁺, The Blood bank, St Olav's Hospital, Trondheim, Norway), 100 nM penicillin/streptomycin and L-Glutamine (2 mM). 50,000 cells/well of PBMCs were seeded in white-walled flat-bottomed 96-well plates in 200 μl of media and treated with media with solvent (water) or 20 μM peptides. PBMCs were kept in a total volume of 250 μl/well and incubated with treatment for 48 hr.

Whole Blood Assay

To evaluate the effect of peptides in an ex vivo system, a human whole blood model was used. Peripheral venous blood was drawn from 6 healthy donors into sterile polypropylene tubes (Thermo Scientific™ Nunc™ Biobanking and Cell Culture Cryogenic Tubes) containing the anti-coagulant Lepirudin (Refludan®; Celgene). Peptide solutions were made by diluting peptide stocks in DPBS (Dulbecco's Phosphate Buffered Saline with MgCl₂ and CaCl₂; Sigma-Aldrich) to obtain a final concentration of 140 μM. Peptide solution or DPBS was distributed in equal volume (50 μL) to 1.8 mL sterile polypropylene tubes (Thermo Scientific™Nunc™ Biobanking and Cell Culture Cryogenic Tubes). 250 μL whole blood was then added to the 1.8 mL tubes containing peptide solution or DPBS, and incubated at 37° C. under constant rotation for 1 h (concentration of peptides for pre-incubation: 23.3 μM). Stimulation solutions were made by diluting K12 LPS or CL075 in DPBS to obtain a final concentration in whole blood of 100 ng/mL (LPS) or 1 μg/mL (CL075) after addition of 50 μL stimulation solution or DPBS. Concentration of peptides in whole blood samples was reduced by this to 20 μM. Tubes were incubated at 37° C. under constant rotation for 4 h. Finally, plasma was isolated from the whole blood specimens by centrifugation, collected in 96 well plates (Corning® 96 Well CellBIND® Microplates), and stored at −20/−80° C. until analysis by multiplex/27-plex cytokine assay or IFNβ ELISA (VeriKine-HS Human Interferon Beta Serum ELISA Kit; pbl Assay Science).

Reagents and Cell Stimulation

pHrodo Red E. coli were purchased from Thermo Fisher Scientific. Ultrapure E. coli K12 LPS, polyinosinic-polycytidylic acid [poly(I:C)], and the thiazoloquinoline compound CL075, were obtained from InvivoGen. Ultrapure K12 LPS was used at a concentration of 100 ng/ml. E. coli bioparticles were reconstituted in 2 ml PBS, and 50 μl/well (1.5×10⁷ particles) in 1 ml of media was used for cells in 6-well plates (NUNC) or 35-mm glass bottom tissue cell dishes (MatTek Corp.), or 15 μl/well (0.45×10⁷ particles) in 0.5 ml of media for 24-well plates (NUNC).

Peptides

Peptides were synthesised to custom order as TFA-free (with TFA substitution to acetate ions), with N-terminal acetylation and C-terminal amidation, by GenScript. Peptides were dissolved in the suggested optimal solvent (DMSO or sterile milliQ water) to concentrations from 0.5 to 5 mM, aliquoted and stored at −80° C., avoiding freezing-unfreezing cycles. Peptides were dissolved to the working concentration in cell culture media (cell-based assays) or PBS (whole blood assays) just before use.

Antibodies

The following primary antibodies were used: rabbit-anti-TICAM-2/TRAM (GTX112785) from Genetex; rabbit anti-human SLAMF1/SLAMF1 (#10837-R008-50) from Sino Biological Inc.; anti-tubulin antibodies from abcam; IkB-α (44D4) #4812, phospho-IkB-α (14D4) #2859, phospho-p38 MAPK (Thr180/Tyr182) (D3F9) #4511, phospho-TBK1/NAK (Ser172) (D52C2) #5483, phospho-TAK1 (T184/187) (9007) #4508, TAK1 #5206, anti-DYKDDDDK Tag (D6W5B)/Flag-tag #14793, anti-MyD88 (D80F5) #4283, anti-IRAK1 (Human Specific) #4359, anti-IRAK1 (D51G7) #4504, anti-IRAK4 #4363, anti-TRAF6 (D21G3) #8028, phospho-STAT1 (Tyr701) (D4A7) #7649 from Cell Signaling; Living Colors rabbit anti-full-length GFP polyclonal Abs (#632592) from Clontech; 4G10® Platinum anti-phosphotyrosine antibody biotin conjugated (#16-452) from Merck-Millipore (Merck Life Science AS); mouse anti-Glutathione-S-Transferase (SAB4200237), monoclonal mouse ANTI-FLAG M2 antibodies (#F1804-200UG) from Sigma; rabbit anti-caspase-3 (#9662) from Cell Signaling Technology. Secondary antibodies (HRP linked) for Western blotting were swine anti-rabbit (P039901-2) and goat anti-mouse (P044701-2) from DAKO/Agilent.

Live Cell Imaging

Human primary monocytes were seeded into 24-well glass-bottomed plates (MatTek Corporation) in RPMI media containing 25% pooled human serum. The day after isolation, cells were washed once more with RPMI and media changed to RPMI supplemented with 10% human serum, followed by addition of peptides (Arg11 or Pep7-Arg11) labelled with N-terminal TAMRA (GenScript) in a sterile hood. Cells were immediately transferred to a microscope and peptide accumulation was followed using a ZEISS AiryScan microscope. Images were taken at different time points following accumulation of peptides inside the cells.

Confocal Microscopy Analysis

THP-1 cells, THP-1^{cherry} cells or human PBMC were seeded in 24-well glass-bottom plates (MatTek Corporation), 125,000 cells/well. The THP-1 cells were differentiated into macrophages with 60 ng/ml PMA for 72 h and thereafter rested for 48 h, whereas the PBMC were used the day after isolation. The P7-penatratin peptide or control peptide was added to the cells 30 min before stimulation at 15 μM concentration. If used, 3 μM cytochalasin D (CytoD) was added simultaneously. The cells were stimulated with Alexa Fluor 488-conjugated E. coli K-12 BioParticles or Alexa Fluor 488-conjugated S. aureus BioParticles (12 particles/cell). Prior to stimulation bacterial particles were sonicated and opsonised in medium containing 10% human A⁺ serum for 5 min at 37° C. After incubation for 15, 30 or 45 minutes the cells were washed with PBS and fixed with 2% PFA for 10 min on ice, and thereafter stained with rhodamine phalloidin or SiR-actin. Confocal images were captured using an apochromat 63×/1.4 CS2 oil-immersion objective on a Leica TCS SP8 (Leica Microsystems). The 488 nm, 561 nm and 633 nm white laser lines were used for detection and three-dimensional data was acquired from 12-bit raw imaging data used to the build Z-stacks for the individual channels by LAS X software. A spot detection mode in the Bitplan Imaris software was used to define individual phagosomes and cells were counted manually. The data is presented as the average number of phagocytosed bacteria per cell. Fluorescence voxel intensity of TRAM^{cherry} at the phagosome is also presented. Statistical significance was calculated in GraphPad Prism by One-way ANOVA Kruskal-Wallis multiple comparison test.

LDH Cytotoxicity Assay

To address the potential cytotoxicity of different peptides, supernatants from the cell-based assays were collected and analysed using the Thermo Scientific™ Pierce™ LDH Cytotoxicity Assay Kit as suggested by the manufacturer. For every assay, separate wells were seeded for the required controls (Spontaneous LDH Release Controls and Maximum LDH Release Control) with the same amount of cells as used for peptide treatments. All supernatants were analysed in triplicate with the average value of technical replicates taken into calculations.

qPCR

Total RNA was isolated from the cells using Qiazol reagent #79306 from QIAGEN, and chloroform extraction followed by purification on RNeasy Mini columns with DNAse digestion (Qiagen). cDNA was prepared using the Maxima First Strand cDNA Synthesis Kit for quantitative real-time polymerase chain reaction (RT-qPCR) (Thermo Fisher Scientific) according to the manufacturer's protocol. qPCR was performed using the PerfeCTa qPCR FastMix (Quanta Biosciences) in replicates and cycled in a StepOnePlus™ Real-Time PCR cycler. The following TaqMan® Gene Expression Assays (Applied Biosystems®) were used: IFNβ (Hs01077958_s1), TNF (Hs00174128_m1), TBP (Hs00427620_m1), CXCL10 (Hs01124251_g1), IL-6 (Hs00174131_m1) and IL-1β (Hs01555410_m1), for human cells; Ifnβ (Mm00439552_s1), Tnf (Mm00443258_m1), Tbp (Mm01277042_m1) and II-1β (Mm00434228_m1), for mouse cells. No RT controls were negative. The level of TBP mRNA was used for normalisation and results presented as relative expression compared to the control untreated sample. Relative expression was calculated using Pfaffl's mathematical model (Pfaffl, Nucleic Acids Research 29(9): e45, 2001). Results are presented as the mean and SD expression fold change for biological replicates relative to non-stimulated cells. Statistical significance was evaluated in GraphPad Prizm 5.03. Data distribution was assumed to be normal but this was not formally tested. The difference between the two groups was determined by two-tailed t test.

Expression Vectors and DNA Transfection

SLAMF1 and mutants thereof in a C-terminal Flag-tag vector and TRAM^{FLAG} construct are described in Yurchenko et al. (supra), and SLAMF1 mutants not disclosed in Yurchenko et al. were synthesised equivalently to those previously detailed; Human TRAM^{YFP}, Mal and Mal^{FLAG} were obtained from K. Fitzgerald (University of Massachusetts Medical School, Worcester, Mass., USA). IRAK1 and IRAK4^{EGFP} were obtained from MRC PPU Reagents and Services, University of Dundee, UK. HEK293T cells in 6-well plates were transfected with 0.2-0.4 μg vectors/well using Genejuice transfection reagent (Millipore). The total amount of DNA per well was always normalised to empty vectors. Media was changed 24 h after transfection and lysates were prepared 48 h after transfection.

Immunoprecipitations (IPs)

HEK293T cells expressing Flag-tagged proteins were lysed using 1×lysis buffer (150 mM NaCl, 50 mM TrisHCl (pH 8.0), 1 mM EDTA, 1% NP40), supplemented with EDTA-free Complete Mini protease Inhibitor Cocktail Tablets and PhosSTOP phosphatase inhibitor cocktail from Roche, 50 mM NaF and 2 mM Na₃VO₃ (Sigma). IPs were carried out by rotation of cell lysates at 4° C. for 2 h with anti-flag (M2) agarose (Sigma). Beads were washed 5 times with lysis buffer and heated for 5 min with 1×NuPAGE® LDS Sample Buffer (Thermo Fisher Scientific) before analysis of precipitates by Western blotting. For testing peptides as CFP-tagged constructs, primers coding for the described amino acid sequences were cloned into the ECFP-C1 vector yielding N-terminal CFP-tagged peptides.

Immunoprecipitations with the biotinylated peptides Penetratin-biotin and Pep7-Penetratin-biotin (synthesised by custom order by GenScript) were performed by coating NeutrAvidin® Agarose Resin (Thermo Scientific) with peptide by incubating 40 μl 50% agarose slurry with 10 μl 2 mM peptides in 300 μl lysis buffer (for one precipitation reaction) (300 mM NaCl, 50 mM TrisHCl (pH 8.0), 1 mM EDTA, 1% Triton X100). Beads were washed 3 times with lysis buffer to remove unbound peptides before applying cellular extracts. Cell lysis was performed in the same lysis buffer, both for HEK293T cells and primary human macrophages. Precipitations were carried out by rotation at 4° C. for 30′-1 h, followed by four washes with lysis buffer. Agarose resin with precipitated protein complexes was heated in loading buffer 1×NuPAGE® LDS Sample Buffer (Thermo Fisher Scientific) containing 20 mM DTT and analysed by Western blotting.

Western Blotting

Cell lysates, other than when used as controls in immunoprecipitations, were prepared by simultaneous extraction of proteins and total RNA using Qiazol reagent (Qiagen) as suggested by the manufacturer. Protein pellets were dissolved by heating protein pellets for 10 min at 95° C. in buffer containing 4 M urea, 1 SDS (Sigma) and NuPAGE® LDS Sample Buffer (4×) (Thermo Fisher Scientific). Otherwise, lysates were made using 1× RIPA lysis buffer (150 mM NaCl, 50 mM TrisHCl (pH 7.5), 1% Triton X100, 5 mM EDTA, protease inhibitors, phosphatase inhibitors). For Western blot analysis we used pre-cast protein gels NuPAGE™ Novex™ and iBlot Transfer Stacks iBlot Gel Transfer Device (Thermo Fisher Scientific). Proteins were transferred to the membrane by dry-blotting using iBlot® 2 NC Regular/Mini Stacks (Life Technologies) in the iBlot 2 Dry Blotting System (Life Technologies). After secondary antibody incubation, membranes were washed and incubated with HRP substrate solution (SuperSignal™ West Femto Maximum Sensitivity Substrate (Thermo Fisher Scientific)). Images were taken using Odyssey® Fc (LI-CORE). Quantification graphs were generated by the analysis of signal intensities for protein bands (protein of interest and endogenous loading control—β-tubulin from the same membrane) using Odyssey software (LI-CORE). Data were normalised to the expression levels of β-tubulin and presented as a relative fold change to the control sample.

ELISA and Multiplex Cytokine Assay

TNF in supernatants was detected using a human TNF-alpha DuoSet ELISA (DY210) (R&D Systems); CXCL10 using a DuoSet ELISA (DY266) (R&D Systems); IL-1β using a BD OptEiA human IL-1β ELISA SetII (#557953, BD Biosciences): IFNβ using a VeriKine-HS™ Human Interferon-Beta Serum ELISA Kit (#41415) from PBL Assay Science. Some supernatants were also analysed by multiplex cytokine assay (Bio-Plex; Bio-Rad Laboratories Inc.) for IL-1β, IL-6, IL-8, CXCL10/IP-10. Results are presented as mean and SD for biological replicates for a representative donor (primary human macrophages), or at least three independent experiments for the model cell line THP-1. Statistical significance was evaluated in GraphPad Prizm 5.03. Data distribution was assumed to be normal but this was not formally tested. The difference between the two groups was determined by two-tailed t test.

### Example 1—Effect of Peptide Size on Efficacy

As detailed in Yurchenko et al., murine SLAMF1 and TRAM do not interact with each other. The C-termini of human and murine SLAMF1 are largely conserved, with the exception of four amino acids at positions 329-332 of murine SLAMF1, which correspond to positions 321-324 of human SLAMF1. These amino acids have the sequence Thr-Asn-Ser-Ile in human SLAMF1 and Pro-Asn-Pro-Thr in murine SLAMF1. It was hypothesised that this sequence could be important in binding to TRAM, though it was also considered possible that this sequence could be important for the interaction in the context of the whole protein secondary structures (due to the positioning of the β-sheet formed by this sequence), but not critical for designing of small free peptides. The two proline residues in murine SLAMF1ct will change the positioning of the C-terminal β-sheet (because prolines introduce turns to the protein backbone structure), but may not have an essential role when the interaction region is freely exposed in a short peptide sequence. Peptides were designed which encompass positions 321-324 of human SLAMF1 and the surrounding regions.

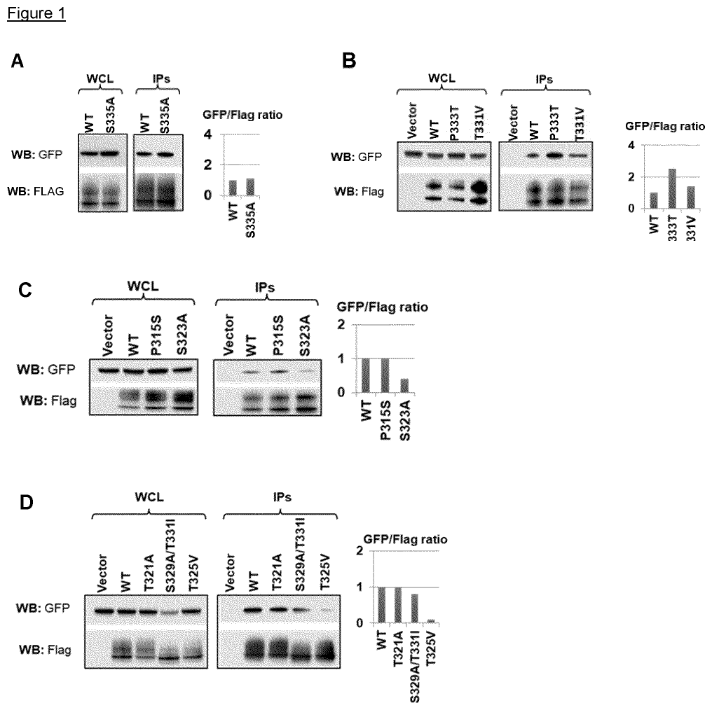

Prior to designing the peptides, we performed an immunoprecipitation screen for some of the SLAMF1 c-terminus (SLAMF1ct) variants with single or double aa substitutions to identify amino acid residues important in binding of SLAMF1 to TRAM (FIG. 1). It was known from Yurchenko et al. (supra) that tyrosine 327 could be substituted to phenylalanine. It was also found that the S335A substitution has no effect on the interaction (FIG. 1A) and that the P333T substitution, which is a natural SLAMF1 variant (SNP), strongly enhances the SLAMF1-TRAM interaction (FIG. 1B). The T331V substitution was also found to have no effect on the interaction (FIG. 1B), as was the P315S substitution (FIG. 1C) and the T321A substitution (FIG. 1D). The double substitution S329A/T331I also did not significantly affect the interaction (FIG. 1D). The S323A (FIG. 1C) and T325V (FIG. 1D) substitutions were found to decrease the interaction between TRAM and SLAMF1.

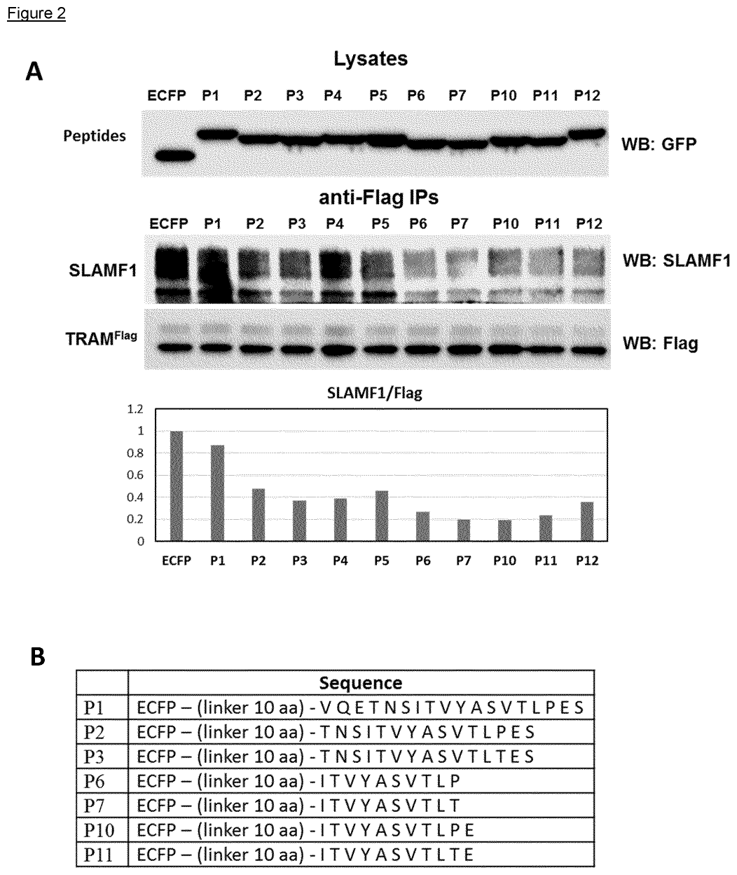

Inhibition of the TRAM-SLAMF1 interaction using SLAMF1-derived peptides was tested. For the first line of experiments, SLAMF1-derived peptides were co-expressed as N-terminal ECFP-tagged proteins together with SLAMF1 and TRAM^{FLAG} constructs in HEK-293T cells. Cell lysates were used for anti-Flag immunoprecipitations (IPs) as shown in FIG. 2A, demonstrating an inhibitory effect of a number of ECFP-tagged peptides (FIG. 2B) on SLAMF1-TRAM co-precipitation (linker sequence is set forth in SEQ ID NO: 99, derived from the commercial ECFP-C1 plasmid). The shorter sequences P6 (SEQ ID NO: 2) and P7 (SEQ ID NO: 1) demonstrated the highest efficacy in these assays. Of these two peptides, in silico analysis predicted that the P7 peptide would have higher solubility than the P6 peptide (not shown).

Modelling of peptide secondary structures with an attached Arg11 CPP (SEQ ID NO: 48) was performed using the PEP FOLD server (http://bioservspbs.univ-paris-diderotfr/services/PEP-FOLD/). This showed more potential intramolecular interactions between P6 and the Arg11 CPP than between P7 and Arg11 (not shown). Such internal contacts could interfere with cell penetration or the affinity of the peptide for the target protein. Modelling of a longer peptide (P3, SEQ ID NO: 49) attached to the Arg11 CPP showed an even higher probability of formation of a closed structure with electrostatic contacts between P3 and the Arg11 CPP (not shown). Accordingly a longer peptide may not penetrate effectively, or form a secondary structure with incorrect positioning of the TRAM-interacting motif interfering with the affinity of the peptide for TRAM. Accordingly, peptide P7 was selected as lead candidate of the tested candidate sequences.

### Example 2—Intracellular Localization of P7-Arg11

Before proceeding with functional analysis, we tested the penetrating ability of CPP-tagged P7, and its resulting intracellular distribution in human primary macrophages. P7 peptide tagged with Arg11 CPP was labeled with TAMRA fluorescent dye (TAMRA-P7-Arg11) along with Arg11 alone (TAMRA-Arg11) and applied to live human macrophages differentiated from PBMCs to follow intracellular distribution of the peptides. P7-Arg11 (SEQ ID NO: 51) was distributed both to vesicles and cytoplasm in the cells (not shown) showing potential for co-localization with cytoplasmic targets.

### Example 3—Primary Functional Testing of P7 Peptide

We tested whether pre-treatment of THP-1 macrophages with the P7-Arg11 peptide would have an inhibitory effect on LPS-mediated IFN/3, TNF and CXCL10 mRNA expression.

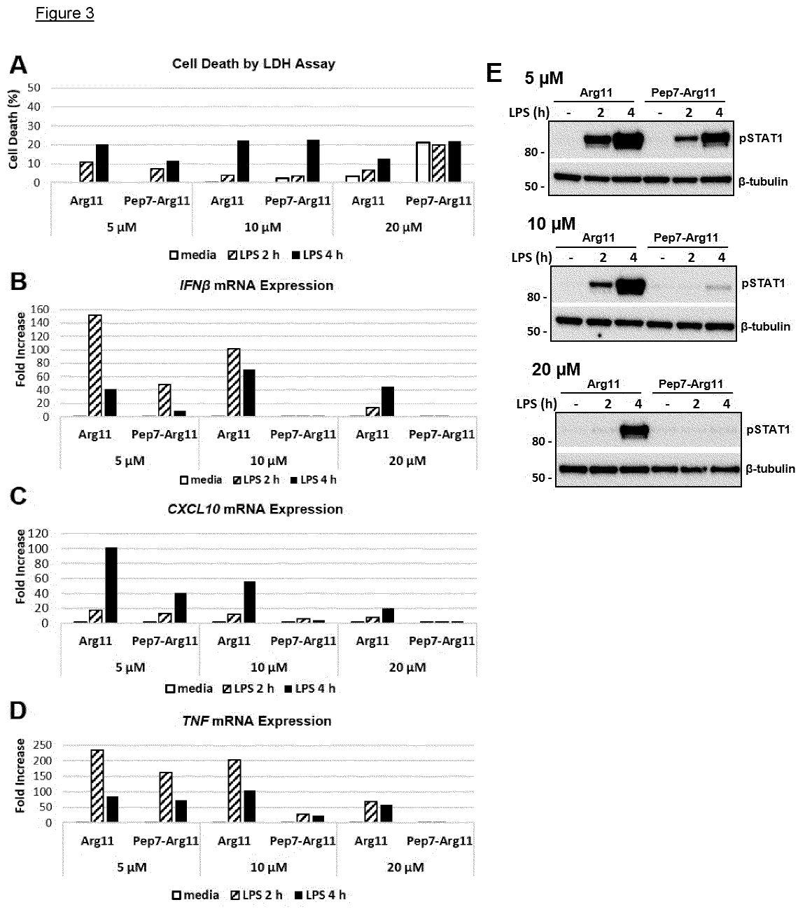

THP-1 cells were pre-treated with increasing concentrations of P7-Arg11 and control peptide Arg11 for 1 h (FIG. 3). FIG. 3A shows the results of an LDH assay, demonstrating that concentrations of 5 μM and 10 μM P7-Arg11 were essentially non-toxic, but that increased toxicity was seen at a concentration of 20 μM. At a concentration of 10 μM, P7-Arg11 displayed a clear inhibitory effect on LPS-mediated IFNβ, CXCL10 and TNF expression, and at a P7-Arg11 concentration of 20 μM expression of IFNβ, CXCL10 and TNF mRNA was totally inhibited (FIG. 3B-D). Phosphorylation of STAT1 is initiated by binding of secreted IFNβ to the IFN receptor (IFNAR). In line with the cytokine expression data, phosphorylation of STAT1 was strongly inhibited by P7-Arg11 at 10 μM concentration, and fully abrogated by 20 μM P7-Arg11 (FIG. 3E). Inhibition of LPS-mediated TNF production was confirmed by ELISA, demonstrating that downregulation of TNF expression by P7-Arg11 also prevented TNF secretion (not shown).

In further experiments peptides were removed from media after pre-treatment of macrophages but before LPS stimulation. No changes in LPS-induced cytokine expression levels (IFNβ, TNF and CXCL10 mRNA expression) were observed for P7-Arg11 pre-treated cells, which demonstrates a reversible effect of the P7-Arg11 peptides (data not shown).

Due to the larger molecular weight of P7-Arg11 than of the Arg11 CPP alone, we required another more suitable control peptide with a similar molecular weight and amino acid composition to P7. We tested the control/decoy peptide P7C3 (SEQ ID NO: 50), which contains 4 amino acid substitutions relative to Pep7 but has a similar molecular weight to P7-Arg11, the same length as P7-Arg11 and an almost identical overall amino acid composition to P7-Arg11. Pre-treatment of THP-1 cells with P703-Arg11 was found to have no effect on LPS- or E. coli particle-induced expression of IFNβ, CXCL10 and TNF (data not shown).

### Example 4—CPP Screening

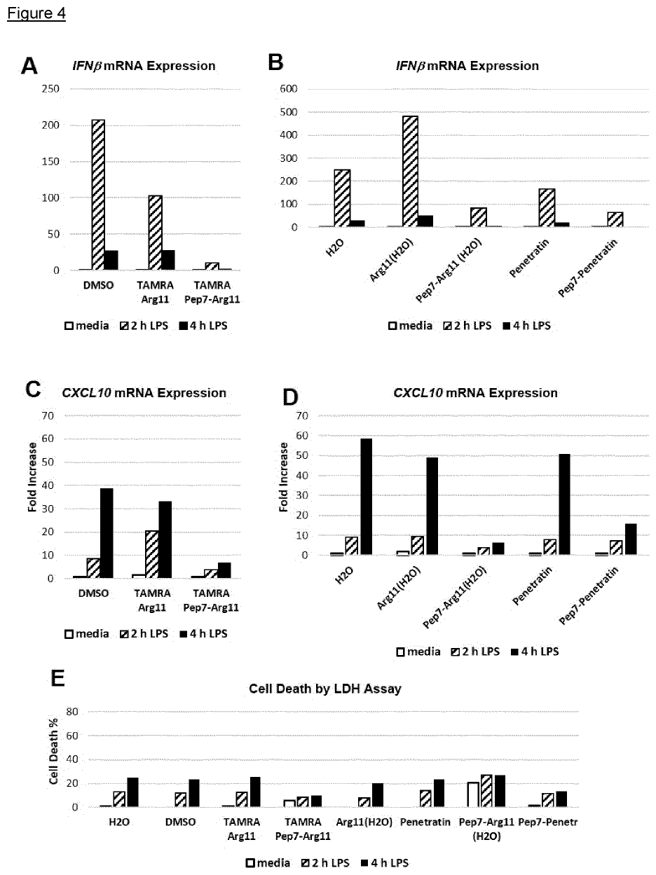

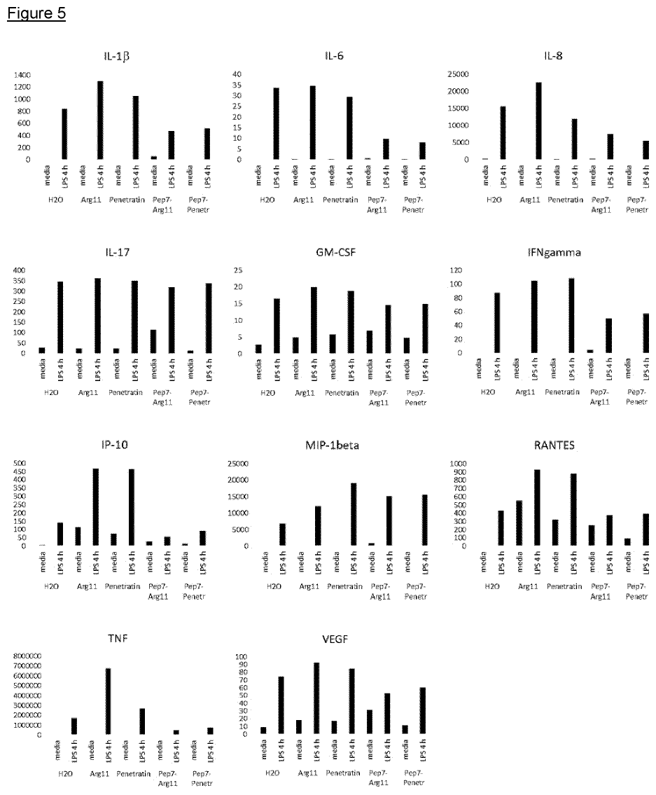

P7/Pep7 linked to two different CPPs—Arg11 and penetratin (Pen, SEQ ID NO: 8) with or without a TAMRA label was equally effective in down-regulating LPS-mediated expression of IFNβ (FIG. 4A-B) and CXCL10 (FIG. 4C-D) mRNA. P7-Pen (SEQ ID NO: 10) showed no background toxicity when compared to TAMRA-P7-Arg11 or water-diluted P7-Arg11 (FIG. 4E). Supernatants from these experiments were analysed by 27-plex Bioplex assay to address the potential impact of the P7 peptide on LPS-mediated secretion of a panel of cytokines (FIG. 5). Only those cytokines from the 27-plex panel which were detectable in THP-1 cell supernatants after LPS stimulation are included in the figure. Both peptides P7-Pen and P7-Arg11 demonstrated an inhibitory effect on the LPS-mediated secretion of IL-18, IL-6, IL-8 and TNF by THP-1 macrophages.

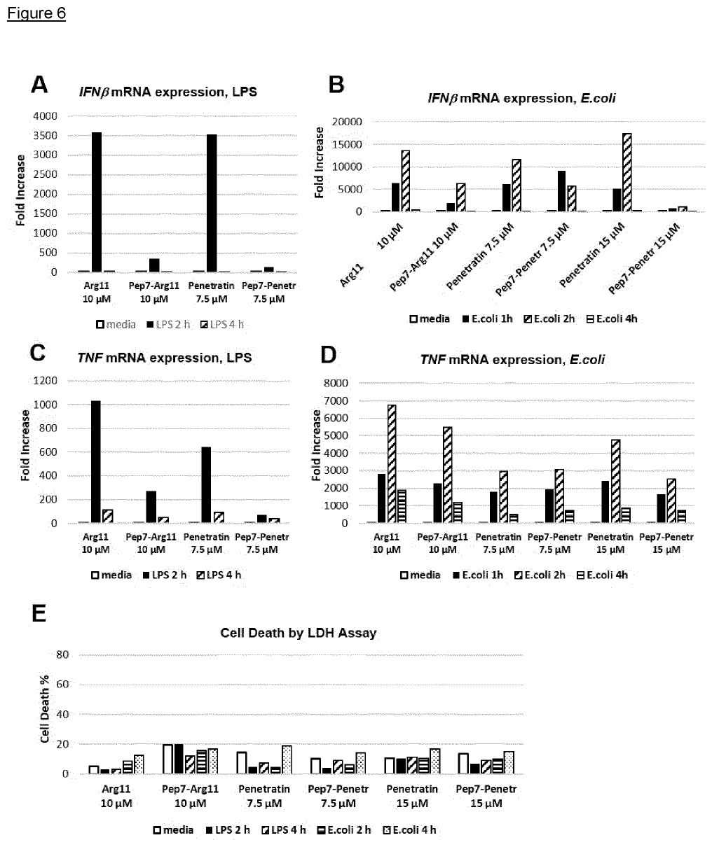

In light of these results in the THP-1 model system, we proceeded with testing P7 peptides linked to two different CPPs (and at two different concentrations) in PBMC-derived human primary monocytes stimulated by LPS or E. coli particles (FIG. 6). In the pilot test we observed inhibition of IFN/3 and TNF mRNA expression by both peptides, especially in LPS-stimulated cells (FIG. 6A-B). P7-Pen (15 μM) was more effective than P7-Arg11 (10 μM) in reducing cytokine secretion by monocytes stimulated by E. coli particles (FIG. 6B-D). Again, P7-Arg11 demonstrated higher basal toxicity when compared to control peptides or P7-Pen (FIG. 6E).

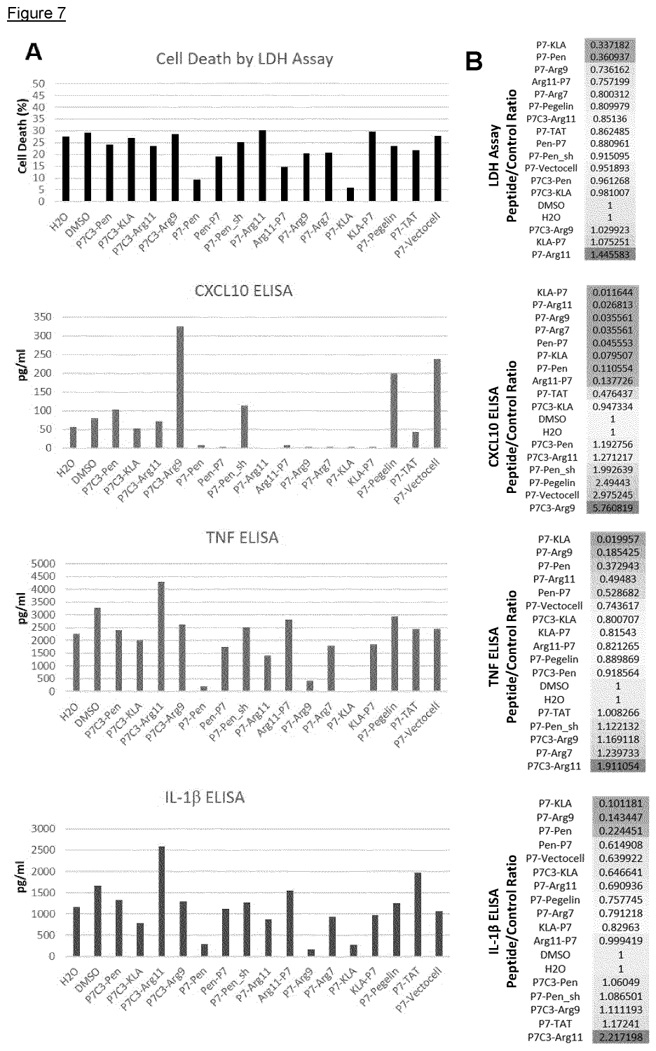

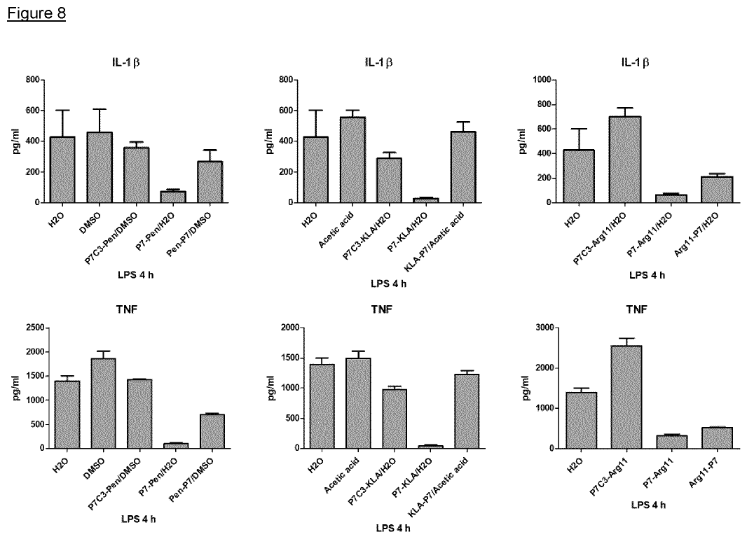

We then set up a CPP screen in THP-1 cells to identify the best CPPs for P7 delivery (FIG. 7). Sequences of peptides used in the screen are listed in Table 2.

TABLE 2

| | | SEQ |
|---|---|---|
| | | ID |
| Name | Sequence | NO |
| P7- | ITVYASVTLTGRRRRRRRRRRR | 51 |
| Arg11 | | |
| Arg11- | RRRRRRRRRRRGITVYASVTLT | 52 |
| P7 | | |
| P7C3- | IATYASTALTGRRRRRRRRRRR | 53 |
| Arg11 | | |
| P7-Arg9 | ITVYASVTLTGRRRRRRRRR | 13 |
| P7C3- | IATYASTALTGRRRRRRRRR | 54 |
| Arg9 | | |
| P7-Arg7 | ITVYASVTLTGRRRRRRR | 55 |
| P7-Pen | ITVYASVTLTGRQIKIWFQNR | 10 |
| | RMKWKK | |
| Pen-P7 | RQIKIWFQNRRMKWKKGITVY | 56 |
| | ASVTLT | |
| P7C3- | IATYASTALTGRQIKIWFQNR | 57 |
| Pen | RMKWKK | |
| P7- | ITVYASVTLTGRGGRLSYSRR | 58 |
| Pegelin | RFSTSTGR | |
| P7-TAT | ITVYASVTLTGGRKKRRQRRR | 59 |
| | PPQ | |
| P7-KLA | ITVYASVTLTGKLALKLALKA | 12 |
| | LKAALKLA | |
| KLA-P7 | KLALKLALKALKAALKLAGIT | 60 |
| | VYASVTLT | |
| P7C3-KLA | IATYASTALTGKLALKLALKA | 61 |
| | ALLKAKLA | |
| P7- | ITVYASVTLTGVKRGLKLRHV | 62 |
| Vectocell | RPRVTRMDV | |
| P7-Pen_sh | ITVYASVTLTGNRRMKWKK | 63 |

Several CPPs were tested in parallel—protegrin class CPPs (TAT, SEQ ID NO: 36; pegelin, SEQ ID NO: 36), amphipathic CPPs (vectocell, SEQ ID NO:43; KLA/MAP, SEQ ID NO: 9), shorter versions of Arg sequences (Arg9, SEQ ID NO: 7; Arg7, SEQ ID NO: 47), short penetratin CPP (Pen_sh, SEQ ID NO: 22) along with previously tested Pen, Arg11, some control peptides and respective solvents. Also we performed some tests to address the effect of CPP positioning (C-terminal vs. N-terminal to P7) on the inhibitory activity of P7 peptide towards expression of several LPS-mediated cytokines (CXCL10, TNF, IL-13) (FIGS. 7 & 8).

Data is presented for cytokine secretion levels in supernatants of treated cells (representative experiment, FIG. 7A). Data from several identical screens is also presented as the average value for the ratio between the levels of cytokines after pre-treatment with peptides vs. respective solvent (FIG. 7B). Ratios are presented as heat maps. As can be seen from FIG. 7, P7-KLA (SEQ ID NO: 12) and P7-Pen were most effective in reducing LPS-mediated toxicity (LDH assay), while P7-Arg9 (SEQ ID NO: 13, Arg11-P7 (SEQ ID NO: 52) and P7-Arg7 (SEQ ID NO: 55) also showed some decrease in LPS-mediated toxicity when compared to control peptide P7C3. IFNβ-induced CXCL10 secretion was strongly downregulated by P7 with N- and C-terminal Pen, N- and C-terminal Arg11, N- and C-terminal KLA, and slightly decreased by C-terminal TAT. As to the pro-inflammatory cytokines, TNF and IL-13, fewer peptides were able to inhibit their secretion: P7 with C-terminal Pen, C-terminal Arg9 and C-terminal KLA CPPs (FIGS. 7 & 8). Thus, CPP positioning was critical for inhibition of LPS-mediated pro-inflammatory cytokines, but not for inhibition of LPS-induced IFNβ-mediated CXCL10 secretion (reflecting IFNβ secretion), in that this was achieved only by peptides with the CPP at the C-terminus.

### Example 5—Screening Longer SLAMF1-Derived Peptides

In the next screens we tested if the extension of peptides at the N- or C-terminus could alter their efficacy, and peptides used for the screens are listed in Table 3.

TABLE 3

| | | SEQ |
|---|---|---|
| | | ID |
| Name | Sequence | NO |
| P7-Pen | ITVYASVTLTGRQIKIWFQNRRMKWKK | 11 |
| P6-Pen | ITVYASVTLPGRQIKIWFQNRRMKWKK | 12 |
| P3-Arg11 | TNSITVYASVTLTESGRRRRRRRRRRR | 64 |
| P3sh1-Pen | TNSITVYASVTLTGRQIKIWFQNRRMK | 65 |
| | WKK | |
| P3sh2-Pen | NSITVYASVTLTGRQIKIWFQNRRMKW | 66 |
| | KK | |
| P10-Pen | ITVYASVTLPEGRQIKIWFQNRRMKWKK | 67 |
| P10-Ala11- | ITVYASVTLPAGRQIKIWFQNRRMKWKK | 68 |
| Pen | | |
| P11-Pen | ITVYASVTLTEGRQIKIWFQNRRMKWKK | 69 |
| P11-Ala11- | ITVYASVTLTAGRQIKIWFQNRRMKWKK | 70 |
| Pen | | |

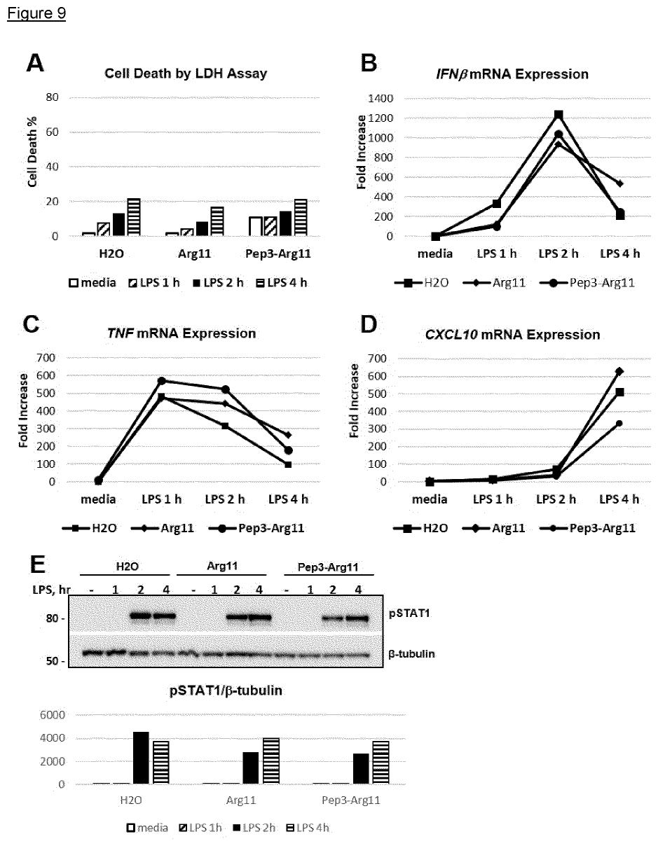

As we expected from the peptide modelling (see above), the P3-Arg11 peptide (SEQ ID NO: 64) was not effective in reducing LPS-mediated cytokine mRNA expression (FIG. 9). P3 is longer than P7 by 3 amino acids at the N-terminus and 2 amino acids at the C-terminus. Western blot analysis of THP-1 cells pre-treated with P3-Arg11 revealed no effect of this peptide on the phosphorylation of STAT1 upon LPS stimulation when compared to the control peptide (FIG. 9E).

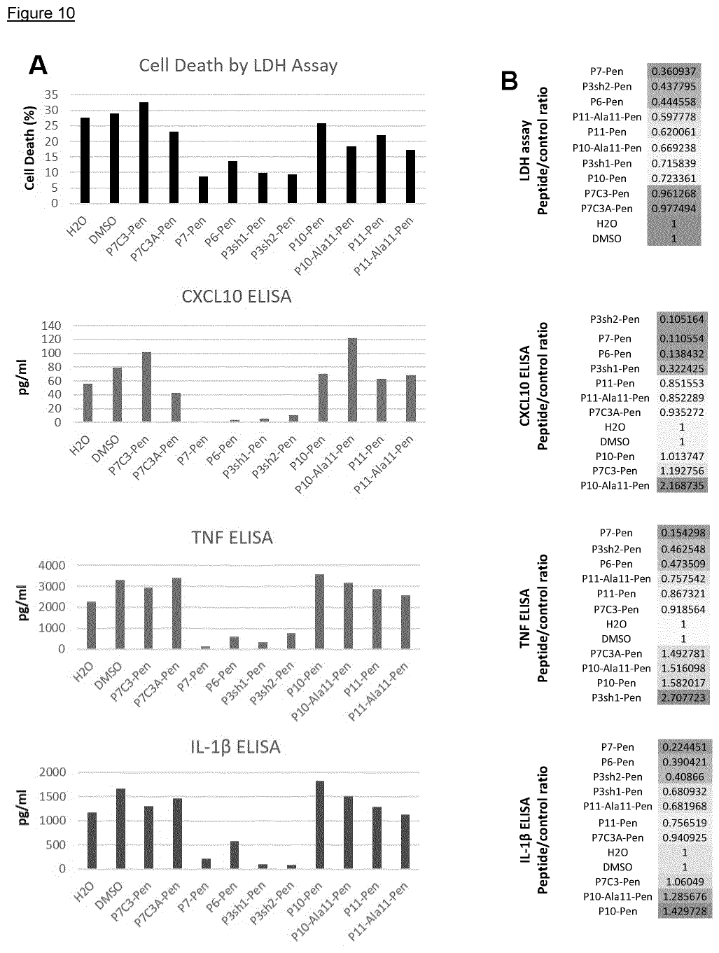

To test which additional amino acids are responsible for the loss of the peptide's inhibitory effect, THP-1 cells were pre-treated with several variants of SLAMF1-derived peptides, which corresponded to P7 elongated at either the N-terminus or the C-terminus (FIG. 10). These screens demonstrated that extension of the P7 peptide at the N-terminus by up to 3 amino acids (see P3sh1 (extended by 3 amino acids at N-terminus relative to P7) and P3sh2 (extended by 2 amino acids at N-terminus relative to P7)) did not cause loss of inhibitory activity towards expression of TLR4-induced proinflammatory cytokines or IFNβ-dependent CXCL10 secretion (FIG. 10). However, extension of peptide P6 or P7 at the C-terminus by even 1 amino acid, whether with a negatively charged Glu residue (P10 (SEQ ID NO: 71) and P11 (SEQ ID NO: 72)) or an amino acid with a hydrophobic side chain, i.e. an Ala residue (P10-Ala11 (SEQ ID NO: 73) or P11-Ala11 (SEQ ID NO: 74)) totally abrogated the peptide's inhibitory activity (FIG. 10).

### Example 6—Screen of Substitutions within P7

The sequences of peptides synthesised for the screen of single amino acid substitutions in P7 are listed in Table 4.

TABLE 4

| | | SEQ |
|---|---|---|
| | | ID |
| Name | Sequence | NO |
| P7-Pen | ITVYASVTLTGRQIKIWFQNRRMKWKK | 10 |
| P7-A1-Pen | ATVYASVTLTGRQIKIWFQNRRMKWKK | 75 |
| P7-A2-Pen | IAVYASVTLTGRQIKIWFQNRRMKWKK | 76 |
| P7-S2-Pen | ISVYASVTLTGRQIKIWFQNRRMKWKK | 77 |
| P7-N2-Pen | INVYASVTLTGRQIKIWFQNRRMKWKK | 15 |
| P7-A3-Pen | ITAYASVTLTGRQIKIWFQNRRMKWKK | 78 |
| P7-T3-Pen | ITTYASVTLTGRQIKIWFQNRRMKWKK | 79 |
| P7-L3-Pen | ITLYASVTLTGRQIKIWFQNRRMKWKK | 80 |
| P7-A4-Pen | ITVAASVTLTGRQIKIWFQNRRMKWKK | 14 |
| P7-V4-Pen | ITVVASVTLTGRQIKIWFQNRRMKWKK | 81 |
| P7-T4-Pen | ITVTASVTLTGRQIKIWFQNRRMKWKK | 82 |
| P7-L5-Pen | ITVYLSVTLTGRQIKIWFQNRRMKWKK | 83 |
| P7-A6-Pen | ITVYAAVTLTGRQIKIWFQNRRMKWKK | 84 |
| P7-A7-Pen | ITVYASATLTGRQIKIWFQNRRMKWKK | 85 |
| P7-A8-Pen | ITVYASVALTGRQIKIWFQNRRMKWKK | 86 |
| P7-A9-Pen | ITVYASVTATGRQIKIWFQNRRMKWKK | 87 |
| P7-I9-Pen | ITVYASVTITGRQIKIWFQNRRMKWKK | 88 |
| P7-G9-Pen | ITVYASVTGTGRQIKIWFQNRRMKWKK | 89 |
| P7-A10-Pen | ITVYASVTAAGRQIKIWFQNRRMKWKK | 90 |
| P7-S10-Pen | ITVYASVTLSGRQIKIWFQNRRMKWKK | 91 |
| P7-V10-Pen | ITVYASVTLVGRQIKIWFQNRRMKWKK | 92 |
| P6-Pen | ITVYASVTLPGRQIKIWFQNRRMKWKK | 11 |
| P7C3A-Pen | IATYASVTLTGRQIKIWFQNRRMKWKK | 93 |

The screen was performed in THP-1 cells pre-treated with 15 μM peptides for 30 min and stimulated with LPS for 5 h. We found that the amino acids at positions 2, 4 and 10 of the P7 peptide could be substituted to other amino acids without an increase in peptide toxicity (FIG. 11, LDH assays), or loss of the peptide's inhibitory activity towards CXCL10, TNF and IL-1β secretion (FIG. 11). A shown, the substitution with the least effect on peptide activity was the Y4A substitution (P7-A4-Pen). Several other aa substitutions only partially decreased P7 peptide activity, such as V3L (P7-L3-Pen, SEQ ID NO: 80), and some amino acid substitutions totally abrogated activity, such as I1A (P7-A1-Pen, SEQ ID NO: 75) (FIG. 11).

### Example 7—Timing of Cell Treatment with Peptide

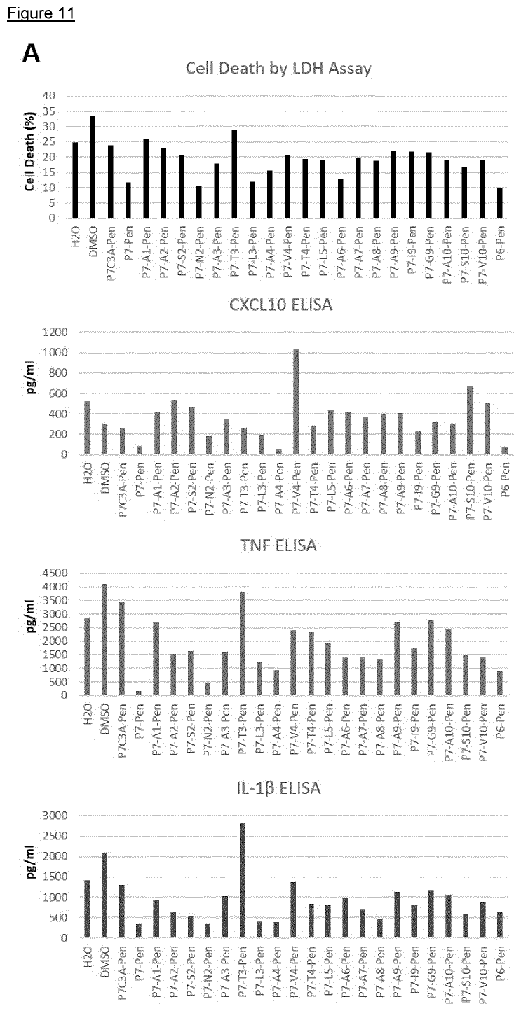

Next, we examined if the inhibitory effect of the peptide on the TLR4-IFNβ signaling axis would be preserved if the peptide was added 30 min before addition of LPS, simultaneously with LPS or 1 h after LPS treatment. We found that the peptide remained very effective in inhibition of CXCL10 secretion when applied to cells 30 min before LPS addition, and when applied simultaneously with LPS (FIG. 12). When the P7-Pen peptide was applied 1 h after addition of LPS, its inhibitory effect on CXCL10 secretion was reduced (FIG. 12), because the functional complex of TLR4 and its downstream adaptor proteins was formed, and the IFNβ signaling axis activated, before the peptide was added.

### Example 8—Effect of P7 on Murine Cells

Murine disease models are the most accessible and widely used pre-clinical model for testing new drugs. We decided to test if the P7-Pen peptide would interact with murine TRAM protein, which would suggest a similar effect of the P7 peptide on TLR4-mediated signaling in murine cells. To test the potential interaction of the P7 peptide with murine TRAM vs. human TRAM protein, we performed precipitations of C-terminally biotinylated Pen or P7-Pen peptides. Human or murine TRAM coding constructs were overexpressed in HEK 293T cells, and lysates used for precipitations on Neutravidin agarose beads. We found that P7-Pen peptide could effectively precipitate not only human TRAM, but also murine TRAM protein (data not shown).

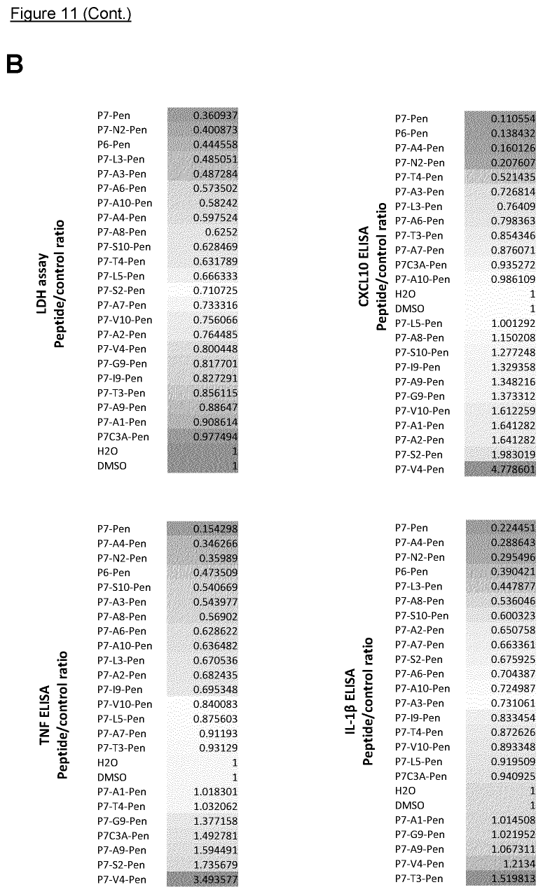

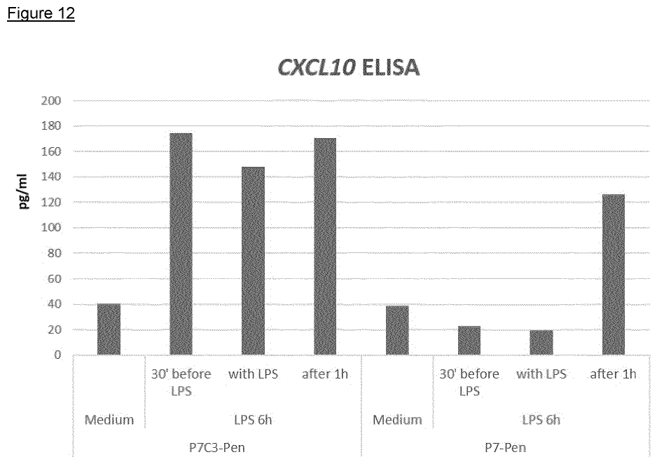

We proceeded with cellular assays to address the effect of peptide pre-treatment on TLR4-mediated cytokine expression in B6 immortalised murine macrophages (FIG. 13). Tested peptides had no cytotoxic effect on B6 murine macrophages (FIG. 13A). P7-Pen, P7-KLA and P7-A4-Pen all strongly inhibited TLR4-mediated Ifnβ, Tnf and II-1β mRNA expression by B6 cells at a concentration of 10 μM (FIG. 13B-D). CPP positioning (N- or C-terminal) had a similar effect on the ability of the P7 peptide to inhibit TLR4-mediated Ifnβ and proinflammatory Tnf and II-1β cytokine expression as found in human cells, with a more uniform effect of P7 with C-terminal positioning of Pen (FIG. 14).

### Example 9—Effect of P7 Peptide on Primary Human Monocytes and Whole Blood

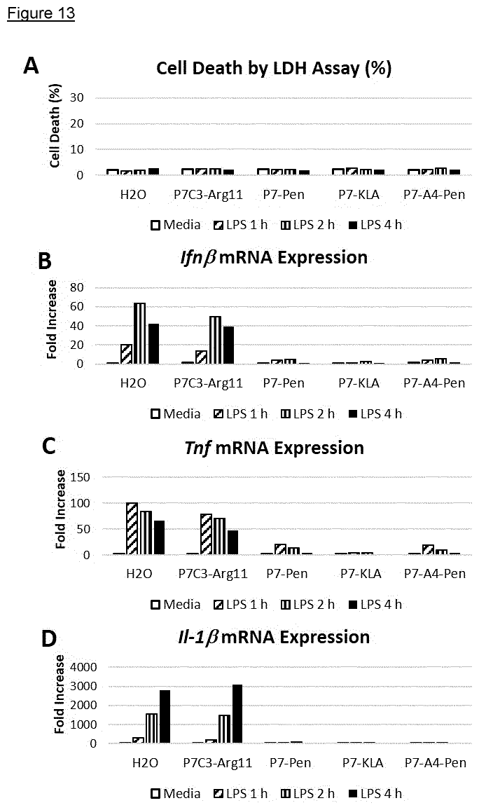

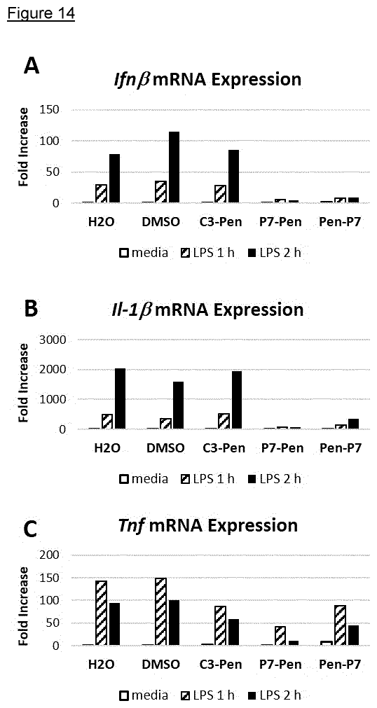

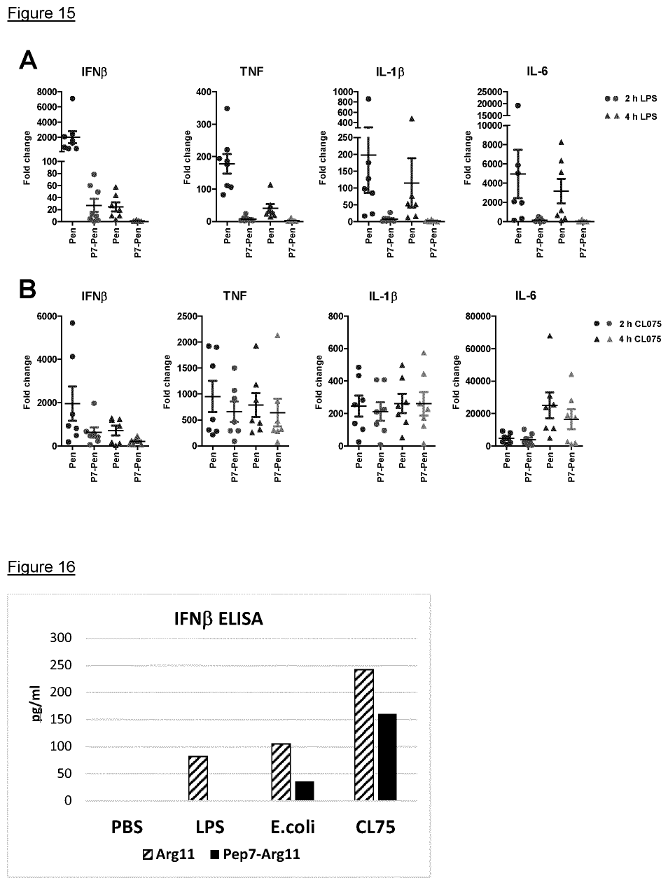

To clarify the inhibitory potential of the P7 peptide on TLR-mediated signaling in primary human cells, we examined the peptide's effect on peripheral blood (PB) monocytes stimulated by TLR4 and TLR8 ligands. As seen before in THP-1 macrophages (FIGS. 3-5) and in the pilot assay with human monocytes (FIG. 6), pre-treatment of monocytes from all tested donors with P7-Pen significantly decreased IFNβ, TNF, IL-1β and IL-6 expression for all tested donors in response to TLR4 stimulation (FIG. 15A). Unexpectedly, we found that P7-Pen also inhibited CL075-induced TLR8-mediated IFNβ mRNA expression for all tested donors, though no significant effect on the expression of proinflammatory cytokines was seen in this context (FIG. 15B). This inhibitory effect of the P7 peptide on TLR8-mediated IFNβ expression could not be explained by the inhibition of the SLAMF1-TRAM interaction and suggested that P7 has additional targets involved in TLR signalling pathways.

Next, we proceeded with a pilot whole blood assay to determine whether the P7 peptide could downregulate TLR4-mediated IFNβ secretion induced by LPS or E. coli particles. Due to the detected inhibitory activity of the P7 peptide on TLR8-mediated IFNβ expression by primary human monocytes, a TLR8 ligand was also included in the pilot whole blood assay (FIG. 16). This assay demonstrated that the P7 peptide could be used to inhibit TLR4-mediated IFNβ secretion (LPS- or E. coli-treated samples), and also has some inhibitory effect on TLR8-mediated IFNβ secretion. Without being bound by theory, inhibition of IFNβ secretion by whole blood cells in response to TLR4 and TLR8 ligands may be mainly mediated by the effect of P7 on signaling initiated by PB monocytes. TLR8 is a major sensor for Gram positive bacteria (Ehrnström et al., Frontiers in Immunology 8, Art. 1243 (2017)). Thus, inhibition of TLR8-mediated IFNβ secretion makes the P7 peptide applicable for targeted inhibition of IFNβ induced by Gram-positive species.

Therefore, CL075, LPS and Gram-negative and Gram-positive bacterial particles were included in a wider setup for whole blood assays, in which the secretion of IFNβ and a range of pro-inflammatory cytokines was monitored (FIG. 17). We found that P7-Pen effectively abrogated TLR4- and TLR8-mediated IFNβ release by whole blood cells for all tested donors. The effect of P7-Pen on LPS/TLR4-induced pro-inflammatory cytokine secretion was not as strong as the effect on IFNβ secretion, but a significant reduction in TNF, IL-6 and IL-1β secretion was nonetheless seen compared to control peptides (mean for values from Pen, P7C3-Pen or P7C3A-Pen treated samples) and IL-6, IL-1β when compared to water (solvent) control (FIG. 17A).

While significantly inhibiting E. coli-mediated IFNβ secretion, P7-Pen did not have so clear an effect on all pro-inflammatory cytokines. Significant inhibition of TNF production was seen, a degree of inhibition of IL-6 (though not to a statistically significant degree) and no significant effect on IL-1β secretion (FIG. 17C). However, this was a preliminary study and with some routine optimisation inhibition of E. coli-mediated IL-6 and IL-1β secretion is also expected, as seen with LPS.

As seen in monocytes and the pilot whole blood assay, P7-Pen inhibited TLR8-mediated IFNβ secretion but had no significant effect on pro-inflammatory cytokine secretion (FIG. 17B). In line with this, P7-Pen significantly downregulated S. aureus-mediated IFNβ secretion, which it is known to be TLR8-dependent. But in addition, P7-Pen also significantly decreased S. aureus-mediated TNF, IL-6 and IL-1β pro-inflammatory cytokine secretion. Overall, these results show promise for the use of P7-Pen in treatment of both Gram-negative and Gram-positive bacteria-induced sepsis.

### Example 10—Effect of Peptides on Signalling Via Other PRRs or the IL-1β Receptor

P7-Pen was then screened for effects on signaling from several other pattern recognition receptors (PRRs). LPS-stimulation was performed in parallel as a positive control for P7-Pen-mediated inhibition of cytokine expression.

The effect of P7-Pen on TLR2-mediated pro-inflammatory cytokine expression was investigated. Pre-treatment of primary human macrophages with P7-Pen totally abrogated TLR4-mediated expression of TNF, IL-6 and IL-1β mRNA, but had no effect on expression of pro-inflammatory cytokines induced by a TLR2/6 agonist—the synthetic lipopeptide FSL-1 (Pam₂CGDPKHPKSF, SEQ ID NO: 94) (data not shown).

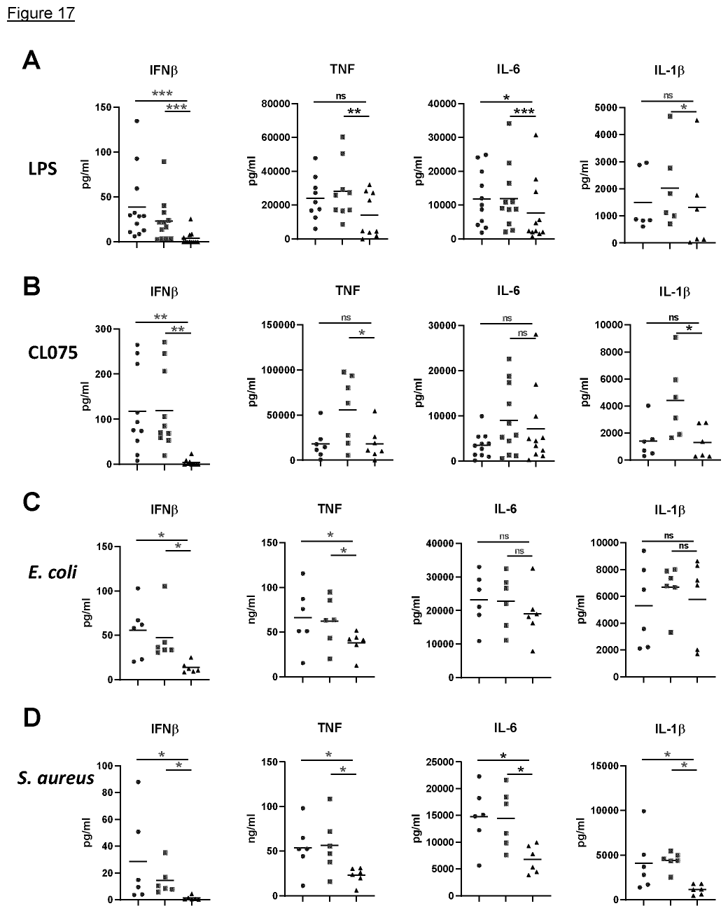

As previously shown, P7-Pen inhibits TLR8-mediated IFNβ expression in human monocytes, without significant effect on the expression of pro-inflammatory cytokines. Human cells express one more endosomal RNA sensor in addition to TLR8: TLR7, which uses similar signaling pathways to induce cytokine expression as the TLR8 receptor. To investigate the effect of P7-Pen on TLR7-mediated signaling, human monocytes were stimulated with imiquimod (R837), an imidazoquinoline amine analog to guanosine, which activates only TLR7, not TLR8 (Lee et al., PNAS 100(11): 6646-6651 (2003)). As shown in FIG. 18, P7-Pen inhibits TLR7-mediated IFNβ mRNA expression, and delayed activation of IL-6 expression (probably IFNβ-dependent). TNF and IL-1β expression were not induced in monocytes by the R837 TLR7 agonist even in control treated cells (FIG. 18C). Thus, P7-Pen has an inhibitory effect on both TLR7 and TLR8-mediated IFNβ expression in human cells.

We also tested the effect of P7-Pen on cytokine expression mediated by TLR7 in murine macrophages (using the murine TLR7 agonist resiquimod (R848) to activate TLR7), and found that the peptide had no effect on TLR7-mediated cytokine expression (data not shown). Thus, P7-Pen interferes with signaling initiated by human TLR7 and TLR8, but has no effect on signaling via murine TLR7 (murine TLR8 is believed to be non-functional). As previously shown, P7-Pen efficiently inhibits TLR4-mediated cytokine expression in both human and murine macrophages.

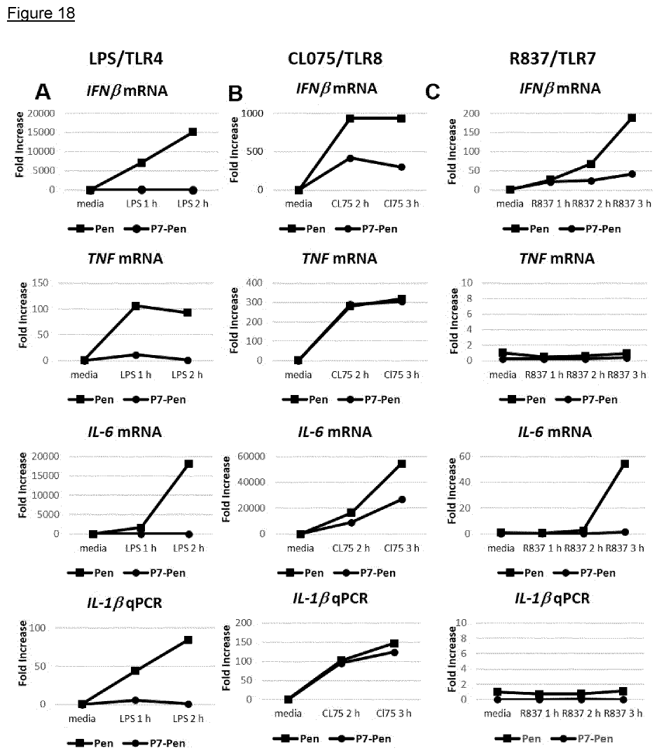

We then tested whether P7-Pen could alter signaling via another endosomal PRR: TLR9, which recognises specific unmethylated CpG motifs prevalent in microbial DNA. In humans, TLR9 is almost exclusively expressed in B cells and plasmacytoid dendritic cells (pDC), while in mice TLR9 is expressed more widely, including in myeloid immune cells. To test the effect of P7-Pen on signaling via human TLR9 we used a model system of THP-1 cells expressing an inducible TLR9^{CHERRY} construct. 48 hr after induction of TLR9 expression by doxycycline, undifferentiated THP-1 TLR9^{CHERRY} cells were pre-treated with control or P7-Pen peptide for 1 hr, then stimulated with the Class B CpG oligonucleotide CpG 2006 ODN (a human-specific TLR9 agonist). P7-Pen did not alter cell viability during stimulation of cells with CpG oligonucleotide (FIG. 19A), and strongly inhibited TLR9-mediated IFNβ and CXCL10 expression, and to some extent inhibited expression of TNF and IL-1β as well (FIG. 19B-E). However, IL-113 secretion levels were only slightly inhibited by P7-Pen (FIG. 19F). Thus, P7 peptide could also be used to inhibit the expression of selected cytokines mediated by TLR9 in human disease.

TLR2 and 4, and the endosomal TLRs 7-9, use the Myddosome signaling complex to activate transcription of pro-inflammatory cytokines, and endosomal TLRs also activate expression of type I IFNs via the same signaling complex. There are also several IL-1-like receptors including the IL-1 receptor (IL-1R) itself, which is characterized by extracellular immunoglobulin-like domains and intracellular Toll/Interleukin-1R (TIR) domains. These receptors also use the Myddosome complex to initiate downstream signaling. Therefore, we decided to test if signaling downstream of human IL-1R could be altered by the P7-Pen peptide. We used HEK-Blu IL-1R cells (Invivogen) that endogenously express the human IL-1 receptor. HEK-Blue IL-1R cells express a SEAP reporter gene under the control of the IFNβ minimal promoter fused to five NF-κB and five AP-1 binding sites. Binding of IL-1β to its receptor IL-1R on the surface of HEK-Blue IL-1R cells triggers a signaling cascade leading to the activation NF-κB and the subsequent production of SEAP.

In the assay, HEK-Blue IL-1R cells were pre-treated with control peptide or P7-Pen and stimulated with human recombinant II-1β (hII-1β) for up to 3 hr or for 24 hr. Stimulation of cells by TLR2 and 4 as well as by IL-1β would result in fast posttranslational modifications of IRAK1 in the Myddosome, and in phosphorylation of p38 MAPK. Pre-treatment of HEK-Blue IL-1R cells with P7-Pen had no effect on IL-1β-induced IRAK1 posttranslational modifications or on p-p38 MAPK levels. Moreover, IL-1β-mediated secretion of SEAP in the supernatants of cells pre-treated with the control peptide Pen was comparable with cells pre-treated with the P7-Pen peptide (data not shown). This showed that P7-Pen does not entirely block the Myddosome signaling complex, but has a specific target in this complex that is crucial for the TLR4, and one of the TLR7-9, signaling axes, but is not required for TLR2 and IL-1R-mediated signaling.

The inhibitory effect of the SLAMF1-derived peptide P7-Pen on signaling via endosomal TLRs could not be explained by its targeting of the TRAM adaptor protein, since TRAM is not involved in signal transduction from endosomal TLRs. Moreover, application of P7-Pen almost completely abrogated pro-inflammatory cytokine expression and secretion in response to LPS, a TLR4 agonist, which could not be fully explained by targeting of TRAM, since the TLR4-TRIF-TRAM signaling complex enhances only the late phase of pro-inflammatory cytokine expression.

### Example 11—Bacterial Uptake and TRAM Recruitment to Bacterial Phagosome

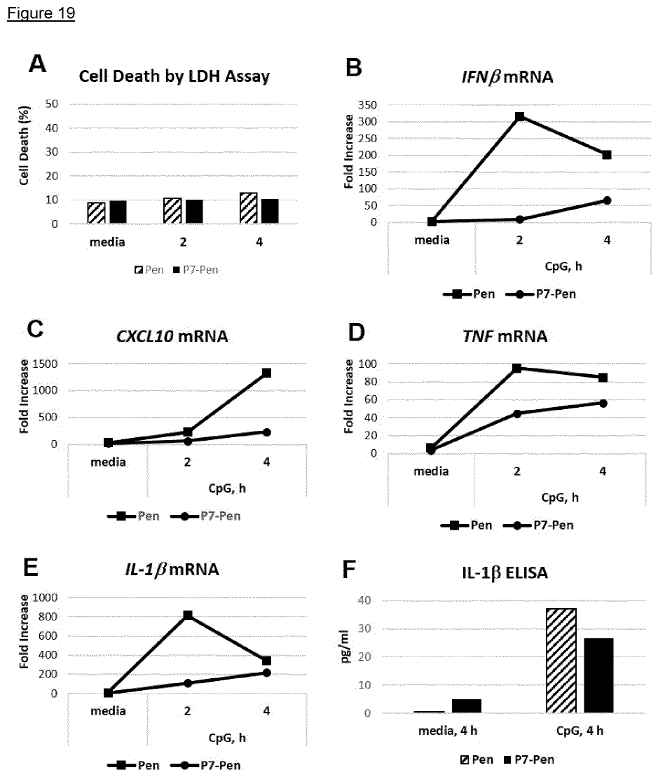

Due to the strong effect of the P7 peptide on TLR4-mediated cytokine expression and secretion, and the important role of TRAM in the regulation of bacteria phagocytosis (Skjesol et al. PLoS Pathogens 15(3): e1007684, 2019), we addressed the effect of the P7 peptide on bacteria uptake in the THP-1 model system and in primary human monocytes, and analysed the effect on TRAM recruitment to endocytosed bacteria particles in a TRAM overexpression system expressing TRAM^{CHERRY} (FIG. 20).

Cell were pre-incubated with Pen or P7-Pen peptides for 30 min, followed by the addition of Alexa Fluor 488-labeled E. coli or S. aureus bioparticles for the times indicated (FIG. 20). We found that P7-Pen peptide inhibited the uptake of E. coli particles by THP-1 WT cells and THP-1 TRAM^{CHERRY}, and of S. aureus bioparticles by THP-1 TRAM^{CHERRY} (THP-1 WT were not tested) (FIG. 20A-B), and also inhibited the uptake of E. coli particles by primary human monocytes, to a level comparable with CytoD treated cells (FIG. 20C). Overexpression of TRAM resulted in faster phagocytosis of particles, as can be seen from FIG. 20A, 15′ time point. The different kinetics seen in FIG. 20B could be due to the use of a different batch of bioparticles in this experiment.

The striking effect of the P7 peptide on bacterial uptake could explain the blockade of TLR4-mediated IFNβ secretion by this peptide, because activation of IFNβ expression can only be initiated from the endosomal compartment. This effect could also be mediated by peptide interaction with Rab11 FIP2 (data not shown), which is a crucial regulator of TRAM trafficking and the uptake of both Gram-negative and Gram-positive bacteria by macrophages (Skjesol, supra). Interestingly the peptide could also co-precipitate SLAMF1 itself (data not shown). The PHYRE2-predicted SLAMF1 cytoplasmic tail secondary structure demonstrates close intermolecular positioning of C-terminal and N-terminal β-sheets (not shown). P7 is derived from a C-terminal β-sheet, and so could complex with the SLAMF1 N-terminal beta sheet. Given the effect of P7-Pen on bacterial uptake, it could be used to block bacterial uptake by monocytes, thus making bacteria more exposed to antibiotic treatment.

### Example 12—Mechanism of Inhibition of TLR4-Mediated Cytokine Secretion by P7

It is known that regulation of cytokine expression in response to TLR ligands could differ between species, like humans and mice. In murine cells, expression of II-1β is highly dependent on the TLR4-TRAM-TRIF signaling complex (data not shown). Both Ifnβ and II-1β mRNA expression mediated by LPS or E. coli particles were totally abrogated in TIr4^{-/-} and Tram^{-/-} cells. However, Tram^{-/-} cells had intact Tnf expression in response to LPS, with a partial reduction of expression in response to bacterial stimulation (data not shown). Thus, P7-Pen's inhibitory effect on both Tnf and II-1β mRNA expression in murine cells (FIG. 13) could not be explained by targeting of TRAM.

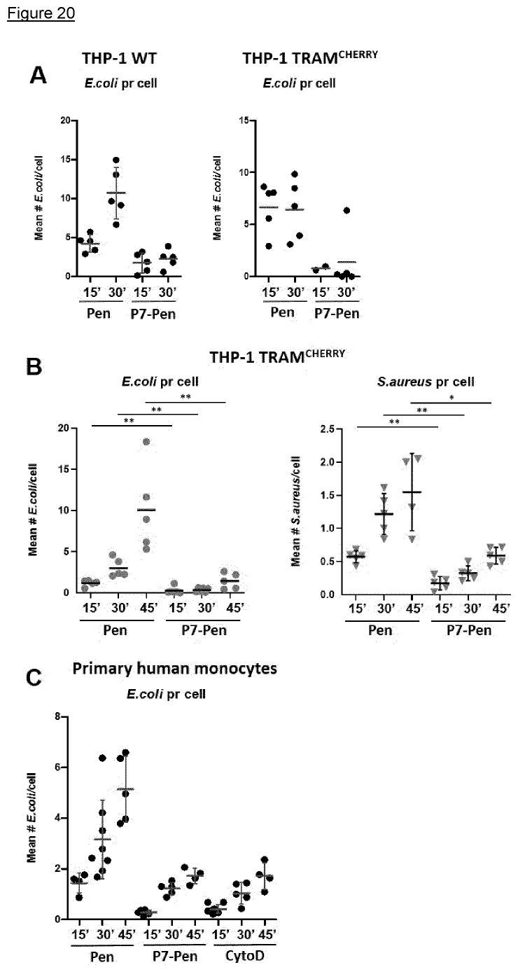

In human cells (THP-1 cell line) TRAM expression was not required for TLR4-mediated IL-1β or TNF expression in response to LPS or E. coli particles (FIG. 21, lower panels). P7-Pen was as effective in reducing expression of pro-inflammatory cytokines in THP-1 TRAM KO cells as in control cells (FIG. 22). This indicates that the P7 peptide has another target/targets crucial for regulation of pro-inflammatory cytokine expression via the Myddosome complex.

To clarify the mechanisms behind the P7-Pen inhibitory effect on the expression of TRAM-independent cytokines, we examined the effect of the peptide on posttranslational modification/phosphorylation of molecules involved in signalling through the Myddosome complex. We proceeded with precipitations of biotinylated peptides and endogenous immunoprecipitations of key signaling components of the Myddosome upon treatment of cells with control or P7 peptide.

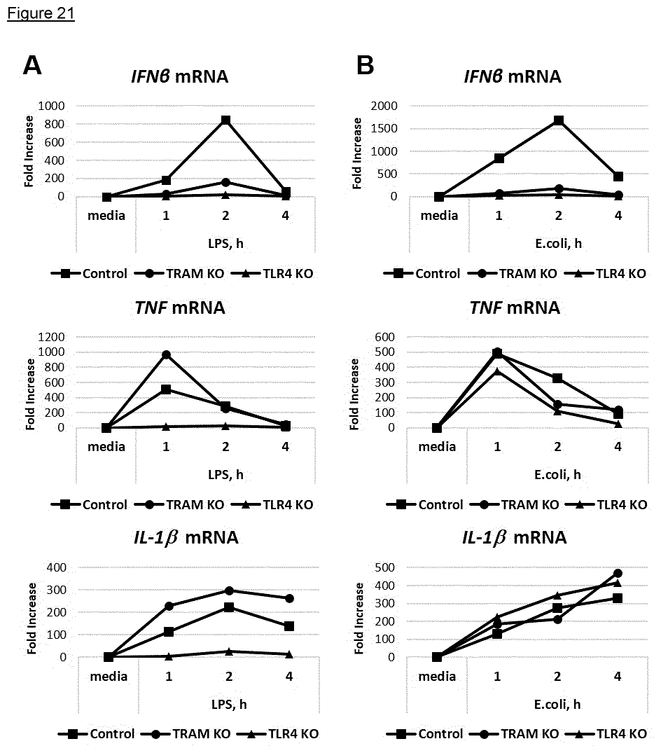

It was found that in murine macrophages the P7 peptide interfered with phosphorylation of the TAK1 kinase and IRAK1 ubiquitination/phosphorylation (modified IRAK1 is not detected with the antibody for murine IRAK1) upstream of TAK1 (FIG. 23). In addition, pre-treatment of murine macrophages with P7-Pen inhibited LPS-mediated phosphorylation of TBK1 and IκBα, and decreased phosphorylation of p38 MAPK (FIG. 23).

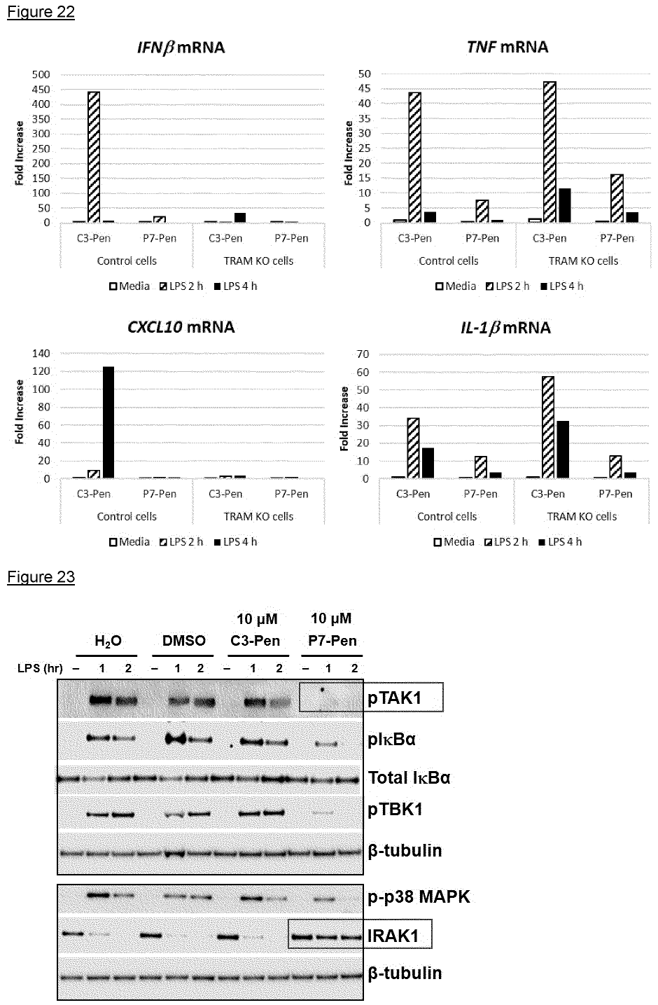

Further, we tested the effect of P7-Pen on phosphorylation of p38 MAPK, IκBα degradation and modification of IRAK1 in human macrophages (THP-1 cells) and primary human monocytes. P7-Pen inhibited LPS-mediated phosphorylation of p38 MAPK (FIG. 24A) and inhibited IRAK1 ubiquitination/phosphorylation (visualised as band shift to higher molecular weight) in primary monocytes (FIG. 24B). The same pattern was seen with cells treated with P7-A4-Pen, but not in cells treated with inactive P7-A9-Pen or control peptide P7C3-Pen (data not shown).

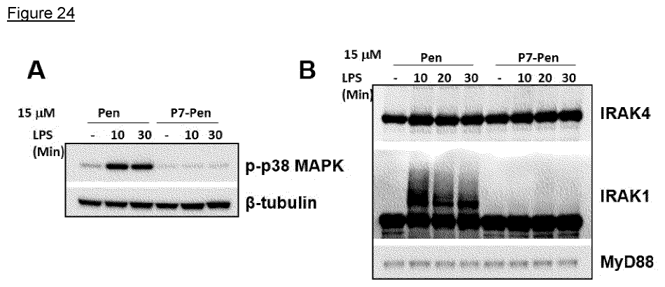

A correlation was also seen between the inhibitory effect of the P7 peptide on pro-inflammatory cytokine expression and phosphorylation/activation of p38 MAP kinase in primary human macrophages (FIG. 25). Phosphorylation of p38 MAPK and IL-6, TNF and IL-1β expression was fully inhibited by P7 only in LPS-treated cells, and not in FSL-1- or CL075-treated cells.

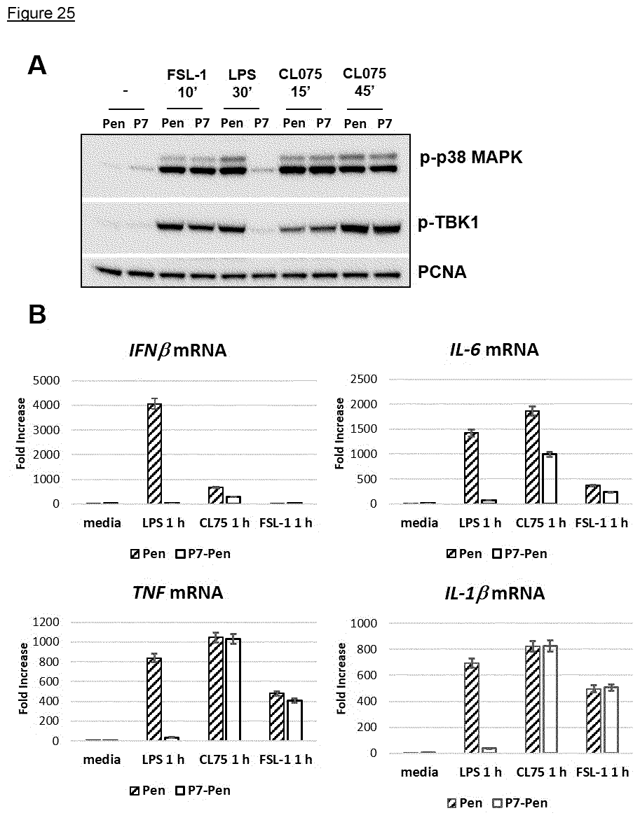

To look for interactions between P7 and proteins in the Myddosome complex, biotinylated P7 peptides were pre-incubated with lysates of primary human macrophages (unstimulated and stimulated by LPS or CL075) and NeutrAvidin beads, followed by Western blot analysis of precipitates (FIG. 26). We found that in addition to TRAM, which was very effectively precipitated by P7-Pen from the lysates when compared to the input levels, several other proteins also precipitated with P7-Pen from lysates of unstimulated cells and LPS-treated cells: MyD88, IRAK1, IRAK4 and Mal (FIG. 26). Upon stimulation with LPS, the complex of P7-interacting proteins attracted TRAF6 and TAK1 (FIG. 26A). This suggests that P7 could interact with one or more of MyD88, IRAK1, IRAK4 and Mal/TIRAP.

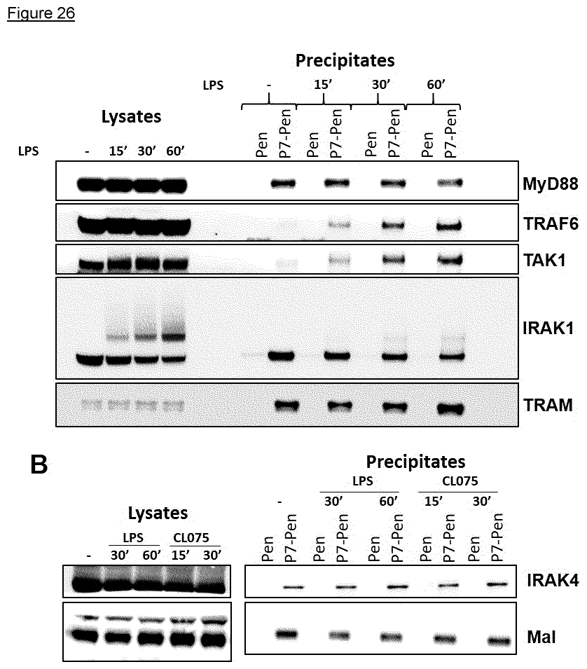

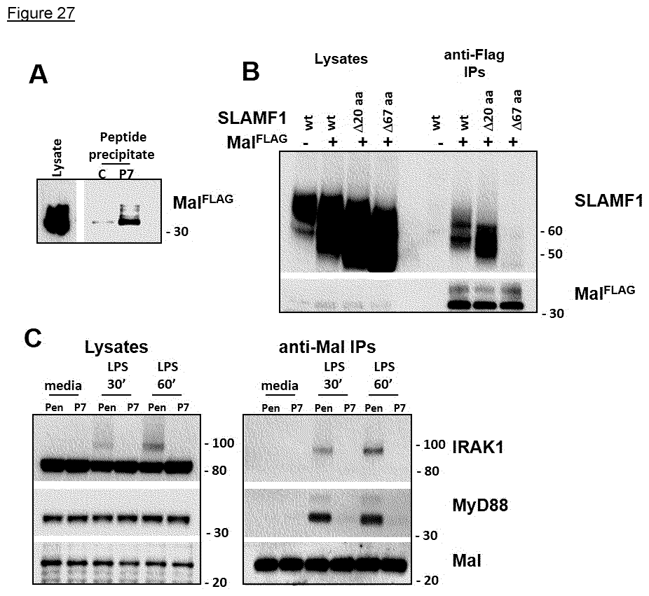

Like TRAM, Mal comprises a TIR domain. As detailed above, the SLAM F1 C-terminus (and also thus peptide P7) interacts with a region of TRAM at the start of its TIR domain. Mal also comprises a TIR domain, the sequence at the start of which was compared to the corresponding SLAMF1-binding sequence of TRAM. The two protein regions displayed low sequence homology (not shown), but are predicted by PHYRE2 modelling to display structural homology (not shown). MyD88 does not co-precipitate with SLAM F1 (Yurchenko et al., supra), but Mal/TIRAP was not previously tested. Thus, we examined if P7-Pen could precipitate overexpressed Mal protein from HEK293T cells, and this was found to be the case (FIG. 27A). However, we found that Mal co-precipitation is mediated by a different sequence in SLAMF1ct than is TRAM co-precipitation with SLAMF1 (FIG. 27B). A SLAMF1 deletion mutant lacking the 20 C-terminal amino acids was able to co-precipitate Mal (FIG. 27B).

At the same time, pre-incubation of macrophages with P7-Pen resulted in the disruption of Mal recruitment to the MyD88 complex and IRAK1 (FIG. 27C).

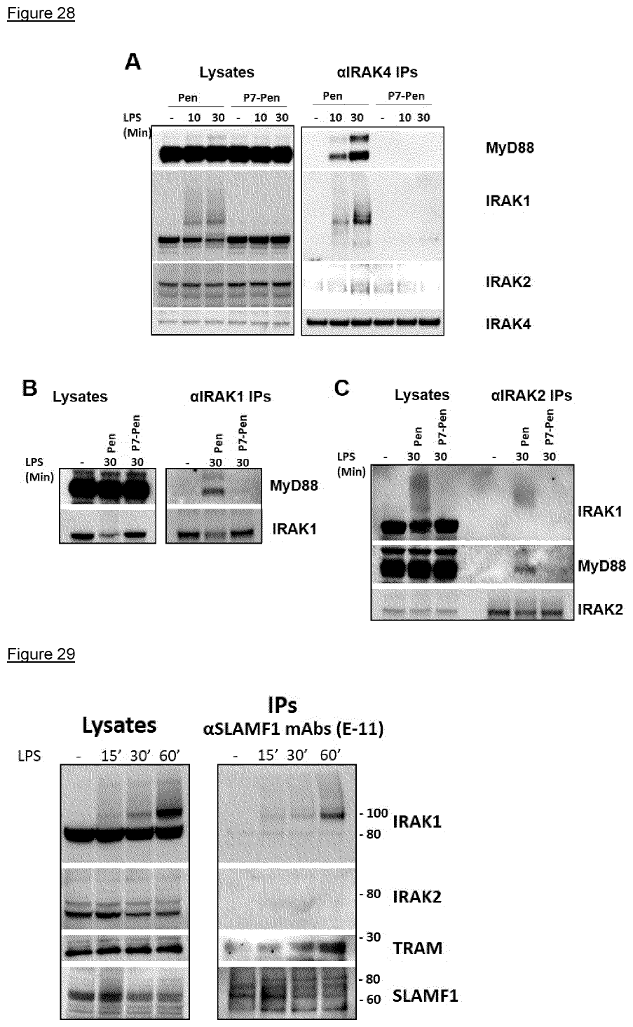

Pre-treatment of monocytes with P7-Pen abrogated recruitment of all tested IRAKs (1,2 and 4) to MyD88 (FIG. 28). This could be achieved by the peptide disrupting the Mal-MyD88 interaction, Mal-IRAK interactions or MyD88-IRAK interactions. Since IL-1R-initiated signalling through the Myddosome complex is not inhibited by the P7 peptide (see above), it is most likely that P7 interferes with Mal-IRAK interactions or the Mal-MyD88 interaction, rather than MyD88-IRAK interactions (since Mal is not involved in IL-1R signaling).

Indeed, a potential P7-Pen interaction motif is located within the protein kinase domain of IRAK4. Alignment of IRAKs with the TRAM-derived sequence that is targeted by the peptide identified sequences in IRAK1 and IRAK4 that are highly similar to the P7-interacting motif in TRAM/TICAM2 (not shown). In endogenous IPs, SLAMF1 was found to interact with modified IRAK1, but not with IRAK2, upon LPS stimulation (FIG. 29). IRAK4 also co-precipitated with GST-SLAMF1ct in GST pull down assays from lysates of primary human macrophages (data not shown).

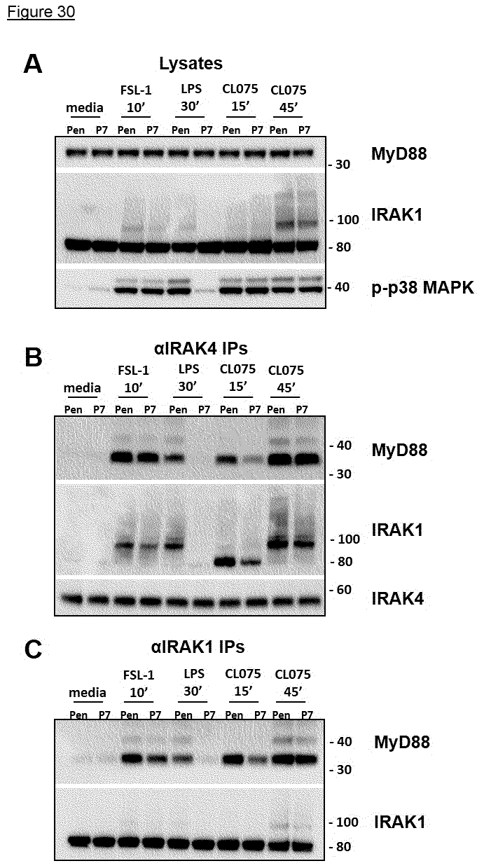

When we tested the effect of the P7 peptide on endogenous IPs of IRAKs after ligation of TLR2 or TLR8, we found that the P7 peptide does not inhibit phosphorylation of p38 MAPK, posttranslational modifications of IRAK1 in TLR2-stimulated cells, or TLR2-mediated recruitment of IRAK1 and 4 to MyD88 (FIG. 30). In TLR8-stimulated cells, IRAK1 modifications did not take place at the early time point (15′), but downstream activation of p38MAPK and IRAK1 and IRAK4 recruitment to MyD88 still took place (FIG. 30). P7-Pen had an inhibitory effect on IRAK1 and IRAK4 recruitment to MyD88 at an early time point in TLR8-stimulated cells, but had no effect on late recruitment of IRAKs or phosphorylation of p38 MAPKs at any analysed time points (FIG. 30). This effect of P7-Pen on TLR8-mediated signaling could indicate that early events are important for efficient initiation of IFNβ expression by TLR8, since the P7 peptide inhibited TLR8-mediated IFNβ expression observed in human PB monocytes and in a whole blood system (see above).

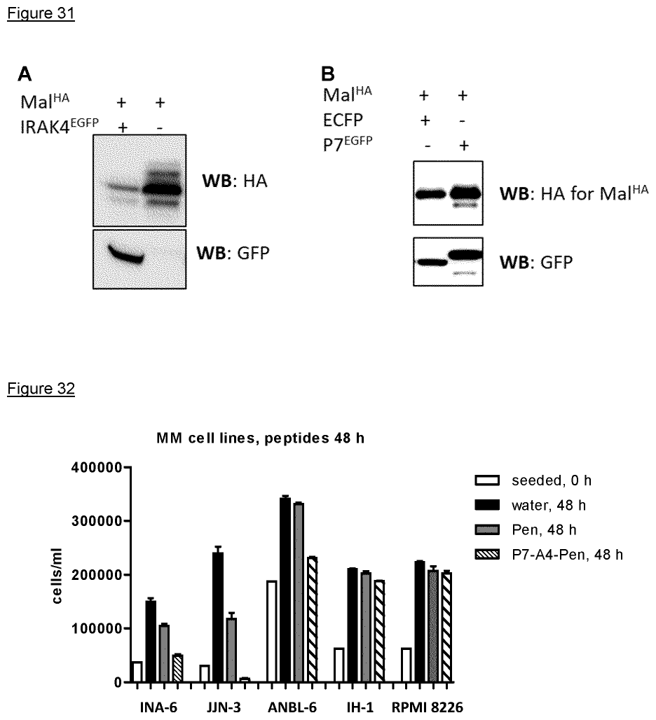

IRAKs 1 and 4 could be responsible for phosphorylation of Mal after LPS signalling. In an overexpression system this interaction can target Mal for degradation (Dunne et al., Journal of Biological Chemistry 285(24): 18276-18282 (2010)). Although this has not been demonstrated with endogenous proteins, phosphorylation of Mal by IRAK1 and/or 4 could be an important step in Myddosome formation. Inhibition of IRAK 1 and 4 kinase activity results in the stabilisation of Mal protein in HEK293 cells (Dunne et al., supra), and so does ECFP-Pep7 when co-expressed with Mal in HEK293 cells (FIG. 31), taking into consideration that HEK cells express endogenous IRAK1 and 4.

It is possible that P7 interacts with the Mal-IRAK1 and/or IRAK4 complex, and blocks the protein kinase activities of IRAKs 1 and/or 4. This would result in the inhibition of all signalling pathways initiated via TLR4, and some signalling from other TLRs.

### Example 13—Effect of P7 on Cancer Cells

To evaluate the potential impact of the P7 peptide on proliferation of tumour cells of lymphoid origin, we performed a CellTiter-Glo Luminescent Cell Viability Assay to address proliferation of several multiple myeloma (MM) cell lines. For the assay, cells were incubated for 48 h in respective media containing either solvent (water), 10 μM control peptide (Pen) or the SLAMF1-derived peptide P7-A4-Pen for 48 h (FIG. 32) (with the exception of the INA-6 cells which were incubated with 12.5 μM control peptide or P7-A4-Pen). In tested cells (JJN-3, ANBL-6, INA-6, IH-1 and RPMI 8226) the P7-A4-Pen peptide inhibited cell proliferation, and in the context of the JJN-3, ANBL-6 and INA-6 lines the P7-A4-Pen peptide strongly inhibited cell proliferation (FIG. 32). The P7-A4-Pen peptide induced cell death of JJN-3 cells (LDH assay, not shown and FIG. 32). This effect could be mediated by P7-A4-Pen-induced inhibition of signalling pathways that are crucial for driving proliferation and/or survival of MM tumour cells. The difference in results obtained with different multiple myeloma cell lines suggests the peptides act through a particular target which is expressed by some of the cell lines but not others.

### Example 14—Testing of Further Peptides

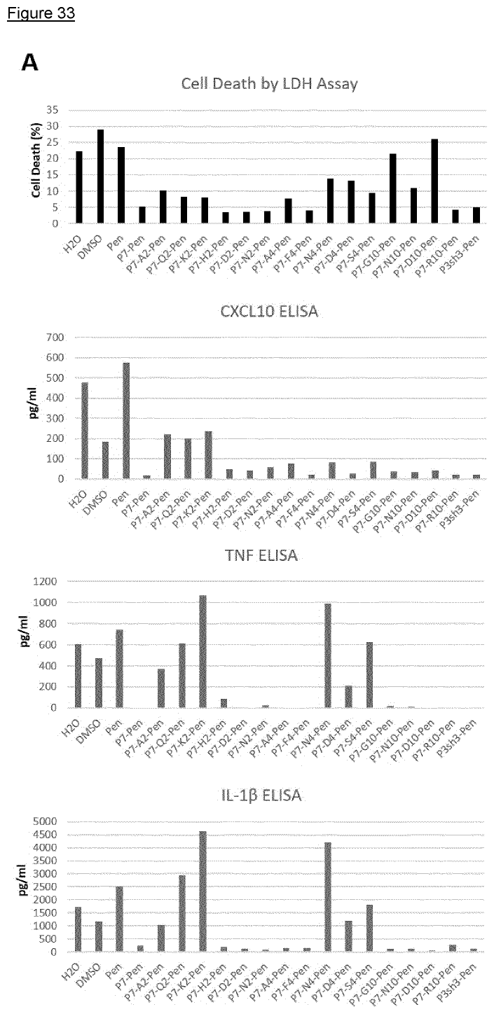

A number of additional peptides were synthesised, as set out in Table 5, and their effect on THP-1 cell responses to LPS stimulation tested as previously (FIG. 33).

TABLE 5

| | | SEQ |
|---|---|---|
| | | ID |
| Name | Sequence | NO |
| P3sh3-Pen | SITVYASVTLTGRQIKIWFQNRRMKWKK | 105 |
| P7-Q2-Pen | IQVYASVTLTGRQIKIWFQNRRMKWKK | 106 |
| P7-K2-Pen | IKVYASVTLTGRQIKIWFQNRRMKWKK | 107 |
| P7-H2-Pen | IHVYASVTLTGRQIKIWFQNRRMKWKK | 108 |
| P7-D2-Pen | IDVYASVTLTGRQIKIWFQNRRMKWKK | 109 |
| P7-F4-Pen | ITVFASVTLTGRQIKIWFQNRRMKWKK | 110 |
| P7-N4-Pen | ITVNASVTLTGRQIKIWFQNRRMKWKK | 111 |
| P7-D4-Pen | ITVDASVTLTGRQIKIWFQNRRMKWKK | 112 |
| P7-S4-Pen | ITVSASVTLTGRQIKIWFQNRRMKWKK | 113 |
| P7-G10- | ITVYASVTLGGRQIKIWFQNRRMKWKK | 114 |
| Pen | | |
| P7-N10- | ITVYASVTLNGRQIKIWFQNRRMKWKK | 115 |
| Pen | | |
| P7-D10- | ITVYASVTLDGRQIKIWFQNRRMKWKK | 116 |
| Pen | | |
| P7-R10- | ITVYASVTLRGRQIKIWFQNRRMKWKK | 117 |
| Pen | | |

As shown in FIG. 33, as expected P3sh3-Pen displayed similar activity to the lead candidate P7-Pen. Additionally, the T2D, T2H, Y4F, T10R and T10N substitutions were found to be well tolerated, with the P7-D2-Pen, P7-H2-Pen, P7-F4-Pen, P7-R10-Pen and P7-N10-Pen displaying similar or even improved activity relative to P7-Pen. P7-D10-Pen and P7-G10-Pen displayed high levels of activity with respect to knocking down cytokine expression, but were found to display relatively high levels of cytotoxicity.

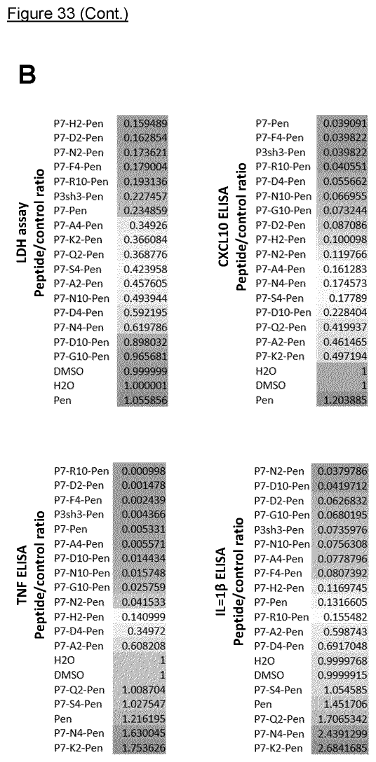

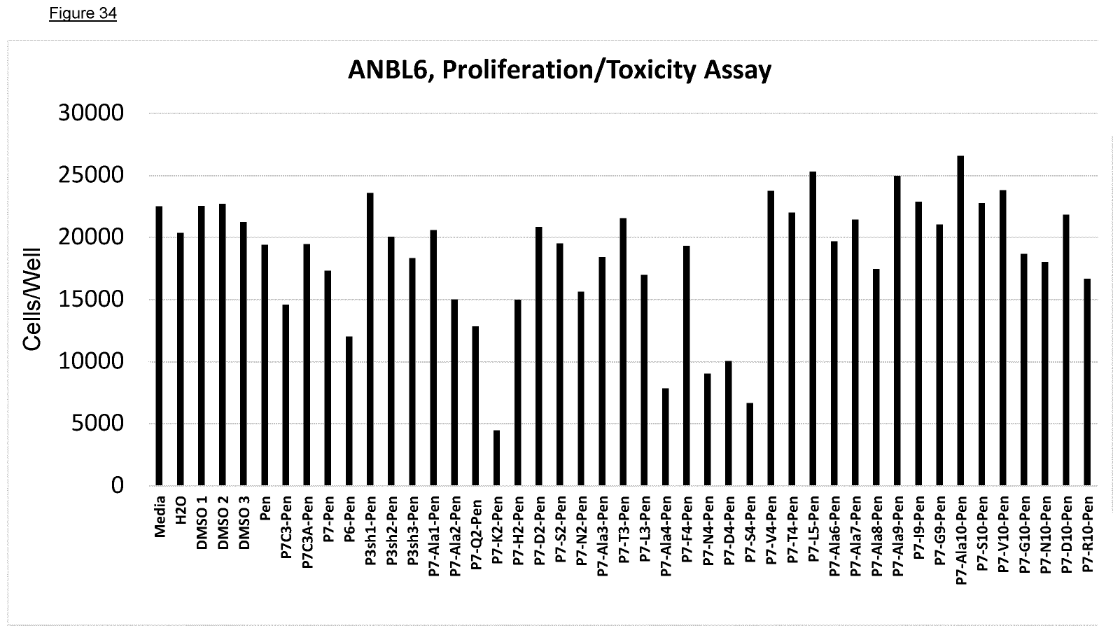

The further peptides, and others described previously, were also tested for their effect on ANBL-6 multiple myeloma cells, in the same manner as described above in Example 13, except the peptides were applied at the higher concentration of 15 μM. The results are shown in FIG. 34. Surprisingly, the peptides found to have an anti-proliferative effect on the ANBL-6 cells (P7-K2-Pen, P7-A4-Pen, P7-N4-Pen, P7-D4-Pen and P7-S4-Pen) were not the same as those found to inhibit TLR signalling (with the exception of P7-A4-Pen, which has both effects). This suggests that the anti-proliferative effect of these peptides is not via a TLR signalling blockade as demonstrated above, but is rather via a different, as-yet-undetermined mechanism.

### Example 15—Concentration Effect on Anti-Proliferative Activity

The peptides P7-Pen, C3A-Pen, P6-Pen, P-P6-Pen (which corresponds to P6-Pen in which the tyrosine at position 4 of P6 is modified to phosphorylated tyrosine) P7-A4-Pen, P7-N4-Pen, P7-D4-Pen and P7-K2-Pen were applied to ANBL-6 cells as described above in Example 13, at a series of concentrations (5, 10, 12.5, 15, 20 and 30 μM; or 5, 10 and 20 μM) to determine the impact of peptide concentration on their anti-proliferative activity. As shown in FIG. 35, both P7-Pen and the control peptide C3A-Pen had essentially no anti-proliferative effect at any concentration; P6 and P-P6-Pen displayed a moderate anti-proliferative at only the highest tested concentration (20 μM); P7-A4-Pen, P7-N4-Pen, P7-D4-Pen and P7-K2-Pen not only showed a clear concentration-dependent effect, but also displayed cytotoxic activity against the cells, with the total cell numbers after 48 hours falling below the number originally seeded at higher concentrations of these peptides.

### Example 16—Combination of Anti-Proliferative Peptides and Melphalan

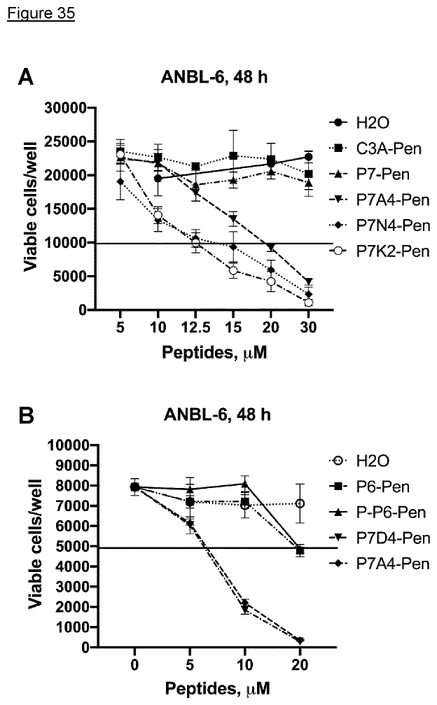

The effect of the anti-proliferative peptide P7-A4-Pen on multiple myeloma ANBL-6 cells, and the effect of the anti-proliferative peptide P7-N4-Pen on multiple myeloma JJN-3 cells, were tested in combination with the licensed multiple myeloma drug melphalan, and the results compared to those obtained with melphalan alone or the peptide alone. As shown in FIG. 36, combination of either peptide with melphalan provided a much-enhanced cytotoxic effect relative to either peptide or melphalan alone. At low melphalan concentrations a synergistic effect was seen with a greater reduction in cell numbers seen with melphalan in combination with P7-A4-Pen or P7-N4-Pen than would have been obtained from the cumulative effects of the two compounds. If these in vitro results correlate to the clinical effect of combining melphalan with an anti-proliferative oligopeptidic compound of the invention, this could allow the administration of melphalan at a much lower dosage than is currently used for melphalan monotherapy (e.g. at least 50% lower than is currently used), particularly for multiple myeloma treatment. Such a reduction in required melphalan dosage would be highly advantageous as melphalan is highly toxic.

### Example 17—Effect of Anti-Proliferative Peptides on Other Cancer Cell Lines

To investigate whether the anti-proliferative peptides of the invention have a broader anti-proliferative effect against other types of cancer cells (beyond multiple myeloma), P7-Pen, P7-A4-Pen and P7-N4-Pen were tested against the acute myeloid leukaemia cell line THP-1 (and compared to the control peptide C3A-Pen), and P7-Pen and P7-N4-Pen were tested against the T cell leukaemia Jurkat cell line and the SW480 colon cancer cell line with the Penetratin peptide as control, using the methods as set out above. The peptides were tested against the SW480 cell line in the presence and absence of LPS.

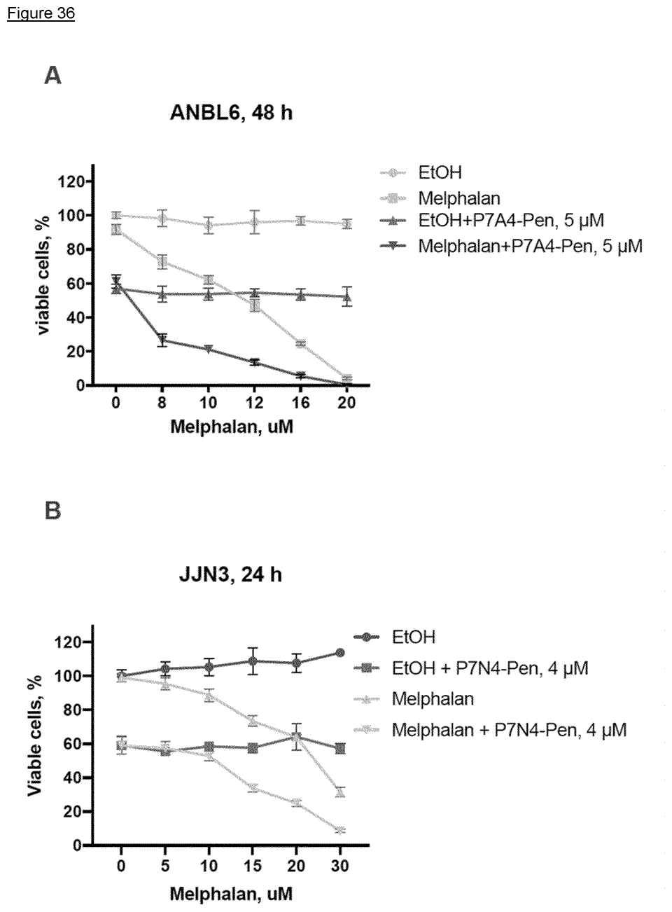

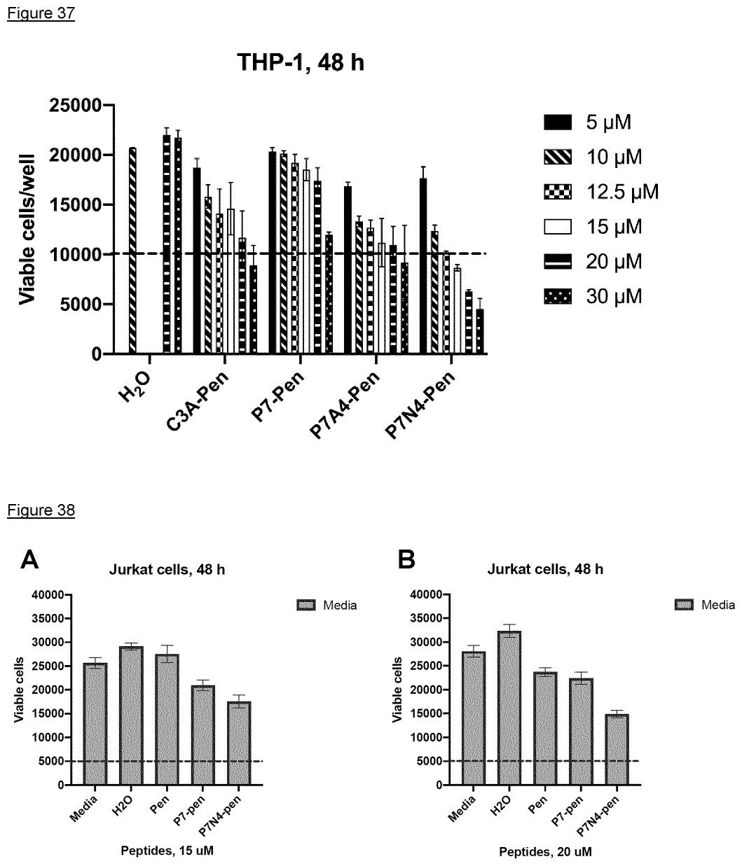

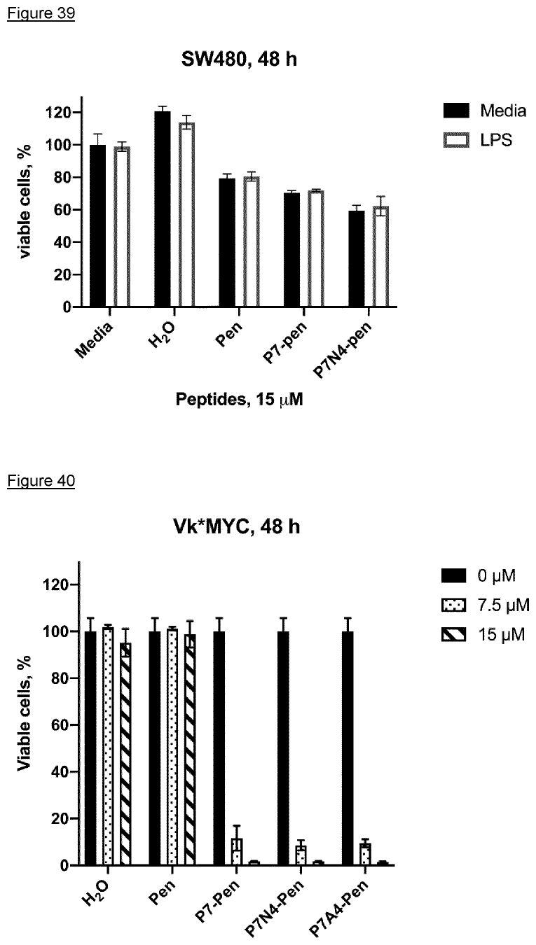

As shown in FIG. 37, the P7-A4-Pen, and particularly the P7-N4-Pen peptides had an anti-proliferative effect on THP-1 cells, with the number of cells reduced below the number seeded at higher peptide concentrations. Both P7-Pen and P7-N4-Pen also displayed a clear anti-proliferative effect on Jurkat cells (FIG. 38). The results suggest a broad applicability of the anti-proliferative peptides of the invention against haematological cancers. P7-Pen and P7-N4-Pen also displayed a degree of anti-proliferative effect against the SW480 cell line (FIG. 39), indicating that the anti-proliferative peptides of the invention are also useful against solid cancers. The presence of LPS had no impact on the effect of the peptides on SW480 cells.

While the anti-proliferative peptides had a lesser effect on some cell lines (e.g. SW480) than others, the results nonetheless indicate a potential utility in treatment of these cancers, particularly when used within a combination therapy.

### Example 18—Effect of Peptides on Primary Multiple Myeloma Cells

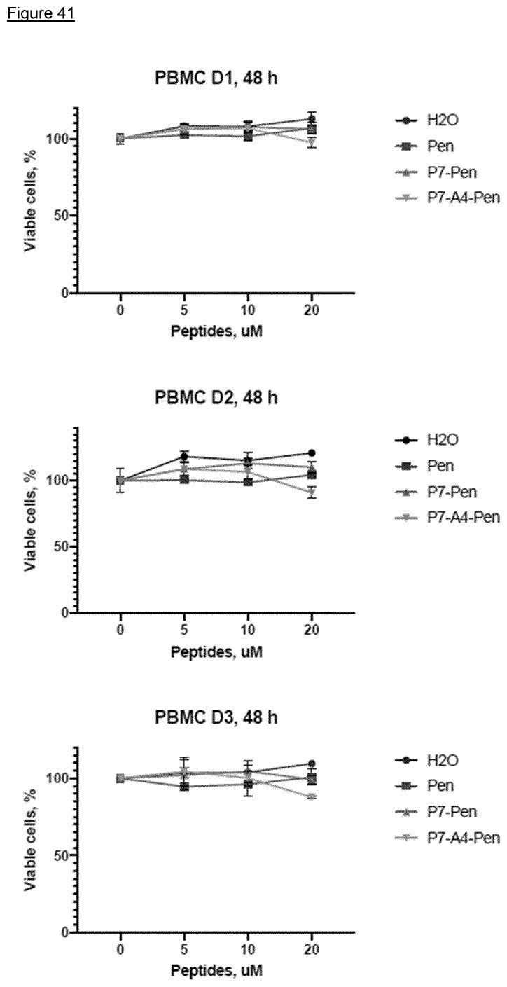

Primary multiple myeloma cells were isolated from Vk*Myc mice and provided by the Multiple Myeloma Research Group, IKOM, NTNU. The peptides P7-Pen, P7-N4-Pen and P7-A4-Pen were applied to the cultured primary cells and effect of the peptides on cell growth analysed as previously. All three peptides displayed a strong cytotoxic effect on primary multiple myeloma cells (FIG. 40). The Vk*Myc mouse model has previously been shown to be highly predictive for clinical efficacy of multiple myeloma treatments (Chesi et al., Blood 120(2): 376-385, 2012), demonstrating the high potential of the anti-proliferative peptides of the invention in multiple myeloma therapy.

### Example 19—Toxicity of Anti-Proliferative Peptides

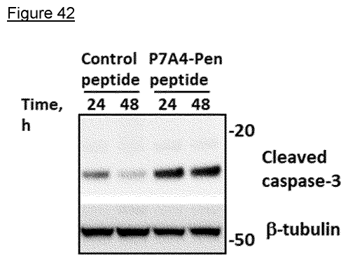

The toxicities of the anti-proliferative peptides P7-Pen and P7-A4-Pen were tested against PBMCs from three healthy donors (PBMCs isolated as described above). P7-Pen and P7-A4-Pen were applied to the PBMCs for 48 hours at a range of concentrations up to 20 μM. No toxicity was seen against the PBMCs from any of the three donors (FIG. 41), demonstrating that the anti-proliferative peptides are not generally cytotoxic, but rather display selective cytotoxicity for cancer cells.

### Example 20—Caspase Activation by Anti-Proliferative Peptides

To investigate the mechanism of cytotoxicity mediated by the anti-proliferative peptides of the invention, the effect of the peptide P7-A4-Pen on caspase-3 activation in JJN-3 multiple myeloma cells was investigated. Caspase-3 is a pro-apoptotic caspase which is activated by cleavage by upstream caspases 8, 9 and 10 in response to cell death stimuli. Activated, cleaved caspase-3 cleaves and activates caspases 6 and 7, which induce apoptosis. JJN-3 multiple myeloma cells were incubated with 10 μM P7-A4-Pen or Penetratin control for 24 or 48 hours, lysed and analysed by Western blot to investigate caspase-3 activation (by blotting cleaved caspase-3).

As shown in FIG. 42, P7-A4-Pen strongly stimulates caspase-3 cleavage, demonstrating that the peptide induces apoptosis in multiple myeloma cells, by a currently unknown mechanism.

1. An oligopeptidic compound comprising:
a) a first oligopeptidic component which:
i) is capable of blocking signalling from TLR4, TLR7, TLR8 and/or TLR9; and/or
ii) has an anti-proliferative and/or cytotoxic effect on cancer cells;
and comprising the amino acid sequence set forth in SEQ ID NO: 1, or an amino acid sequence having at least 70% sequence identity thereto; and
b) a second oligopeptidic component, which is a cytosol-targeting cell penetrating oligopeptidic component.

2. The oligopeptidic compound of claim 1, wherein the first oligopeptidic component has an anti-proliferative and/or cytotoxic effect on ANBL6 multiple myeloma cells and/or VK-MYC multiple myeloma cells.

3. The oligopeptidic compound of claim 1 or 2, where the first oligopeptidic component is 8-13 amino acids long, preferably 9-11 amino acids long.

4. The oligopeptidic compound of any one of claims 1 to 3, wherein the first oligopeptidic component
(i) is not extended at its C-terminus relative to SEQ ID NO: 1 and/or
(ii) comprises an N-terminal extension of up to 3 amino acids relative to SEQ ID NO: 1.

5. The oligopeptidic compound of any one of claim 1 to 3 or 4(i), wherein the first oligopeptidic component is not extended at its N-terminus relative to SEQ ID NO: 1.

6. The oligopeptidic compound of any one of claims 1 to 5, wherein the second oligopeptidic component is located C-terminal to the first oligopeptidic component.

7. The oligopeptidic compound of any one of claims 1 to 6, wherein the first oligopeptidic component comprises 1 to 3 amino acid substitutions relative to SEQ ID NO: 1, and said substitutions are located at positions 2, 4 and/or 10 of SEQ ID NO: 1.

8. The oligopeptidic compound of claim 7, wherein the threonine at position 2 of SEQ ID NO: 1 is substituted for asparagine, aspartic acid, histidine or lysine; the tyrosine at position 4 of SEQ ID NO: 1 is substituted for alanine, phenylalanine, asparagine, aspartic acid or serine; and/or the threonine at position 10 of SEQ ID NO: 1 is substituted for proline, asparagine or arginine.

9. The oligopeptidic compound of any one of claims 1 to 6, wherein the first oligopeptidic component consists of the amino acid sequence set forth in SEQ ID NO: 1.

10. The oligopeptidic compound of any one of claims 1 to 8, wherein the first oligopeptidic component consists of the amino acid sequence set forth in any one of SEQ ID NOs: 2-6, 16-20, 104 or 118-121.

11. The oligopeptidic compound of any one of claims 1 to 10, wherein the second oligopeptidic component:
(i) is a polyarginine peptide, wherein preferably the polyarginine peptide consists of the amino acid sequence set forth in SEQ ID NO: 7;
(ii) is penetratin, consisting of the amino acid sequence set forth in SEQ ID NO: 8, or an amino acid sequence having at least 80% sequence identity thereto; or
(iii) consists of the amino acid sequence set forth in SEQ ID NO: 9, or an amino acid sequence having at least 80% sequence identity thereto.

12. The oligopeptidic compound of any one of claims 1 to 11, wherein the first oligopeptidic component and the second oligopeptidic component are joined by a linker, optionally wherein the linker is a glycine residue.

13. The oligopeptidic compound of any one of claims 1 to 12, comprising an amino acid sequence as set forth in any one of SEQ ID NOs: 10-15, 107-113, 115 or 117.

14. A pharmaceutical composition comprising an oligopeptidic compound as defined in any one of claims 1 to 13, and one or more pharmaceutically-acceptable diluents, carriers or excipients.

15. An oligopeptidic compound as defined in any one of claims 1 to 13, or a composition as defined in claim 14, for use in therapy.

16. An oligopeptidic compound as defined in any one of claims 1 to 13, wherein the first oligopeptidic component of the oligopeptidic compound is capable of blocking signalling from TLR4, TLR7, TLR8 and/or TLR9, or a composition as defined in claim 14 comprising said oligopeptidic compound, for use in treatment or prevention of a disease associated with hypercytokinemia in a human subject, wherein preferably said hypercytokinemia is characterised by up-regulation of expression of interferon 13, IL-1β, IL-6 and/or TNF.

17. The oligopeptidic compound or composition for use according to claim 16, wherein the first oligopeptidic component consists of the amino acid sequence set forth in any one of SEQ ID NOs: 1-6, 16-20 or 104.

18. The oligopeptidic compound or composition for use according to claim 17, comprising an amino acid sequence as set forth in any one of SEQ ID NOs: 10-15, 108-110, 115 or 117.

19. The oligopeptidic compound or composition for use according to any one of claims 16 to 18, wherein said disease is sepsis.

20. The oligopeptidic compound or composition for use according to any one of claims 16 to 19, wherein said disease is associated with infection by a bacterium and/or virus.

21. The oligopeptidic compound or composition for use according to any one of claims 16 to 20, wherein said treatment or prevention further comprises administration to the subject of a second therapeutically active agent, preferably wherein the second therapeutically active agent is an antibiotic or an antiviral agent.

22. An oligopeptidic compound as defined in any one of claims 1 to 13, wherein the first oligopeptidic component of the oligopeptidic compound is capable of blocking signalling from TLR4, TLR7, TLR8 and/or TLR9, or a composition as defined in claim 14 comprising said oligopeptidic compound, for use in treatment or prevention of an inflammatory disease, an autoimmune disease or ischemia-reperfusion injury;
preferably wherein the oligopeptidic compound or composition is as defined in claim 17 or 18.

23. The oligopeptidic compound or composition for use according to claim 22, wherein the inflammatory disease or autoimmune disease is selected from type II diabetes, a neurodegenerative disease, preferably Alzheimer's disease or Parkinson's disease, gout, non-alcoholic steatohepatitis, inflammatory bowel disease, rheumatoid arthritis, systemic lupus erythematosus, Schnitzler's syndrome, atherosclerosis, graft-versus-host disease, cryopyrin-associated periodic syndromes or a fibrotic disease.

24. An oligopeptidic compound as defined in any one of claims 1 to 13, wherein the first oligopeptidic component of the oligopeptidic compound has an anti-proliferative and/or cytotoxic effect on cancer cells, or a composition as defined in claim 14 comprising said oligopeptidic compound, for use in the treatment of cancer.

25. The oligopeptidic compound or composition for use according to claim 24, wherein the first oligopeptidic component consists of the amino acid sequence set forth in any one of SEQ ID NOs: 1-3 or 118-121.

26. The oligopeptidic compound or composition for use according to claim 25, comprising an amino acid sequence as set forth in any one of SEQ ID NOs: 10-14, 107 and 111-113.

27. The oligopeptidic compound or composition for use according to any one of claims 24 to 26, wherein the cancer is breast cancer, hepatocellular carcinoma, head and neck squamous cell carcinoma, lung cancer, melanoma, endometrial cancer, a haematological cancer, cervical cancer, ovarian cancer, colorectal cancer or pancreatic cancer.

28. The oligopeptidic compound or composition for use according to claim 27, wherein the haematological cancer is multiple myeloma, acute myeloid leukaemia, T cell leukaemia, Hodgkin lymphoma or non-Hodgkin lymphoma.

29. The oligopeptidic compound or composition for use according to any one of claims 24 to 28, wherein said treatment further comprises administration to the subject of a second therapeutically active agent, preferably wherein the second therapeutically active agent is a chemotherapy, radiotherapy, hormonal therapy and/or immunotherapy agent, and/or a small molecule inhibitor or biologic.

30. The oligopeptidic compound or composition for use according to claim 29, wherein said second therapeutically active agent is melphalan, preferably wherein said treatment is for multiple myeloma.

31. A method of treating or preventing a disease in a human subject, comprising administering to the subject an oligopeptidic compound as defined in any one of claims 1 to 13 or a composition as defined in claim 14; wherein the disease and oligopeptidic compound are as defined in any one of claims 16 to 20 or 22 to 28, and optionally wherein the treatment or prevention is as defined in claim 21, 29 or 30.

32. Use of an oligopeptidic compound as defined in any one of claims 1 to 13 in the manufacture of a medicament for use in the treatment or prevention of a disease in a human subject, wherein the disease and oligopeptidic compound are as defined in any one of claims 16 to 20 or 22 to 28, and optionally wherein the treatment or prevention is as defined in claim 21, 29 or 30.

33. A kit comprising an oligopeptidic compound as defined in any one of claims 1 to 13 or a pharmaceutical composition as defined in claim 14, and a second therapeutically active agent;
preferably wherein the oligopeptidic compound is as defined in any one of claims 16 to 18, and the second therapeutically active agent is an antibiotic or an antiviral agent; or
the oligopeptidic compound is as defined in any one of claims 24 to 26, and the second therapeutically active agent is a chemotherapy, radiotherapy, hormonal therapy and/or immunotherapy agent, and/or a small molecule inhibitor or biologic.

34. A product comprising an oligopeptidic compound as defined in any one of claims 1 to 13 or a pharmaceutical composition as defined in claim 14, and a second therapeutically active agent, as a combined preparation for separate, simultaneous or sequential use in the treatment or prevention of a disease in a human subject, wherein:
(i) the oligopeptidic compound is as defined in any one of claims 16 to 18 and the disease is as defined in any one of claim 16, 19, 20, 22 or 23, and the second therapeutically active agent is preferably an antibiotic or an antiviral agent; or
(ii) the oligopeptidic compound is as defined in any one of claims 24 to 26 and the disease is as defined in any one of claim 24, 27 or 28, and the second therapeutically active agent is preferably a chemotherapy, radiotherapy, hormonal therapy and/or immunotherapy agent, and/or a small molecule inhibitor or biologic.

35. A nucleic acid molecule comprising a nucleotide sequence which encodes an oligopeptidic compound as defined in any one of claims 1 to 13.

36. A construct comprising the nucleic acid molecule of claim 35, or a vector comprising said construct or nucleic acid molecule.

37. A method of downregulating expression of interferon β by a human cell, comprising contacting the cell with an oligopeptidic compound as defined in any one of claims 1 to 13, wherein the first oligopeptidic component of the oligopeptidic compound is capable of blocking signalling from TLR4, TLR7, TLR8 and/or TLR9; wherein optionally expression of IL-1β, TNF, IL-6 and/or CXCL10 is also downregulated.


SEQUENCE LISTING (121 sequences)

SEQ ID NO: 1 - protein, 10 aa, Artificial Sequence
  P7
ITVYASVTLT

SEQ ID NO: 2 - protein, 10 aa, Homo sapiens
ITVYASVTLP

SEQ ID NO: 3 - protein, 10 aa, Artificial Sequence
  P7-A4
ITVAASVTLT

SEQ ID NO: 4 - protein, 10 aa, Artificial Sequence
  P7-N2
INVYASVTLT

SEQ ID NO: 5 - protein, 13 aa, Artificial Sequence
  P3sh1
TNSITVYASVTLT

SEQ ID NO: 6 - protein, 12 aa, Artificial Sequence
  P3sh2
NSITVYASVTLT

SEQ ID NO: 7 - protein, 9 aa, Artificial Sequence
  Nona-arginine
RRRRRRRRR

SEQ ID NO: 8 - protein, 16 aa, Drosophila melanogaster
RQIKIWFQNRRMKWKK

SEQ ID NO: 9 - protein, 18 aa, Artificial Sequence
  Model Amphipathic Peptide (KLA)
KLALKLALKALKAALKLA

SEQ ID NO: 10 - protein, 27 aa, Artificial Sequence
  P7-Pen
ITVYASVTLTGRQIKIWFQNRRMKWKK

SEQ ID NO: 11 - protein, 27 aa, Artificial Sequence
  P6-Pen
ITVYASVTLPGRQIKIWFQNRRMKWKK

SEQ ID NO: 12 - protein, 29 aa, Artificial Sequence
  P7-KLA
ITVYASVTLTGKLALKLALKALKAALKLA

SEQ ID NO: 13 - protein, 20 aa, Artificial Sequence
  P7-Arg9
ITVYASVTLTGRRRRRRRRR

SEQ ID NO: 14 - protein, 27 aa, Artificial Sequence
  P7-A4-Pen
ITVAASVTLTGRQIKIWFQNRRMKWKK

SEQ ID NO: 15 - protein, 27 aa, Artificial Sequence
  P7-N2-Pen
INVYASVTLTGRQIKIWFQNRRMKWKK

SEQ ID NO: 16 - protein, 11 aa, Artificial Sequence
  P3sh3
SITVYASVTLT

SEQ ID NO: 17 - protein, 10 aa, Artificial Sequence
  P7-D2
IDVYASVTLT

SEQ ID NO: 18 - protein, 10 aa, Artificial Sequence
  P7-H2
IHVYASVTLT

SEQ ID NO: 19 - protein, 10 aa, Artificial Sequence
  P7-F4
ITVFASVTLT

SEQ ID NO: 20 - protein, 10 aa, Artificial Sequence
  P7-R10
ITVYASVTLR

SEQ ID NO: 21 - protein, 7 aa, Drosophila melanogaster
RRMKWKK

SEQ ID NO: 22 - protein, 8 aa, Drosophila melanogaster
NRRMKWKK

SEQ ID NO: 23 - protein, 9 aa, Drosophila melanogaster
QNRRMKWKK

SEQ ID NO: 24 - protein, 10 aa, Drosophila melanogaster
FQNRRMKWKK

SEQ ID NO: 25 - protein, 7 aa, Artificial Sequence
  Penetratin Derivative
RREKWKK

SEQ ID NO: 26 - protein, 7 aa, Artificial Sequence
  Penetratin Derivative
RRQKWKK

SEQ ID NO: 27 - protein, 7 aa, Artificial Sequence
  Penetratin Derivative
KRMKWKK

SEQ ID NO: 28 - protein, 7 aa, Artificial Sequence
  Penetratin Derivative
RKMKWKK

SEQ ID NO: 29 - protein, 7 aa, Artificial Sequence
  Penetratin Derivative
  MOD_RES (3)..(3): Orn
RRXKWKK

SEQ ID NO: 30 - protein, 7 aa, Artificial Sequence
  Penetratin Derivative
RRMKQKK

SEQ ID NO: 31 - protein, 7 aa, Artificial Sequence
  Penetratin Derivative
RRMKWFK

SEQ ID NO: 32 - protein, 7 aa, Artificial Sequence
  Penetratin Derivative
  MOD_RES (2)..(2): Orn
RXRKWKK

SEQ ID NO: 33 - protein, 7 aa, Artificial Sequence
  Penetratin Derivative
RRMWKKK

SEQ ID NO: 34 - protein, 7 aa, Artificial Sequence
  Penetratin Derivative
RRMKKWK

SEQ ID NO: 35 - protein, 18 aa, Artificial Sequence
  Pegelin (Protegrin Derivative)
RGGRLSYSRRRFSTSTGR

SEQ ID NO: 36 - protein, 13 aa, Human immunodeficiency virus type 1
GRKKRRQRRRPPQ

SEQ ID NO: 37 - protein, 11 aa, Human immunodeficiency virus type 1
YGRKKRRQRRR

SEQ ID NO: 38 - protein, 33 aa, human herpesvirus 1
DAATATRGRSAASRPTERPRAPARSASRPRRVD

SEQ ID NO: 39 - protein, 27 aa, Artificial Sequence
  Transportan
GWTLNSAGYLLGKINLKALAALAKKIL

SEQ ID NO: 40 - protein, 21 aa, Artificial Sequence
  Transportan-10
AGYLLGKINLKALAALAKKIL

SEQ ID NO: 41 - protein, 30 aa, Artificial Sequence
  KALA Amphipathic Peptide
WEAKLAKALAKALAKHLAKALAKALKACEA

SEQ ID NO: 42 - protein, 27 aa, Artificial Sequence
  MPG CPP
GALFLGFLGAAGSTMGAWSQPKSKRKV

SEQ ID NO: 43 - protein, 19 aa, Artificial Sequence
  Vectocell Peptide DPV1047
VKRGLKLRHVRPRVTRMDV

SEQ ID NO: 44 - protein, 14 aa, Homo sapiens
SRRARRSPRHLGSG

SEQ ID NO: 45 - protein, 16 aa, Artificial Sequence
  Vectocell Peptide DPV15
LRRERQSRLRRERQSR

SEQ ID NO: 46 - protein, 22 aa, Artificial Sequence
  Vectocell Peptide DPV15b
GAYDLRRRERQSRLRRRERQSR

SEQ ID NO: 47 - protein, 7 aa, Artificial Sequence
  Hepta-arginine
RRRRRRR

SEQ ID NO: 48 - protein, 11 aa, Artificial Sequence
  Undeca-arginine
RRRRRRRRRRR

SEQ ID NO: 49 - protein, 15 aa, Artificial Sequence
  P3
TNSITVYASVTLTES

SEQ ID NO: 50 - protein, 10 aa, Artificial Sequence
  P7C3
IATYASTALT

SEQ ID NO: 51 - protein, 22 aa, Artificial Sequence
  P7-Arg11
ITVYASVTLTGRRRRRRRRRRR

SEQ ID NO: 52 - protein, 22 aa, Artificial Sequence
  Arg11-P7
RRRRRRRRRRRGITVYASVTLT

SEQ ID NO: 53 - protein, 22 aa, Artificial Sequence
  P7C3-Arg11
IATYASTALTGRRRRRRRRRRR

SEQ ID NO: 54 - protein, 20 aa, Artificial Sequence
  P7C3-Arg9
IATYASTALTGRRRRRRRRR

SEQ ID NO: 55 - protein, 18 aa, Artificial Sequence
  P7-Arg7
ITVYASVTLTGRRRRRRR

SEQ ID NO: 56 - protein, 27 aa, Artificial Sequence
  Pen-P7
RQIKIWFQNRRMKWKKGITVYASVTLT

SEQ ID NO: 57 - protein, 27 aa, Artificial Sequence
  P7C3-Pen
IATYASTALTGRQIKIWFQNRRMKWKK

SEQ ID NO: 58 - protein, 29 aa, Artificial Sequence
  P7-Pegelin
ITVYASVTLTGRGGRLSYSRRRFSTSTGR

SEQ ID NO: 59 - protein, 24 aa, Artificial Sequence
  P7-TAT
ITVYASVTLTGGRKKRRQRRRPPQ

SEQ ID NO: 60 - protein, 29 aa, Artificial Sequence
  KLA-P7
KLALKLALKALKAALKLAGITVYASVTLT

SEQ ID NO: 61 - protein, 29 aa, Artificial Sequence
  P7C3-KLA
IATYASTALTGKLALKLALKALKAALKLA

SEQ ID NO: 62 - protein, 30 aa, Artificial Sequence
  P7-DPV1047
ITVYASVTLTGVKRGLKLRHVRPRVTRMDV

SEQ ID NO: 63 - protein, 19 aa, Artificial Sequence
  P7-Pen(Sh)
ITVYASVTLTGNRRMKWKK

SEQ ID NO: 64 - protein, 27 aa, Artificial Sequence
  P3-Arg11
TNSITVYASVTLTESGRRRRRRRRRRR

SEQ ID NO: 65 - protein, 30 aa, Artificial Sequence
  P3sh1-Pen
TNSITVYASVTLTGRQIKIWFQNRRMKWKK

SEQ ID NO: 66 - protein, 29 aa, Artificial Sequence
  P3sh2-Pen
NSITVYASVTLTGRQIKIWFQNRRMKWKK

SEQ ID NO: 67 - protein, 28 aa, Artificial Sequence
  P10-Pen
ITVYASVTLPEGRQIKIWFQNRRMKWKK

SEQ ID NO: 68 - protein, 28 aa, Artificial Sequence
  P10-A11-Pen
ITVYASVTLPAGRQIKIWFQNRRMKWKK

SEQ ID NO: 69 - protein, 28 aa, Artificial Sequence
  P11-Pen
ITVYASVTLTEGRQIKIWFQNRRMKWKK

SEQ ID NO: 70 - protein, 28 aa, Artificial Sequence
  P11-A11-Pen
ITVYASVTLTAGRQIKIWFQNRRMKWKK

SEQ ID NO: 71 - protein, 11 aa, Artificial Sequence
  P10
ITVYASVTLPE

SEQ ID NO: 72 - protein, 11 aa, Artificial Sequence
  P11
ITVYASVTLTE

SEQ ID NO: 73 - protein, 11 aa, Artificial Sequence
  P10-A11
ITVYASVTLPA

SEQ ID NO: 74 - protein, 11 aa, Artificial Sequence
  P11-A11
ITVYASVTLTA

SEQ ID NO: 75 - protein, 27 aa, Artificial Sequence
  P7-A1-Pen
ATVYASVTLTGRQIKIWFQNRRMKWKK

SEQ ID NO: 76 - protein, 27 aa, Artificial Sequence
  P7-A2-Pen
IAVYASVTLTGRQIKIWFQNRRMKWKK

SEQ ID NO: 77 - protein, 27 aa, Artificial Sequence
  P7-S2-Pen
ISVYASVTLTGRQIKIWFQNRRMKWKK

SEQ ID NO: 78 - protein, 27 aa, Artificial Sequence
  P7-A3-Pen
ITAYASVTLTGRQIKIWFQNRRMKWKK

SEQ ID NO: 79 - protein, 27 aa, Artificial Sequence
  P7-T3-Pen
ITTYASVTLTGRQIKIWFQNRRMKWKK

SEQ ID NO: 80 - protein, 27 aa, Artificial Sequence
  P7-L3-Pen
ITLYASVTLTGRQIKIWFQNRRMKWKK

SEQ ID NO: 81 - protein, 27 aa, Artificial Sequence
  P7-V4-Pen
ITVVASVTLTGRQIKIWFQNRRMKWKK

SEQ ID NO: 82 - protein, 27 aa, Artificial Sequence
  P7-T4-Pen
ITVTASVTLTGRQIKIWFQNRRMKWKK

SEQ ID NO: 83 - protein, 27 aa, Artificial Sequence
  P7-L5-Pen
ITVYLSVTLTGRQIKIWFQNRRMKWKK

SEQ ID NO: 84 - protein, 27 aa, Artificial Sequence
  P7-A6-Pen
ITVYAAVTLTGRQIKIWFQNRRMKWKK

SEQ ID NO: 85 - protein, 27 aa, Artificial Sequence
  P7-A7-Pen
ITVYASATLTGRQIKIWFQNRRMKWKK

SEQ ID NO: 86 - protein, 27 aa, Artificial Sequence
  P7-A8-Pen
ITVYASVALTGRQIKIWFQNRRMKWKK

SEQ ID NO: 87 - protein, 27 aa, Artificial Sequence
  P7-A9-Pen
ITVYASVTATGRQIKIWFQNRRMKWKK

SEQ ID NO: 88 - protein, 27 aa, Artificial Sequence
  P7-I9-Pen
ITVYASVTITGRQIKIWFQNRRMKWKK

SEQ ID NO: 89 - protein, 27 aa, Artificial Sequence
  P7-G9-Pen
ITVYASVTGTGRQIKIWFQNRRMKWKK

SEQ ID NO: 90 - protein, 27 aa, Artificial Sequence
  P7-A10-Pen
ITVYASVTAAGRQIKIWFQNRRMKWKK

SEQ ID NO: 91 - protein, 27 aa, Artificial Sequence
  P7-S10-Pen
ITVYASVTLSGRQIKIWFQNRRMKWKK

SEQ ID NO: 92 - protein, 27 aa, Artificial Sequence
  P7-V10-Pen
ITVYASVTLVGRQIKIWFQNRRMKWKK

SEQ ID NO: 93 - protein, 27 aa, Artificial Sequence
  P7C3A-Pen
IATYASVTLTGRQIKIWFQNRRMKWKK

SEQ ID NO: 94 - protein, 10 aa, Artificial Sequence
  FSL-1
  LIPID (1)..(1): Dipalmitoyl
  LIPID (1)..(1): Dipalmitoyl cysteine
CGDPKHPKSF

SEQ ID NO: 95 - protein, 5 aa, Artificial Sequence
  Penta-arginine
RRRRR

SEQ ID NO: 96 - protein, 6 aa, Artificial Sequence
  Hexa-arginine
RRRRRR

SEQ ID NO: 97 - protein, 8 aa, Artificial Sequence
  Octa-arginine
RRRRRRRR

SEQ ID NO: 98 - protein, 10 aa, Artificial Sequence
  Deca-arginine
RRRRRRRRRR

SEQ ID NO: 99 - protein, 10 aa, Artificial Sequence
  ECFP-C1 Plasmid Linker
SGLRSRAQAS

SEQ ID NO: 100 - DNA, 25 bp, Artificial Sequence
  Ligation Sequence
caccgccagctttctggtctcacgc

SEQ ID NO: 101 - DNA, 25 bp, Artificial Sequence
  Ligation Sequence
aaacgcgtgagaccagaaagctggc

SEQ ID NO: 102 - DNA, 24 bp, Artificial Sequence
  Ligation Sequence
caccgtttgtaatcgtcgataccc

SEQ ID NO: 103 - DNA, 24 bp, Artificial Sequence
  Ligation Sequence
aaacgggtatcgacgattacaaac

SEQ ID NO: 104 - protein, 10 aa, Artificial Sequence
  P7-N10
ITVYASVTLN

SEQ ID NO: 105 - protein, 28 aa, Artificial Sequence
  P3sh3-Pen
SITVYASVTLTGRQIKIWFQNRRMKWKK

SEQ ID NO: 106 - protein, 27 aa, Artificial Sequence
  P7-Q2-Pen
IQVYASVTLTGRQIKIWFQNRRMKWKK

SEQ ID NO: 107 - protein, 27 aa, Artificial Sequence
  P7-K2-Pen
IKVYASVTLTGRQIKIWFQNRRMKWKK

SEQ ID NO: 108 - protein, 27 aa, Artificial Sequence
  P7-H2-Pen
IHVYASVTLTGRQIKIWFQNRRMKWKK

SEQ ID NO: 109 - protein, 27 aa, Artificial Sequence
  P7-D2-Pen
IDVYASVTLTGRQIKIWFQNRRMKWKK

SEQ ID NO: 110 - protein, 27 aa, Artificial Sequence
  P7-F4-Pen
ITVFASVTLTGRQIKIWFQNRRMKWKK

SEQ ID NO: 111 - protein, 27 aa, Artificial Sequence
  P7-N4-Pen
ITVNASVTLTGRQIKIWFQNRRMKWKK

SEQ ID NO: 112 - protein, 27 aa, Artificial Sequence
  P7-D4-Pen
ITVDASVTLTGRQIKIWFQNRRMKWKK

SEQ ID NO: 113 - protein, 27 aa, Artificial Sequence
  P7-S4-Pen
ITVSASVTLTGRQIKIWFQNRRMKWKK

SEQ ID NO: 114 - protein, 27 aa, Artificial Sequence
  P7-G10-Pen
ITVYASVTLGGRQIKIWFQNRRMKWKK

SEQ ID NO: 115 - protein, 27 aa, Artificial Sequence
  P7-N10-Pen
ITVYASVTLNGRQIKIWFQNRRMKWKK

SEQ ID NO: 116 - protein, 27 aa, Artificial Sequence
  P7-D10-Pen
ITVYASVTLDGRQIKIWFQNRRMKWKK

SEQ ID NO: 117 - protein, 27 aa, Artificial Sequence
  P7-R10-Pen
ITVYASVTLRGRQIKIWFQNRRMKWKK

SEQ ID NO: 118 - protein, 10 aa, Artificial Sequence
  P7-K2
IKVYASVTLT

SEQ ID NO: 119 - protein, 10 aa, Artificial Sequence
  P7-N4
ITVNASVTLT

SEQ ID NO: 120 - protein, 10 aa, Artificial Sequence
  P7-D4
ITVDASVTLT

SEQ ID NO: 121 - protein, 10 aa, Artificial Sequence
  P7-S4
ITVSASVTLT